(19) United States
(12) Patent Application Publication
(10) Pub. No.: US 2025/0340864 A1
(43) Pub. Date: Nov. 6, 2025

(54) SINGLE-NUCLEUS HIGH-RESOLUTION MULTI-MODAL SPATIAL GENOMICS

(71) Applicants: THE BROAD INSTITUTE, INC., Cambridge, MA (US); PRESIDENT AND FELLOWS OF HARVARD COLLEGE, Cambridge, MA (US); THE GENERAL HOSPITAL CORPORATION, Boston, MA (US)

(72) Inventors: Fei CHEN, Cambridge, MA (US); Evan MACOSKO, Boston, MA (US); Andrew RUSSELL, Cambridge, MA (US); Naeem NADAF, Cambridge, MA (US); Jackson WEIR; Vipin KUMAR, Cambridge, MA (US)

(73) Assignees: THE BROAD INSTITUTE, INC., Cambridge, MA (US); PRESIDENT AND FELLOWS OF HARVARD COLLEGE, Cambridge, MA (US); THE GENERAL HOSPITAL CORPORATION, Boston, MA (US)

(21) Appl. No.: 19/244,580

(22) Filed: Jun. 20, 2025

Related U.S. Application Data
(63) Continuation of application No. PCT/US2023/085534, filed on Dec. 21, 2023.
(60) Provisional application No. 63/434,345, filed on Dec. 21, 2022.

(51) Int. Cl. C12N 15/10

(52) U.S. Cl. CPC C12N 15/1065; C12N 15/1068

(57) ABSTRACT

Embodiments disclosed herein provide for spatially tagged nuclei that are compatible with any genomic or multiomic single cell/nuclei assay to allow generation of a spatially resolved single cell sequencing library with single cell resolution.

### CROSS-REFERENCE TO RELATED APPLICATIONS

This application is a continuation under 35 U.S.C. § 111 (a) of International Application No. PCT/US2023/085534, filed Dec. 21, 2023, which claims the benefit of U.S. Provisional Application No. 63/434,345, filed Dec. 21, 2022. The entire contents of the above-identified applications are hereby fully incorporated herein by reference.

### STATEMENT REGARDING FEDERALLY SPONSORED RESEARCH

This invention was made with government support under Grant Nos. HG010647, CA246632, and NS132135 awarded by the National Institutes of Health. The government has certain rights in the invention.

### REFERENCE TO AN ELECTRONIC SEQUENCE LISTING

The contents of the electronic sequence listing (BROD-5740WP_ST26.xml; size 26,852, and it was created on Dec. 21, 2023), is herein incorporated by reference in its entirety.

### TECHNICAL FIELD

The subject matter disclosed herein is generally directed to spatially resolved single nuclei sequencing libraries obtained using compositions and methods for tagging nuclei with spatial barcodes.

### BACKGROUND

Recent technological innovations have enabled the high-throughput quantification of gene expression and epigenetic regulation within individual cells, transforming the understanding of how complex tissues are constructed. Missing from these measurements, however, is the ability to routinely and easily spatially localize profiled cells. Approaches for spatial monitoring of genomics data in single cells in a tissue sample include traditional histological approaches, in which sections of tissue are fixed, stained, and assessed, e.g., for the presence of individual transcripts across the viewable region of the fixed tissue section on a microscope slide, as well as certain more recent in situ techniques for transcriptome monitoring, which have thus far been afflicted by being laborious in application, offering a low degree of multiplexing with a high degree of technical difficulty and/or providing only low resolution of spatial capture across an array (i.e., providing only approximately 100-200 μm resolution).

There is a need for spatially-resolved capture of macromolecules from single-nuclei. Current spatially-resolved (multi) omics tools capture an undefined proportion of each cell as they use thin tissue section inputs, where incomplete cells/nuclei that have been cut by this procedure are captured (e.g., in situ sequencing, in situ capturing). Furthermore, some technologies profile macromolecules in voxels that can comprise multiple cells and require deconvolution (e.g., 10×Visium, Slide-seq, HOST, LCM), limiting some analyses. Recent technologies (e.g., sci-Space, XYZeq) allow capture of transcriptomic data from single-nuclei whilst retaining spatial information but are limited by low capture rate and low spatial resolution. Furthermore, they are limited to single-combinatorial-indexing based profiling.

A need therefore exists for improved approaches that provide spatial genomics profiling at resolutions approaching single cell resolution. More generally, a need also exists for improved approaches that provide spatial macromolecule abundance data (e.g., RNA expression, DNA and/or protein abundance) at resolutions approaching single cell resolution.

Citation or identification of any document in this application is not an admission that such a document is available as prior art to the present disclosure.

### SUMMARY

In one aspect, the present disclosure provides for a method of generating spatially tagged nuclei for use in single cell genomics comprising: placing a tissue sample on a sequence verified spatial array, wherein the spatial array comprises nucleic acid sequences comprising spatial barcodes coupled to the array via cleavable linkers, wherein the spatial barcodes are the same for an individual location on the array, but are different for any other location on the spatial array; cleaving the linkers and delivering the spatial barcode nucleic acids to nuclei in the tissue sample; and isolating tagged nuclei from the tissue sample. In certain embodiments, the method further comprises preparing a single cell genomics sequencing library using the isolated tagged nuclei, wherein nucleic acid sequences, each comprising a cell of origin identifying cell barcode sequence, and, optionally, a unique molecular identifier (UMI), capture the spatial barcode sequence from each spatially tagged nuclei to create a nucleic acid sequence comprising a spatial barcode and cell barcode, such that genomics data for each single cell can be identified by the cell barcode and the spatial location of the same single cell in the tissue can be identified by the same cell barcode.

In certain embodiments, the spatial barcodes are delivered to the nuclei by diffusion. In certain embodiments, before step (a) the spatial array is sequence verified by in situ sequencing of the nucleic acid sequences comprising spatial barcodes, whereby an index of the spatial barcodes on the array is generated. In certain embodiments, in situ sequencing is performed by sequencing by ligation or sequencing by synthesis.

In certain embodiments, the spatial array comprises solid supports fixed at each location on the array and coupled via the cleavable linkers to the nucleic acid sequences comprising spatial barcodes, wherein the spatial barcodes are the same for an individual solid support, but are different for any other solid support in the spatial array. In certain embodiments, the solid supports are covalently fixed to the spatial array. In certain embodiments, the solid supports are fixed to the spatial array with a vinyl polymer. In certain embodiments, the solid supports are beads. In certain embodiments, the beads are polystyrene beads. In certain embodiments, the beads are 50 μm or less in diameter. In certain embodiments, the beads are 1, 3, 10, 15, or 20 μm in diameter.

In certain embodiments, the linkers are photocleavable, chemically cleavable, or enzymatically cleavable linkers. In certain embodiments, the spatial barcode nucleic acids comprise poly-A sequences for capture by a cell barcode nucleic acid comprising a poly-T sequence. In certain embodiments, the tissue sample is treated to permeabilize the nuclei. In certain embodiments, the tissue sample is permeabilized in a buffer that increases diffusion of the spatial barcode nucleic acids. In certain embodiments, the lengths of the spatial barcode nucleic acids are different. In certain embodiments, the spatial barcode nucleic acids further comprise one or more modifications that enhance diffusion to nuclei. In certain embodiments, the spatial barcode nucleic acids are modified by addition of one or more lipid or cholesterol groups. In certain embodiments, the spatial barcode nucleic acids further comprise one or more fluorescent labels that can be used to identify tagged nuclei.

In certain embodiments, the tissue sample is a fresh frozen tissue section. In certain embodiments, the tissue sample is a fresh unfixed tissue section. In certain embodiments, the tissue sample is a fixed tissue section.

In certain embodiments, the location of each cell in the tissue sample is computationally determined based on sequencing of the library. In certain embodiments, the cell barcode nucleic acids comprise UMI sequences and the location of each cell in the tissue is determined based on the number of UMIs sequenced for each spatial barcode having the same cell barcode sequence.

In certain embodiments, the single cell genomics sequencing library is a single nucleus RNA-sequencing library (snRNA-seq). In certain embodiments, the single cell genomics sequencing library is a single cell DNA accessibility library. In certain embodiments, the single cell genomics sequencing library is a single cell ATAC-sequencing library (ATAC-seq). In certain embodiments, the single cell genomics sequencing library is a single cell chromatin immunoprecipitation (ChIP) sequencing library. In certain embodiments, the single cell genomics sequencing library is a single cell genome sequencing library. In certain embodiments, the single cell genomics sequencing library is a single cell DNA-methylation sequencing library. In certain embodiments, the single cell genomics sequencing library is a single cell Hi-C sequencing library. In certain embodiments, the single cell genomics sequencing library is a single cell enzyme-tethering chromatin profiling sequencing library. In certain embodiments, the single cell genomics sequencing library is a single cell genome and transcriptome sequencing library (G&T-seq). In certain embodiments, the single cell genomics sequencing library is a single cell proteomic library.

In another aspect, the present disclosure provides for a kit comprising a plurality of solid supports coupled via cleavable linkers to nucleic acid sequences comprising spatial barcodes and a capture sequence, wherein the spatial barcodes are the same for an individual solid support, but are different for any other solid support in the spatial array, and wherein the capture sequence is the same across all of the solid supports. In certain embodiments, the solid supports further comprise a chemical binding moiety for covalently fixing the solid supports to an array. In certain embodiments, the solid supports are beads. In certain embodiments, the beads are polystyrene beads. In certain embodiments, the beads are 50 μm or less in diameter. In certain embodiments, the beads are 1, 3, 10, 15, or 20 μm, preferably, 10 μm in diameter. In certain embodiments, the linkers are photocleavable, chemically cleavable, or enzymatically cleavable linkers. In certain embodiments, the capture sequence comprises a poly-A sequence. In certain embodiments, the lengths of the spatial barcode nucleic acids coupled to the solid supports are different. In certain embodiments, the spatial barcode nucleic acids further comprise one or more modifications that enhance diffusion to nuclei. In certain embodiments, the spatial barcode nucleic acids are modified by addition of one or more lipid or cholesterol groups. In certain embodiments, the spatial barcode nucleic acids further comprise one or more fluorescent labels that can be used to identify tagged nuclei.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1 is a diagram showing a Slide-tags protocol.

FIG. 2A-2C show Slide-tag molecular reactions. FIG. 2A shows the steps of (SEQ ID NO: 1-7) an oligonucleotide cleaved from beads are used to tag nuclei, tagged nuclei are encapsulated in droplets with cell barcode beads, and mRNA and spatial barcodes are captured by cell barcode beads. FIG. 2B shows the step of (SEQ ID NO: 6-12) separating spatial barcodes and RNA by size. FIG. 2C shows (SEQ ID NO: 13-21) the generation of sequencing libraries.

FIG. 3 shows a computational workflow to generate a spatially resolved single nuclei RNA-seq map.

FIG. 4 shows a comparison of Slide-tags to other spatial technologies.

FIG. 5A-5C show Slide-tags results in the mouse hippocampus. FIG. 5A shows UMAP plots showing cells identified from snRNA-seq. FIG. 5B shows expression of marker genes projected on UMAP plots. FIG. 5C shows mapping of the cell types on the tissue sample.

FIG. 6 shows volcano plots showing increased signal and percentage of cells that can be spatially localized since first slide-tag experiment.

FIG. 7A-7B show Slide-tags results in the mouse hippocampus. FIG. 7A shows a Slide-tag spatial map compared to immunohistochemistry of sections from the same specimen using the same marker genes. FIG. 7B shows spatial location of specific cell types.

FIG. 8A-8G show Slide-tags results in mouse embryos. FIG. 8A shows an image of a section of an embryo and a UMAP plot showing single nuclei clustering based on snRNA-seq data from the section. FIG. 8A also shows a UMAP plot showing the cell type and a graph showing percentage of cell types. FIG. 8B shows spatial localization of the nuclei on the section. FIG. 8C shows spatial localization of brain regions identified with slide-tags as compared to a histology image. FIG. 8D shows an image of slide-tags UMIs in a section and histology images of each section location. FIG. 8E shows images of slide-tags spatial location of nuclei that express the indicated marker gene in a section and a histology image of the section location. FIG. 8F shows spatial location of cell types in the section. Spatial location of clusters of different neurons are also shown. FIG. 8G shows spatial location of individual neuron clusters.

FIG. 9A-9B show Slide-tags pseudotime results. FIG. 9A shows panels showing dynamic events during neuron development. FIG. 9B shows UMAP plots and specific clusters analyzed by pseudotime, spatially resolved pseudotime analysis, and expression of specific markers during pseudotime.

FIG. 10 shows Slide-tags pseudotime results. FIG. 10 shows a UMAP plot showing pseudotime values and spatial location of pseudotime values.

FIG. 11 shows Slide-tags results in mouse embryos. Spatial location of cell types in a lateral section and spatial location of clusters of different neurons are also shown.

FIG. 12 shows a panel describing 3 dimensional (3D) slide-tags.

FIG. 13A-13C shows a comparison of tissue section thickness. FIG. 13A shows illustrations showing nuclei in tissue sections. FIG. 13B shows a UMAP plot of nuclei from a 20 μm section and a graph showing percentage of each cell type. FIG. 13C shows a UMAP plot of nuclei from a 40 μm section and a graph showing percentage of each cell type.

FIG. 14A-14B show a plate-based slide-tag. FIG. 14A (SEQ ID NO: 22) shows a flow diagram showing single cell genome and transcriptome analysis. FIG. 14B shows a flow diagram showing use of tagged nuclei to determine spatially resolved genome and transcriptome data.

FIG. 15A shows capture of spatial barcodes and mRNA in droplets (10×GEMS and beads). The 10×beads include capture sequence 1 oligonucleotides for capturing spatial barcodes and poly dT oligonucleotides for capturing mRNA. Template switching oligonucleotides (TSO) add an adapter sequence to the RT product and a spatial barcode sequencing library and gene expression library is obtained. FIG. 15B shows reverse transcription and extension performed in the GEM droplet. FIG. 15C shows cDNA amplification of the RT products. FIG. 15D shows sample index libraries prepared.

FIG. 16A shows a schematic of a slide-tags experiment to profile the mouse hippocampus. Output from a snRNA-seq experiment is a cells x gene matrix and an accompanying spatial coordinate matrix. FIG. 16B shows a dimensionality reduction plot highlighting cell type populations and each of these cells has an associated cluster of spatial barcodes.

FIG. 17 shows Slide-tags enables single-nucleus spatial transcriptomics in the mouse hippocampus. FIG. 17 shows a Slide-tag experiment showing localization of nuclei to spatial coordinates in the mouse hippocampus; cells are shaded according to cell type annotation. Spatial expression of known marker genes compared with in situ hybridization data from the Allen Mouse Brain Atlas (scales, normalized average counts).

FIG. 18 shows spatial resolution measurements in the mouse hippocampus and Slide-tags snRNA-seq enables characterization of the deep and superficial sublayers in the mouse hippocampal CA1 field. A 10 um nissl-stained section (left) was taken adjacently to the Slide-tags profiled section (right). The CA1 nuclei were subsetted in each case and a line was fitted to measure the midpoint of this structure. For Slide-tags, nuclei were selected based on their cell type assignment, with 2 spatial outliers removed. For Nissl, nuclei were computationally segmented. Orthogonal distances from this midpoint were then calculated and points are shaded by this distance.

FIG. 19 shows spatial resolution measurements in the mouse hippocampus and Slide-tags snRNA-seq enables characterization of the deep and superficial sublayers in the mouse hippocampal CA1 field. FIG. 19 shows a PCA plot showing cells from the CA1 cluster after subsetting, reprojection, and reclustering. Cells are shaded according to their new sub-cluster assignment. Cells are plotted according to their spatial location. Violin plots showing gene expression differences between each subcluster and the expression of these genes spatially.

FIG. 20 shows Slide-tags provides high molecular quality and spatial resolution. FIG. 20 is a schematic showing that each nucleus receives many spatial barcode oligos from many different beads. FIG. 20 includes a graph showing an estimate of the spatial resolution less than 10 microns.

FIG. 21 shows comparison metrics plotted for snRNA-seq compared with Slide-tags snRNA-seq, performed on consecutive sections. Cell type proportions and mean UMIs per cell are plotted by cell type. The normalized average UMI counts were determined per gene across all cells. Normalized average counts were compared.

FIG. 22 shows Slide-tags generates high quality spatial single cell data. Violin plots of log₁₀-transformed genes and UMIs per nucleus (Slide-tags) or 20 μm spatial spot (Slide-seqV2, DBiT-seq, or Xenium) in the mouse brain are shown. FIG. 22 also includes an elbow plot of standard deviations of principal components from Slide-tags snRNA-seq, Slide-seqV2, DBiT-seq, and Xenium in the mouse brain.

FIG. 23 shows Slide-tags expands the spatial genomics repertoire at single-cell resolution. FIG. 23 is a schematic showing that slide-tagged nuclei can be used for different single cell technologies.

FIG. 24A-24B show spatially-resolved snRNA-seq in a genetically heterogeneous sample. Slide tags was performed on a human metastatic melanoma sample. FIG. 24A is a schematic showing experimental design and an H&E image of melanoma sample. FIG. 24B shows transcriptomic clusters and spatial location of single cells.

FIG. 25A-25B shows spatial segregation of distinct tumour subpopulations. FIG. 25A shows mapping of transcriptionally distinct melanoma subpopulations in space. FIG. 25B shows copy number variation was inferred from the transcriptome data using inferCNV.

FIG. 26A-26B shows tumor clusters have distinct spatial neighborhoods. FIG. 26A shows a plot showing the proportion of neighbors that the tumor cells have. The y-axis shows tumor clusters and the x-axis shows immune cell types. FIG. 26B shows a spatial plot of the enrichment in the fraction of CD8_T cells within each tumors neighborhood.

FIG. 27A-27B show differential immune states and receptors between tumor clones. FIG. 27A shows the differential expression in T cells between tumor lobes. FIG. 27B (SEQ ID NO: 23-24) shows T cell receptors (TCRs) recovered from slide-tags for tumor 1 and tumor 2. FIG. 27B also shows the TCR clones spatially mapped on tumor 1 and tumor 2.

FIG. 28 shows HLA locus downregulation in tumour 1. Copy number variation was inferred from the transcriptome data using inferCNV (Chr6 is boxed).

FIG. 29A-29C show spatial multiomic sequencing with Slide-tags. FIG. 29A shows clustering of single cells by RNA expression and chromatin accessibility using slide-tags. FIG. 29B shows chromatin accessibility in single cell types and single gene expression by violin plots for each cell type. FIG. 29C shows a spatial map of the single cells.

FIG. 30A-30C show Epigenomic differences between tumor 1 and tumor 2 populations. FIG. 30A shows a plot of a comparison of ATAC-seq and RNA expression in single cells (TNC is bolded). FIG. 30B shows chromatin accessibility in each tumor and gene expression for TNC shown by violin plots for each tumor. FIG. 30C shows a spatial map of single cells by TNC chromatin gene score in tumor 1 and tumor 2.

FIG. 31A-31B shows Tumour 1 cell state driven by spatially clustered TF motifs. FIG. 31A shows expression of mesenchymal-like cell state genes in tumor 1 and tumor 2. FIG. 31B is a plot showing transcription factor motifs identified by ATAC-seq positively and negatively correlated with the mesenchymal score.

FIG. 32A-32G show Slide-tags profiling of postmortem human cortex. FIG. 32A is slide-tags performed on a 5.5×5.5 area of the human prefrontal cortex. FIG. 32B is a spatial map showing all cortical cell types. FIG. 32C is a spatial map showing gene markers for cortical cell types. FIG. 32D shows a spatial map and plot showing all cortical layered types. FIG. 32E is a spatial map showing CUx2 expression. FIG. 32F is a spatial map showing RORB expression. FIG. 32G is a spatial map showing FOXP2 expression.

FIG. 33A-33B show Slide-tags profiling of human tonsil. FIG. 33A shows snRNA-seq data from the tonsil. Clustering by cell type is shown. FIG. 33B is a spatial map of the single cells and H and E staining of tissue.

FIG. 34A-34C show using spatially varying genes to identify dark zone and light zones in germinal centers. FIG. 34A is a schematic of a germinal center having a light and dark zone. FIG. 34B shows Slide-tags of a germinal center was used to cluster cell types. FIG. 34C shows B cell clusters and expression of marker genes and spatial maps of germinal center B cells.

FIG. 35A-35B shows nominating spatially significant receptor ligand interactions. FIG. 35A shows prior methods of inferring receptor ligand interactions using single cell data. FIG. 35B shows using spatial information to curate receptor ligand interactions in germinal centers.

FIG. 36A-36B shows Slide-tags yields high quality spatial multiomic data. FIG. 36A is a schematic showing experimental design for multimodal analysis of the P1 mouse brain. FIG. 36B shows single cell ATAC-seq and snRNA-seq from the same single nuclei.

FIG. 37A-37B shows Slide-tags allows spatial ATAC & RNA-seq in the same single cells. FIG. 37A shows clustering of single nuclei by RNA expression, accessible chromatin (ATAC), and weighted-nearest neighbor (WNN) analysis (see, e.g., Hao Y, Hao S, Andersen-Nissen E, et al. Integrated analysis of multimodal single-cell data. Cell. 2021; 184(13):3573-3587.e29). FIG. 37B shows plots of the clusters in (A) in space.

FIG. 38A-38D show transcription and open chromatin can define spatially distinct elements. FIG. 38A shows a spatial map of the outer layer of the developing cortex (the isocortex, the retrosplenal cortex, and the subiculum) using RNA expression. FIG. 38B shows representative top RNA hits. FIG. 38C shows clustering using ATAC and top chromatin accessibility hits. FIG. 38D shows transcription factor motifs that vary along this axis and top hits.

FIG. 39 shows oligonucleotide variations for Slide-tags. (SEQ ID NO: 2-3, 25-28) Capture sequence 1 can be the capture sequence on the spatial barcode oligonucleotides. Capture sequence 1 is complementary to the 10×Genomics chromium 3′ v3.1 platform. Oligonucleotide variation 1 shows adding a capture sequence using a 10×capture sequence 1 and a splint. Oligonucleotide variation 2 shows a spatial barcode oligonucleotide having capture sequence 1 downstream of the spatial barcode. Oligonucleotide variation 3 shows the capture sequence as a polyA sequence used to capture a poly T sequence. Oligonucleotide variation 4 shows a chemical modification that can be present on any of the spatial barcode oligonucleotides to facilitate nuclear uptake or retention (e.g., Cholesterol-TEG, Squalene, Fatty Acids, or alpha-Tocopherol). Oligonucleotide variation 5 shows a chemical modification that can be present on any of the spatial barcode oligonucleotides to facilitate detection and quantification of spatial barcodes (e.g., fluorescent dye, tri-functional linker which can have a combination of fluorescent dye and one of the chemical modifications listed above.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2^{nd} ed., J. Wiley & Sons (New York, N.Y. 1994), March, Advanced Organic Chemistry Reactions, Mechanisms and Structure 4^{th} ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosure. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present disclosure encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, and cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

Reference is made to US Patent Application publication number US20210123040A1. Reference is also made to “Slide-tags: scalable, single-nucleus barcoding for multi-modal spatial genomics,” Andrew J. C. Russell, Jackson A. Weir, Naeem M. Nadaf, Matthew Shabet, Vipin Kumar, Sandeep Kambhampati, Ruth Raichur, Giovanni J. Marrero, Sophia Liu, Karol S. Balderrama, Charles R. Vanderburg, Vignesh Shanmugam, Luyi Tian, Catherine J. Wu, Charles H. Yoon, Evan Z. Macosko, Fei Chen, bioRxiv 2023.04.01.535228. Reference is also made to Russell, A. J. C., Weir, J. A., Nadaf, N. M. et al. Slide-tags enables single-nucleus barcoding for multimodal spatial genomics. Nature (2023). All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide for spatially tagged nuclei that are compatible with any genomic or multiomic single cell/nuclei assay to allow generation of a spatially resolved single cell sequencing library with single cell resolution. Previous methods were limited to detecting mRNA expression and were also limited by the amount of mRNA diffused from the cell and captured (see, e.g., Stickels R R, Murray E, Kumar P, et al. Highly sensitive spatial transcriptomics at near-cellular resolution with Slide-seqV2. Nat Biotechnol. 2021; 39(3):313-319). The present disclosure overcomes these limitations by tagging nuclei with spatial barcodes, such that capturing analytes is not dependent upon diffusion from a cell. The nuclei can be completely lysed in a reaction volume to release all analytes (e.g., RNA), such that all RNA can be captured. Further, in order to perform multiomic spatial studies using slide-seq, each omic measurement has to be performed separately. The present disclosure does not require performing every spatial experiment twice or multiple times to obtain multiomic spatial results. The present disclosure is based on the use of modified slide-seq arrays (polystyrene support beads with barcoded oligonucleotides) to deliver spatial barcodes to nuclei within fresh-frozen thin tissue sections. The use of slide-seq arrays and the lack of tissue fixation distinguishes this disclosure from XYZeq and sci-Space. In addition, the structure of the spatial barcodes allows either: plate-based, microfluidic-based, or nanowell-based capture of macromolecules and spatial barcodes from single-nuclei, in contrast to XYZeq and sci-Space which require split-pool index based profiling.

The current method requires only that one or more spatial barcode tags are delivered to permeabilized nuclei. The tagged nuclei can then be stored or directly used in any single cell genomics assay. In other words, the present disclosure unifies spatial profiling and single cell sequencing methods. Further, the present disclosure provides for an array such that the distance between different spatial barcodes is less than the size of a cell allowing single cell resolution. Further, the use of fluorescently labeled spatial barcode tags allows for only tagged nuclei to be used in subsequent single cell genomics assays. Thus, because single tagged nuclei are used as the input for single nuclei/cell genomic assays, the analyte capture efficiency approaches the detection efficiency of the non-spatially resolved single-cell genomic sequencing techniques. For example, the RNA capture rate approaches ˜100% of single nuclei RNA sequencing data.

Tagging Nuclei in a Tissue Sample with Spatial Barcodes

In example embodiments, a tissue sample is placed on an array comprising spatial barcodes and the spatial barcodes are released from the array to tag the nuclei in the tissue. The tissue sample is permeabilized to allow the spatial barcodes to tag the nuclei. Tagged single nuclei are then isolated from the tissue sample.

### Spatial Arrays

In example embodiments, nuclei in a tissue sample are tagged with spatial barcode nucleic acids. The spatial barcode nucleic acids are nucleic acids linked or attached to an array at specific positions and that include barcode sequences. Thus, the spatial barcode can identify the position in the array. As used herein, the term “array” refers to a population of features or sites that can be differentiated from each other according to relative location. Different molecules that are at different sites of an array can be differentiated from each other according to the locations of the sites in the array. An individual site of an array can include one or more molecules of a particular type. For example, a site can include a single target nucleic acid molecule having a particular sequence or a site can include several nucleic acid molecules having the same sequence (and/or complementary sequence, thereof). The sites of an array can be different features located on the same substrate. Exemplary features include without limitation, wells in a substrate, beads (or other particles) in or on a substrate, projections from a substrate, ridges on a substrate or channels in a substrate. The sites of an array can be separate substrates each bearing a different molecule. Different molecules attached to separate substrates can be identified according to the locations of the substrates on a surface to which the substrates are associated or according to the locations of the substrates in a liquid or gel. Exemplary arrays in which separate substrates are located on a surface include, without limitation, those having beads in wells, beads arranged upon a flat surface (e.g., a slide), optionally beads captured upon a flat surface (e.g., a layer of beads adhered to or otherwise stably associated with a slide (e.g., a layer of beads adsorbed to a slide-attached elastomeric surface)), etc. In example embodiments, the array of the present disclosure includes greater than 10,000 individual locations, each location having a different spatial barcode. In example embodiments, the array of the present disclosure includes 10,000 to more than 1 million individual locations, each location having a different spatial barcode.

As used herein, the term “feature” means a location in an array for a particular species of molecule. A feature can contain only a single molecule, or it can contain a population of several molecules of the same species. Features of an array are typically discrete. The discrete features can be contiguous, or they can have spaces between each other. The size of the features and/or spacing between the features can vary such that arrays can be high density, medium density or lower density. High density arrays are characterized as having sites separated by less than about 15 μm. Medium density arrays have sites separated by about 15 to 30 μm, while low density arrays have sites separated by greater than 30 μm. An array useful herein can have, for example, sites that are separated by less than 100 μm, 50 μm, 10 μm, 5 μm, 1 μm, or 0.5 μm. An apparatus or method of the present disclosure can be used to detect an array at a resolution sufficient to distinguish sites at the above densities or density ranges.

As used herein, the term “attached” refers to the state of two things being joined, fastened, adhered, connected or bound to each other. For example, an analyte, such as a nucleic acid, can be attached to a material, such as a gel or solid support, by a covalent or non-covalent bond. A covalent bond is characterized by the sharing of pairs of electrons between atoms. A non-covalent bond is a chemical bond that does not involve the sharing of pairs of electrons and can include, for example, hydrogen bonds, ionic bonds, van der Waals forces, hydrophilic interactions and hydrophobic interactions.

### Spatial Barcode Nucleic Acids

In example embodiments, a spatial barcode nucleic acid is a nucleic acid sequence that includes a barcode sequence. In example embodiments, a spatial barcode nucleic acid is a nucleic acid sequence that includes from 5′ to 3′: a cleavable linker, a barcode sequence, and a capture sequence. In example embodiments, the spatial barcode nucleic acids are 3′ end blocked to prevent extension. Thus, upon capture by a cell barcode, the cell barcode sequence primes extension into the spatial barcode nucleic acid to add the spatial barcode sequence to the cell barcode sequence and the spatial barcode nucleic acid is not extended. In example embodiments, the spatial barcode nucleic acids include a barcode sequence that identifies the location on the array. The term “barcode” as used herein refers to a short sequence of nucleotides (for example, DNA or RNA) or a series of nucleotides in a nucleic acid that can be used to identify the nucleic acid, a characteristic of the nucleic acid (e.g., the identity and optionally the location of the nucleic acid), or a manipulation that has been carried out on the nucleic acid (e.g., a perturbation) that is used as an identifier for an associated molecule, such as a target molecule and/or target nucleic acid, or as an identifier of the source of an associated molecule, such as a cell-of-origin. As used herein, the term “spatial barcode” or “spatial tag” is intended to mean a series of nucleotides in a nucleic acid that can be used to identify the location on an array to which a nucleic acid is fixed. As used herein, the term “spatial barcode” or “spatial tag” is also intended to mean a nucleic acid having a sequence that is indicative of a location. Typically, the nucleic acid is a synthetic molecule having a sequence that is not found in one or more biological specimen that will be used with the nucleic acid. However, in some embodiments the nucleic acid molecule can be naturally derived, or the sequence of the nucleic acid can be naturally occurring, for example, in a biological specimen that is used with the nucleic acid. The location indicated by a spatial tag can be a location in or on a biological specimen, in or on a solid support or a combination thereof. A barcode sequence can function as a spatial tag. In example embodiments, the identification of the location of the tag that serves as a spatial tag is only determined after a population of beads (each possessing a distinct barcode sequence) has been arrayed upon a solid support (optionally randomly arrayed upon a solid support) and sequencing of such a bead-associated barcode sequence has been determined in situ upon the solid support.

The barcode sequence can be a naturally occurring sequence or a sequence that does not occur naturally in the organism from which the barcoded nucleic acid was obtained or from which the sample that is tagged was obtained. A barcode sequence can be unique to a single nucleic acid species in a population or a barcode sequence can be shared by several different nucleic acid species in a population (e.g., all nucleic acid species attached to an array at a defined location or a single bead might possess the same barcode sequence, while different defined locations or beads present a different shared barcode sequence that serves to identify each such different location or bead). By way of further example, each nucleic acid probe in a population can include different barcode sequences from all other nucleic acid probes in the population. Alternatively, each nucleic acid in a population can include different barcode sequences from some or most other nucleic acids in a population. In particular embodiments, one or more barcode sequences that are used with a biological specimen (e.g., a tissue sample) are not present in the genome, transcriptome or other nucleic acids of the biological specimen. For example, barcode sequences can have less than 80%, 70%, 60%, 50% or 40% sequence identity to the nucleic acid sequences in a particular biological specimen. As used herein, the term “different”, when used in reference to nucleic acids, means that the nucleic acids have nucleotide sequences that are not the same as each other. Two or more nucleic acids can have nucleotide sequences that are different along their entire length. Alternatively, two or more nucleic acids can have nucleotide sequences that are different along a substantial portion of their length. For example, two or more nucleic acids can have target nucleotide sequence portions that are different for the two or more molecules while also having a universal sequence portion that is the same on the two or more molecules.

As used herein, the term “biological specimen” is intended to mean one or more cell, tissue, organism or portion thereof. A biological specimen can be obtained from any of a variety of organisms. Exemplary organisms include, but are not limited to, a mammal such as a rodent, mouse, rat, rabbit, guinea pig, ungulate, horse, sheep, pig, goat, cow, cat, dog, primate (i.e., human or non-human primate); a plant such as Arabidopsis thaliana, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish; a reptile; an amphibian such as a frog or Xenopus laevis; a Dictyostelium discoideum; a fungi such as Pneumocystis carinii, Takifugu rubripes, yeast, Saccharamoyces cerevisiae or Schizosaccharomyces pombe; or a Plasmodium falciparum. Target nucleic acids can also be derived from a prokaryote such as a bacterium, Escherichia coli, Staphylococci or Mycoplasma pneumoniae; an archae; a virus such as Hepatitis C virus or human immunodeficiency virus; or a viroid. Specimens can be derived from a homogeneous culture or population of the above organisms or alternatively from a collection of several different organisms, for example, in a community or ecosystem.

In example embodiments, the spatial barcode nucleic acid includes a universal sequence used to capture the spatial barcode nucleic acid onto another nucleic acid sequence (e.g., a cell of origin identifying barcode nucleic acid). In some embodiments, the universal sequence is also referred to as a capture sequence, or handle sequence, such as a ligation, PCR, or indexing handle sequence. In preferred embodiments, the capture sequence is at the 3′ end of the spatial barcode nucleic acids. In example embodiments, the spatial barcode nucleic acid also includes a universal sequence used as a primer binding sequence. As used herein, the term “universal sequence” refers to a series of nucleotides that is common to two or more nucleic acid molecules even if the molecules also have regions of sequence that differ from each other. A universal sequence that is present in different members of a collection of molecules can allow capture of multiple different nucleic acids using a population of universal capture nucleic acids that are complementary to the universal sequence. Similarly, a universal sequence present in different members of a collection of molecules can allow the replication or amplification of multiple different nucleic acids using a population of universal primers that are complementary to the universal sequence. Thus, a universal capture nucleic acid or a universal primer includes a sequence that can hybridize specifically to a universal sequence. Target nucleic acid molecules may be modified to attach universal adapters, for example, at one or both ends of the different target sequences. Non-limiting examples of 3′ universal sequences used for capture of the spatial barcode nucleic acids include poly-A sequences for capture by poly-T sequences, or sequences complementary to commercially available capture sequences, such as tagmentation adapter sequences (e.g., Read 1 (Read IN) sequence on the beads in the Chromium Next GEM Single Cell ATAC Reagent Kit v1.1 (10× Genomics, Pleasanton, CA, USA)).

As used herein, the term “poly-T or poly-A,” when used in reference to a nucleic acid sequence, is intended to mean a series of two or more thiamine (T) or adenine (A) bases, respectively. A poly-T or poly-A can include at least about 2, 5, 8, 10, 12, 15, 18, 20, 25, 30 or more of the T or A bases, respectively. Alternatively or additionally, a poly-T or poly-A can include at most about 30, 25, 20, 18, 15, 12, 10, 8, 5 or 2 of the T or A bases, respectively.

In example embodiments, the spatial barcode nucleic acids do not require a UMI sequence because a UMI sequence is present on the single cell/nuclei genomics assay capture sequence, which also includes a cell of origin barcode sequence. Thus, when the spatial barcode is captured in a single cell assay, a UMI specific to each spatial barcode capture event will be present on each sequencing read.

In example embodiments, the spatial barcode nucleic acids are about 50 to 250 nucleotides in length. In one example, the spatial barcode nucleic acids include a linker, a primer binding sequence that is the same for all spatial barcode nucleic acids, a spatial barcode that is about 6 to 50 nucleotides, preferably 6 to 20 nucleotides, and a capture sequence of about 6 to 50 nucleotides. The spatial barcode nucleic acids may also include additional sequences, for example, to change the length of the sequence. In example embodiments, the spatial barcode nucleic acids are single stranded, preferably, ssDNA. In some embodiments, single stranded oligonucleotides diffuse into nuclei better than double stranded nucleotides. In example embodiments, single-stranded DNA (ssDNA) specifically stains the nuclei of permeabilized cells but not intact cells.

In example embodiments, the spatial barcode nucleic acids on the spatial array have the same spatial barcode for each location, but have different lengths for each location. Spatial barcode nucleic acids having different lengths allow for identifying the spatial location of a single cell in 3 dimensions because shorter nucleic acids will diffuse farther into a tissue section than a longer nucleic acid. In example embodiments, computational methods can be used to determine the location of single cells in a tissue section with multiple layers of cells, such as by quantitating the number of spatial barcodes having different lengths in the single nuclei.

Barcode sequences can be any of a variety of lengths. Longer sequences can generally accommodate a larger number and variety of barcodes for a population. Generally, all probes in a plurality will have the same length barcode (albeit with different sequences), but it is also possible to use different length barcodes for different probes. A barcode sequence can be at least 2, 4, 6, 8, 10, 12, 15, 20 or more nucleotides in length. Alternatively or additionally, the length of the barcode sequence can be at most 20, 15, 12, 10, 8, 6, 4 or fewer nucleotides. Examples of barcode sequences that can be used are set forth, for example in, U.S. Patent Publication No. 2014/0342921 A1 and U.S. Pat. No. 8,460,865, each of which is incorporated herein by reference.

### Linkers

In example embodiments, the spatial barcode nucleic acids include a linker sequence for attachment to the array or a solid support (e.g., a bead described further herein). In preferred embodiments, the linker is cleavable, such that the spatial barcode nucleic acids can be released when in contact with or in proximity to a tissue specimen. In example embodiments, the cleavable linker is chemically cleavable, photocleavable, or enzymatically cleavable.

In preferred embodiments, the linker is photocleavable. Photocleavable linkers are available that can be released by UV irradiation. A PC (Photo-Cleavable) spacer can be placed between DNA bases or between the oligo and a 5′-modifier group. The spacer arm can be cleaved with exposure to UV light in the 300-350 nm spectral range. Cleavage releases the oligo with a 5′-phosphate group. An exemplary photo-cleavable linker is commercially available (Integrated DNA Technologies, Inc., Coralville, Iowa) and shown:

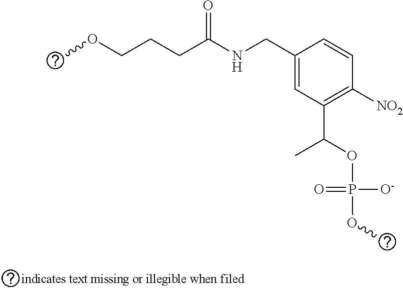

In other example embodiments, the spatial barcode nucleic acids may contain one or more cleavable linkers, e.g., that can be cleaved upon application of a suitable stimulus. For example, the cleavable sequence may be a photocleavable linker that can be cleaved by applying light, a chemical cleavable linker that can be cleaved by applying a suitable chemical, or an enzymatically cleavable linker that can be cleaved by applying an enzyme.

Oligonucleotides with photo-sensitive chemical bonds (e.g., photo-cleavable linkers) have various advantages. They can be cleaved efficiently and rapidly (e.g., in nanoseconds and milliseconds). In some cases, photo-masks can be used such that only specific regions of the array are exposed to cleavable stimuli (e.g., exposure to UV light, exposure to light, exposure to heat induced by laser). When a photo-cleavable linker is used, the cleavable reaction is triggered by light, and can be highly selective to the linker and consequently biorthogonal. Non-limiting examples of a photo-sensitive chemical bond that can be used in a cleavage domain include those described in Leriche et al. Bioorg Med Chem. 2012 Jan. 15; 20(2):571-82; U.S. Publication No. 2017/0275669; and WO2020190509A9.

### Spatial Barcode Nucleic Acid Modifications

In example embodiments, the spatial barcode nucleic acids include a nucleotide modification to enhance diffusion into nuclei. In example embodiments, the spatial barcode nucleic acids are coupled to a lipophilic or amphiphilic moiety. Lipophilic molecules can associate with and/or insert into lipid membranes such as cell membranes and nuclear membranes. Non-limiting examples of lipophilic molecules that can be used in the methods provided herein include sterol lipids such as cholesterol, tocopherol, and derivatives thereof, lignoceric acid, and palmitic acid. Other lipophilic molecules that may be used in the methods provided herein comprise amphiphilic molecules wherein the headgroup (e.g., charge, aliphatic content, and/or aromatic content) and/or fatty acid chain length (e.g., C12, C14, C16, or C18) can be varied. For instance, fatty acid side chains (e.g., C12, C14, C16, or C18) can be coupled to glycerol or glycerol derivatives (e.g., 3-t-butyldiphenylsilylglycerol), which can also comprise, e.g., a cationic head group. The spatial barcode nucleic acids disclosed herein can be coupled (either directly or indirectly) to these amphiphilic molecules. An amphiphilic molecule may associate with and/or insert into a membrane (e.g., a nuclear membrane).

A spatial barcode nucleic acid may be attached to a lipophilic moiety (e.g., a cholesterol molecule). A spatial barcode nucleic acid may be attached to the lipophilic moiety via a linker, such as a tetra-ethylene glycol (TEG) linker. Other exemplary linkers include, but are not limited to, Amino Linker C6, Amino Linker C12, Spacer C3, Spacer C6, Spacer C12, Spacer 9, Spacer 18. A spatial barcode nucleic acid may be attached to the lipophilic moiety or the linker on the 5′ end of the spatial barcode nucleic acid. The linker may be a glycol or derivative thereof. For example, the linker may be tetra-ethylene glycol (TEG) or polyethylene glycol (PEG). A spatial barcode nucleic acid may be releasably attached to the linker or lipophilic moiety (e.g., as described elsewhere herein for releasable attachment of nucleic acid molecules) such that the spatial barcode nucleic acid or a portion thereof can be released from the lipophilic molecule. In example embodiments, a lipophilic moiety (e.g., a cholesterol) is indirectly (e.g., via hybridization or ligand-ligand interactions, such as biotin-streptavidin) coupled to an oligonucleotide.

In example embodiments, the spatial barcode nucleic acids include fluorescent labels such that tagged nuclei can be identified and sorted out from non-tagged nuclei. The efficiency of nuclei tagging can also be determined by quantitating the percentage of tagged nuclei. In example embodiments, a lipophilic molecule may comprise a fluorescent moiety. Many different fluorophores can be readily attached to oligonucleotides, such as, fluorescein and its tetra- and hexachlorinated derivatives TET and HEX.

### Sequence Verified Array

In example embodiments, the spatial array is sequence verified. As used herein “sequence verified” refers to knowing the sequence of the spatial barcodes at each location in the array, or at essentially all locations, preferably greater than 50, 60, 70, 80, or 90% of locations. In an example embodiment, the array is sequence verified because the spatial barcodes attached to the array at each location were specifically placed at each location. In an example embodiment, the sequences of the spatial barcodes at each location are determined by in situ sequencing. In an example embodiment, the array is sequence verified by in situ sequencing before placing a tissue sample on the array. In example embodiments, in situ sequencing is performed by sequencing by ligation or sequencing by synthesis directly on the array and captured by microscopy.

In certain aspects of the disclosure, in situ sequencing is performed upon an array affixed to a surface, which can be performed by any art-recognized mode of parallel (optionally massively parallel) in situ sequencing, examples of which particularly include the previously described SOLID™ method, which is a sequencing-by-ligation technique that can be performed in situ upon a solid support (refer, e.g., to Voelkerding et al, Clinical Chem., 55-641-658, 2009; U.S. Pat. Nos. 5,912,148; and 6,130,073, which are incorporated herein by reference in their entireties). In certain embodiments of the instant disclosure, such sequencing can be performed upon an array present on a standard microscope slide, optionally using a standard microscope fitted with sufficient computing power to track and associate individual sequences during progressive rounds of detection, with their spatial position(s). The instant disclosure also employs fluidics, incubation times, enzymatic mixes and imaging setup in performing in situ sequencing (see, e.g., US20210123040A1).

Sequencing techniques, such as sequencing-by-synthesis (SBS) techniques, are a useful method for determining barcode sequences. SBS can be carried out as follows. To initiate a first SBS cycle, one or more labeled nucleotides, DNA polymerase, SBS primers etc., can be contacted with one or more features on a bead or other solid support (e.g. feature(s) where nucleic acid probes are attached to the bead or other solid support). Those features where SBS primer extension causes a labeled nucleotide to be incorporated can be detected. Optionally, the nucleotides can include a reversible termination moiety that terminates further primer extension once a nucleotide has been added to the SBS primer. For example, a nucleotide analog having a reversible terminator moiety can be added to a primer such that subsequent extension cannot occur until a deblocking agent is delivered to remove the moiety. Thus, for embodiments that use reversible termination, a deblocking reagent can be delivered to the bead or other solid support (before or after detection occurs). Washes can be carried out between the various delivery steps. The cycle can then be repeated n times to extend the primer by n nucleotides, thereby detecting a sequence of length n. Exemplary SBS procedures, fluidic systems and detection platforms that can be readily adapted for use with a composition, apparatus or method of the present disclosure are described, for example, in Bentley et al., Nature 456:53-59 (2008), PCT Publ. Nos. WO 91/06678, WO 04/018497 or WO 07/123744; U.S. Pat. Nos. 7,057,026, 7,329,492, 7,211,414, 7,315,019 or 7,405,281, and U.S. Patent Publication No. 2008/0108082, each of which is incorporated herein by reference.

Sequencing by ligation is a DNA sequencing method that uses the enzyme DNA ligase to identify the nucleotide present at a given position in a DNA sequence. Unlike most currently popular DNA sequencing methods, this method does not use a DNA polymerase to create a second strand. Instead, the mismatch sensitivity of a DNA ligase enzyme is used to determine the underlying sequence of the target DNA molecule. Sequencing-by-ligation reactions are also useful including, for example, those described in Shendure et al. Science 309:1728-1732 (2005); or U.S. Pat. No. 5,599,675 or 5,750,341, each of which is incorporated herein by reference. Some embodiments can include sequencing-by-hybridization procedures as described, for example, in Bains et al., Journal of Theoretical Biology 135 (3), 303-7 (1988); Drmanac et al., Nature Biotechnology 16, 54-58 (1998); Fodor et al., Science 251 (4995), 767-773 (1995); or PCT Publication No. WO 1989/10977, each of which is incorporated herein by reference. In both sequencing-by-ligation and sequencing-by-hybridization procedures, target nucleic acids (or amplicons thereof) that are present at sites of an array are subjected to repeated cycles of oligonucleotide delivery and detection. Compositions, apparatus or methods set forth herein or in references cited herein can be readily adapted for sequencing-by-ligation or sequencing-by-hybridization procedures. Typically, the oligonucleotides are fluorescently labeled and can be detected using fluorescence detectors similar to those described with regard to SBS procedures herein or in references cited herein.

A method of the present disclosure can include a step of performing a nucleic acid detection reaction on a bead or other solid support to determine barcode sequences of nucleic acid probes that are located on the bead or other solid support. In many embodiments, the probes are randomly located on the bead or other solid support and the nucleic acid detection reaction provides information to locate each of the different probes. Exemplary nucleic acid detection methods include, but are not limited to nucleic acid sequencing of a probe, hybridization of nucleic acids to a probe, ligation of nucleic acids that are hybridized to a probe, extension of nucleic acids that are hybridized to a probe, extension of a first nucleic acid that is hybridized to a probe followed by ligation of the extended nucleic acid to a second nucleic acid that is hybridized to the probe, or other methods known in the art such as those set forth in U.S. Pat. No. 8,288,103 or 8,486,625, each of which is incorporated herein by reference.

### Solid Supports

In example embodiments, the spatial barcode nucleic acids are attached via the cleavable linkers to a solid support. In one embodiment, the spatial barcode nucleic acids are attached directly to a solid support. In one embodiment, the spatial barcode nucleic acids are attached to a plurality of solid supports (e.g., beads), each of the plurality of solid supports having a unique spatial barcode sequence, which are further attached to a solid support (e.g., a slide). As used herein, the term “solid support” refers to a rigid substrate that is insoluble in aqueous liquid. The substrate can be non-porous or porous. The substrate can optionally be capable of taking up a liquid (e.g. due to porosity) but will typically be sufficiently rigid that the substrate does not swell substantially when taking up the liquid and does not contract substantially when the liquid is removed by drying. A nonporous solid support is generally impermeable to liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zeonor®, silica or silica-based materials including silicon and modified silicon, carbon, metals, inorganic glasses, optical fiber bundles, and polymers. Particularly useful solid supports for some embodiments are slides and beads capable of assorting/packing upon the surface of a slide (e.g., beads to which a large number of oligonucleotides are attached).

Any of a variety of solid supports can be used in a method, composition or apparatus of the present disclosure. Particularly useful solid supports are those used for nucleic acid arrays. Examples include glass, modified glass, functionalized glass, inorganic glasses, microspheres (e.g., inert and/or magnetic particles), plastics, polysaccharides, nylon, nitrocellulose, ceramics, resins, silica, silica-based materials, carbon, metals, an optical fiber or optical fiber bundles, polymers and multiwell (e.g., microtiter) plates. Exemplary plastics include acrylics, polystyrene, copolymers of styrene and other materials, polypropylene, polyethylene, polybutylene, polyurethanes and Teflon™ Exemplary silica-based materials include silicon and various forms of modified silicon.

In particular embodiments, a solid support can be within or part of a vessel such as a well, tube, channel, cuvette, Petri plate, bottle or the like. Optionally, the vessel is a flow-cell, for example, as described in WO 2014/142841 A1; U.S. Pat. App. Pub. No. 2010/0111768 A1 and U.S. Pat. No. 8,951,781 or Bentley et al., Nature 456:53-59 (2008), each of which is incorporated herein by reference. Exemplary flow-cells are those that are commercially available from Illumina, Inc. (San Diego, Calif.) for use with a sequencing platform such as a Genome Analyzer®, MiSeq®, NextSeq® or HiSeq® platform. Optionally, the vessel is a well in a multiwell plate or microtiter plate.

In example embodiments, a solid support can include a gel coating. Attachment, e.g., of nucleic acids to a solid support via a gel is exemplified by flow cells available commercially from Illumina Inc. (San Diego, Calif.) or described in US Pat. App. Pub. Nos. 2011/0059865 A1, 2014/0079923 A1, or 2015/0005447 A1; or PCT Publ. No. WO 2008/093098, each of which is incorporated herein by reference. Exemplary gels that can be used in the methods and apparatus set forth herein include, but are not limited to, those having a colloidal structure, such as agarose; polymer mesh structure, such as gelatin; or cross-linked polymer structure, such as polyacrylamide, SFA (see, for example, US Pat. App. Pub. No. 2011/0059865 A1, which is incorporated herein by reference) or PAZAM (see, for example, US Pat. App. Publ. Nos. 2014/0079923 A1, or 2015/0005447 A1, each of which is incorporated herein by reference).

In some embodiments, a solid support can be configured as an array of features to which beads can be attached. The features can be present in any of a variety of desired formats. For example, the features can be wells, pits, channels, ridges, raised regions, pegs, posts or the like. Exemplary features include wells that are present in substrates used for commercial sequencing platforms sold by 454 LifeSciences (a subsidiary of Roche, Basel Switzerland) or Ion Torrent (a subsidiary of Life Technologies, Carlsbad Calif.). Other substrates having wells include, for example, etched fiber optics and other substrates described in U.S. Pat. Nos. 6,266,459; 6,355,431; 6,770,441; 6,859,570; 6,210,891; 6,258,568; 6,274,320; US Pat app. Publ. Nos. 2009/0026082 A1; 2009/0127589 A1; 2010/0137143 A1; 2010/0282617 A1 or PCT Publication No. WO 00/63437, each of which is incorporated herein by reference. In some embodiments, wells of a substrate can include gel material (with or without beads) as set forth in US Pat. App. Publ. No. 2014/0243224 A1, which is incorporated herein by reference.

Features can appear on a solid support as a grid of spots or patches. The features can be located in a repeating pattern or in an irregular, non-repeating pattern. Optionally, repeating patterns can include hexagonal patterns, rectilinear patterns, grid patterns, patterns having reflective symmetry, patterns having rotational symmetry, or the like. Asymmetric patterns can also be useful. The pitch of an array can be the same between different pairs of nearest neighbor features or the pitch can vary between different pairs of nearest neighbor features.

In particular embodiments, features on a solid support can each have an area that is larger than about 100 nm², 250 nm², 500 nm², 1 μm², 2.5 μm², 5 μm², 10 μm² or 50 μm². Alternatively or additionally, features can each have an area that is smaller than about 50 μm², 25 μm², 10 μm², 5 μm², 1 μm², 500 nm², or 100 nm². The preceding ranges can describe the apparent area of a bead or other particle on a solid support when viewed or imaged from above.

In example embodiments, the present disclosure provides a method for generating and using a spatially tagged array of microbeads to perform tagging of nuclei with spatial barcode nucleic acids upon cryosectioned tissue samples, with high image resolution. The method can include the steps of (a) attaching different nucleic acid probes (spatial barcode nucleic acids) to beads that are then captured upon a solid support to produce randomly located probe-possessing beads on the solid support, wherein the different nucleic acid probes each includes a barcode sequence (that is shared by all such nucleic acid probes of a single bead), and wherein each of the randomly located beads includes a different barcode sequence(s) from other randomly located beads on the solid support; (b) performing a nucleic acid detection reaction on the solid support to determine the barcode sequences of the randomly located beads on the solid support; (c) contacting a biological specimen with the solid support that has the randomly located beads; (d) releasing the probes presented by the randomly located beads to tag the biological specimen that are proximal to the randomly located beads; and (e) isolating tagged nuclei, thereby spatially tagging the nuclei of the biological specimen.

As used herein, “beads”, “microbeads”, “microspheres” or “particles” or grammatical equivalents can include small discrete particles. The composition of the beads can vary, depending upon the class of capture probe, the method of synthesis, and other factors. In example embodiments of the instant disclosure, the sizes of the beads of the instant disclosure tend to range from 1 μm to 100 μm in diameter (with all subranges within this range expressly contemplated), e.g., depending upon the extent of image resolution desired, nature of the solid support to be used for spatial bead array construction, sequencing processes (e.g., flow cell sequencing) to be employed, as well as other factors. In example embodiments, the smaller the bead used the tighter space each spatial barcode is located and the better the resolution. Using smaller beads, in particular when the distance between different spatial barcodes is less than the size of a single cell, may result in the same nuclei being tagged with more than one spatial barcode. This case would not affect the resolution because the single cell could be located at an overlapping site on the array. In the case where the bead was larger than a single cell, the resolution would be decreased due to more than one nuclei being tagged with the same spatial barcode. In this case, a single cell could only be localized to a location on the array larger than a single cell.

Example embodiments of the instant disclosure employ a collection of beads or other particles, to which oligonucleotides are attached. The bead sizes can range from nanometers, for example, 100 nm, to millimeters, for example, 1 mm, with beads from about 0.2 μm to about 200 μm commonly employed, and from about 5 to about 20 μm being within the range currently exemplified, although in some embodiments smaller or larger beads may be used. For example, beads less than 50 μm, such as 1 μm, 3 μm, 10 μm, 15 μm, 20 μm, in particular about 10 μm.

Suitable bead compositions include those used in peptide, nucleic acid and organic moiety synthesis, including, but not limited to, plastics, ceramics, glass, polystyrene, methylstyrene, acrylic polymers, paramagnetic materials, thoriasol, carbon graphite, titanium dioxide, latex or cross-linked dextrans such as Sepharose®, cellulose, nylon, cross-linked micelles and Teflon™ may all be used. “Microsphere Detection Guide” from Bangs Laboratories, Fishers Ind. is a helpful guide, which is incorporated herein by reference in its entirety. The beads need not be spherical; irregular particles may be used. In addition, the beads may be porous, thus increasing the surface area of the bead available for either capture probe attachment or tag attachment.

In example embodiments, beads include any bead used for single cell genomics methods as described further herein. Non-limiting examples of beads include hydrogel particles (polyacrylamide, agarose, etc.), colloidal particles (polystyrene, magnetic or polymer particle, etc.), any bead which can leverage phosphoramidate chemistry such as those used in oligonucleotide synthesis known to those skilled in the art (e.g., methylacrylates, polystyrenes, polyacrylamides, polyethylene glycols), paramagnetic beads, and magnetic beads.

In example embodiments, the bead may be a hydrogel particle (see, e.g., Int. Pat. Apl. Pub. No. WO2008/109176 for examples of hydrogel particles, including hydrogel particles containing DNA). Examples of hydrogels include, but are not limited to, agarose or acrylamide-based gels, such as polyacrylamide, poly-N-isopropylacrylamide, or poly N-isopropylpolyacrylamide. For example, an aqueous solution of a monomer may be dispersed in a droplet, and then polymerized, e.g., to form a gel.

In example embodiments, the beads may comprise one or more polymers. Exemplary polymers include, but are not limited to, polystyrene (PS), polycaprolactone (PCL), polyisoprene (PIP), poly(lactic acid), polyethylene, polypropylene, polyacrylonitrile, polyimide, polyamide, and/or mixtures and/or co-polymers of these and/or other polymers. In addition, in some cases, the particles may be magnetic, which could allow for the magnetic manipulation of the particles. For example, the particles may comprise iron or other magnetic materials. The particles could also be functionalized so that they could have other molecules attached, such as proteins, nucleic acids or small molecules. In some embodiments, the particle may be fluorescent.

Beads comprising the spatial barcode nucleic acids of the present disclosure can be obtained by any previously described method. For example, the spatial barcode nucleic acids can be directly synthesized on the beads, such that barcodes can be generated by random synthesis (see, e.g., Macosko et al., 2015, “Highly Parallel Genome-wide Expression Profiling of Individual Cells Using Nanoliter Droplets” Cell 161, 1202-1214; and International patent application number PCT/US2015/049178, published as WO2016/040476 on Mar. 17, 2016). In example embodiments, beads are obtained by 1) performing reverse phosphoramidite synthesis on the surface of the bead to synthesize the 5′ end of the spatial barcode nucleic acids from a linker on the bead; 2) performing reverse phosphoramidite synthesis on the surface of the bead in a pool-and-split fashion, such that in each cycle of synthesis the beads are split into four reactions with one of the four canonical nucleotides (T, C, G, or A) or unique oligonucleotides; 3) repeating this process a large number of times, at least two, and optimally more than twelve, such that, in the latter, there are more than 16 million unique spatial barcodes on the surface of each bead in the pool; and 4) synthesizing or attaching (e.g., ligating) the 3′ end of the spatial barcode nucleic acids comprising a universal sequence for capture by a cell barcode. For synthesis the bead has to be a material that can be maintained during organic synthesis. Non-limiting examples include any bead which can leverage phosphoramidate chemistry such as those used in oligonucleotide synthesis known to those skilled in the art. Direct synthesis of oligonucleotides on beads is a preferred embodiment because direct synthesis allows more diverse barcodes in a more compact sequence.

In another example, the spatial barcode nucleic acids can be synthesized by linking oligonucleotides to beads followed by split-pool hybridization and extension to generate unique cell barcodes for each bead (see, e.g., Klein et al., 2015, “Droplet Barcoding for Single-Cell Transcriptomics Applied to Embryonic Stem Cells” Cell 161, 1187-1201; and International patent application number PCT/US2016/027734, published as WO2016168584A1 on Oct. 20, 2016). In example embodiments, a nucleic acid barcode can be constructed in combinatorial fashion by combining randomly selected indices (for example, about 1, 2, 3, 4, 5, 6, 7, 8, 9, or 10 indexes selected from a pool of sequences for each index). Each such index is a short sequence of nucleotides (for example, DNA, RNA, or a combination thereof) having a distinct sequence. An index can have a length of about, for example, 4, 5, 6, 7, 8, 9, 10, 11, 12, 13, 14, 15, 16, 17, 18, 19, 20, 21, 22, 23, 24, or 25 bp or nt. Accordingly, in some embodiments, the possible barcodes that are used are formed from one or more separate “pools” of barcode elements that are then joined together to produce the final barcode, e.g., using a split-and-pool approach. A pool may contain, for example, at least about 300, at least about 500, at least about 1,000, at least about 3,000, at least about 5,000, or at least about 10,000 distinguishable barcodes. For example, a first pool may contain x₁ elements and a second pool may contain x₂ elements; forming a barcode containing an element from the first pool and an element from the second pool may yield, e.g., x₁x₂ possible barcodes that could be used. It should be noted that x₁ and x₂ may or may not be equal. This process can be repeated any number of times; for example, the barcode may include elements from a first pool, a second pool, and a third pool (e.g., producing x₁x₂x₃ possible barcodes), or from a first pool, a second pool, a third pool, and a fourth pool, etc. Accordingly, due to the potential number of combinations, even a relatively small number of barcode elements can be used to produce a much larger number of distinguishable barcodes.

In another example, the spatial barcode nucleic acids can be synthesized by linking the 5′ end of oligonucleotides containing adaptor sequences to beads to generate functionalized beads followed by emulsion PCR using primers containing unique barcode sequences (see, e.g., Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; and Zilionis, et al., 2017, “Single-cell barcoding and sequencing using droplet microfluidics” Nat Protoc. January; 12 (1):44-73). In this embodiment, each emulsion PCR includes a single primer that can hybridize to oligonucleotides on the functionalized beads and comprise a barcode sequence. Thus, after several rounds of amplification the barcode sequence is transferred to every oligonucleotide on the functionalized beads. This results in beads each having a barcode unique to that bead.

In example embodiments, beads can be suspended in a solution, or they can be located on the surface of a substrate (e.g., arrayed upon the surface of a solid support, such as a glass slide). Art-recognized examples of arrays having beads located on a surface include those wherein beads are located in wells such as a BeadChip array (Illumina Inc., San Diego Calif.), substrates used in sequencing platforms from 454 LifeSciences (a subsidiary of Roche, Basel Switzerland) or substrates used in sequencing platforms from Ion Torrent (a subsidiary of Life Technologies, Carlsbad Calif.). Other solid supports having beads located on a surface are described in U.S. Pat. Nos. 6,266,459; 6,355,431; 6,770,441; 6,859,570; 6,210,891; 6,258,568; or 6,274,320; US Pat. App. Publ. Nos. 2009/0026082 A1; 2009/0127589 A1; 2010/0137143 A1; or 2010/0282617 A1 or PCT Publication No. WO 00/63437, each of which is incorporated herein by reference. Several of the above references describe methods for attaching nucleic acid probes to beads prior to loading the beads in or on a solid support. As such, the collection of beads can include different beads each having a unique (or sufficiently unique and/or near-unique, as described elsewhere herein) probe attached. It will, however, be understood that the beads can be made to include universal primers, and the beads can then be loaded onto an array, thereby forming universal arrays for use in a method set forth herein. The solid supports typically used for bead arrays can be used without beads. For example, nucleic acids, such as probes or primers can be attached directly to the wells or to gel material in wells. Thus, the above references are illustrative of materials, compositions or apparatus that can be modified for use in the methods and compositions set forth herein.

Accordingly, the instant methods can employ an array of beads, wherein different nucleic acid probes are attached to different beads in the array. In this embodiment, each bead can be attached to a different nucleic acid probe and the beads can be randomly distributed on the solid support in order to effectively attach the different nucleic acid probes to the solid support. Optionally, the solid support can include wells having dimensions that accommodate no more than a single bead. In such a configuration, the beads may be attached to the wells due to forces resulting from the fit of the beads in the wells. As described elsewhere herein, it is also possible to use attachment chemistries or capture materials (e.g., a vinyl polymer liquid electrical tape) to adhere or otherwise stably associate the beads with a solid support, optionally including holding the beads in wells that may or may not be present on a solid support.

Nucleic acid probes that are attached to beads can include barcode sequences. A population of the beads can be configured such that each bead is attached to only one type of barcode (e.g., a spatial barcode) and many different beads each with a different barcode are present in the population. In this embodiment, randomly distributing the beads to a solid support will result in randomly locating the nucleic acid probe-presenting beads (and their respective barcode sequences) on the solid support. In some cases, there can be multiple beads with the same barcode sequence such that there is redundancy in the population. However, randomly distributing a redundancy-comprising population of beads on a solid support—especially one that has a capacity that is greater than the number of unique barcodes in the bead population—will tend to result in redundancy of barcodes on the solid support, which will tend to reduce image resolution in the context of the instant disclosure (i.e., where the precise location of a barcoded bead cannot be resolved due to redundancy of barcode use within an arrayed population of beads, it is contemplated that such redundant locations will simply be eliminated from an ultimate image produced by methods of the instant disclosure, or other modes of adjustment (e.g., normalization and/or averaging of values) may also be employed to address such redundancies). Alternatively, in preferred embodiments, the number of different barcodes in a population of beads can exceed the capacity of the solid support in order to produce an array that is not redundant with respect to the population of barcodes on the solid support. The capacity of the solid support will be determined in some embodiments by the number of features (e.g., single-bead occupancy wells) that attach or otherwise accommodate a bead.

A bead or other nucleic acid-presenting solid support of the instant disclosure can include, or can be made by the methods set forth herein to attach, a plurality of different nucleic acid probes. For example, a bead or other nucleic acid-presenting solid support can include at least 10, 100, 1×10³, 1×10⁴, 1×10⁵, 1×10⁶, 1×10⁷, 1×10⁸, 1×10⁹ or more different probes. Alternatively or additionally, a bead or other nucleic acid-presenting solid support can include at most 1×10⁹, 1×10⁸, 1×10⁷, 1×10⁶, 1×10⁵, 1×10⁴, 1×10³, 100, or fewer different probes. It will be understood that each of the different probes can be present in several copies, for example, when the probes have been amplified to form a cluster. Thus, the above ranges can describe the number of different nucleic acid clusters on a bead or other nucleic acid-presenting solid support of the instant disclosure. It will also be understood that the above ranges can describe the number of different barcodes, target capture sequences, or other sequence elements set forth herein as being unique (or sufficiently unique) to particular nucleic acid probes. Alternatively or additionally, the ranges can describe the number of extended probes or modified probes created on a bead or other nucleic acid-presenting solid support of the instant disclosure using a method set forth herein.

Features may be present on a bead or other solid support of the instant disclosure prior to contacting the bead or other solid support with nucleic acid probes. For example, in embodiments where probes are attached to a bead or other solid support via hybridization to primers, the primers can be attached at the features, whereas interstitial areas outside of the features substantially lack any of the primers. Nucleic acid probes can be captured at preformed features on a bead or other solid support, and optionally amplified on the bead or other solid support, e.g., using methods set forth in U.S. Pat. Nos. 8,895,249 and 8,778,849 and/or U.S. Patent Publication No. 2014/0243224 A1, each of which is incorporated herein by reference. Alternatively, a bead or other solid support may have a lawn of primers or may otherwise lack features. In this case, a feature can be formed by virtue of attachment of a nucleic acid probe on the bead or other solid support. Optionally, the captured nucleic acid probe can be amplified on the bead or other solid support such that the resulting cluster becomes a feature. Although attachment is exemplified above as capture between a primer and a complementary portion of a probe, it will be understood that capture moieties other than primers can be present at pre-formed features or as a lawn. Other exemplary capture moieties include, but are not limited to, chemical moieties capable of reacting with a nucleic acid probe to create a covalent bond or receptors capable of binding non-covalently to a ligand on a nucleic acid probe.

A step of attaching nucleic acid probes to a bead or other solid support can be carried out by providing a fluid that contains a mixture of different nucleic acid probes and contacting this fluidic mixture with the bead or other solid support. The contact can result in the fluidic mixture being in contact with a surface to which many different nucleic acid probes from the fluidic mixture will attach. Thus, the probes have random access to the surface (whether the surface has pre-formed features configured to attach the probes or a uniform surface configured for attachment). Accordingly, the probes can be randomly located on the bead or other solid support.

The total number and variety of different probes that end up attached to a surface can be selected for a particular application or use. For example, in embodiments where a fluidic mixture of different nucleic acid probes is contacted with a bead or other solid support for purposes of attaching the probes to the support, the number of different probe species can exceed the occupancy of the bead or other solid support for probes. Thus, the number and variety of different probes that attach to the bead or other solid support can be equivalent to the probe occupancy of the bead or other solid support.

Alternatively, the number and variety of different probe species on the bead or other solid support can be less than the occupancy (i.e., there will be redundancy of probe species such that the bead or other solid support may contain multiple features having the same probe species). Such redundancy can be achieved, for example, by contacting the bead or other solid support with a fluidic mixture that contains a number and variety of probe species that is substantially lower than the probe occupancy of the bead or other solid support.

Attachment of the nucleic acid probes can be mediated by hybridization of the nucleic acid probes to complementary primers that are attached to the bead or other solid support, chemical bond formation between a reactive moiety on the nucleic acid probe and the bead or other solid support (examples are set forth in U.S. Pat. Nos. 8,895,249 and 8,778,849, and in U.S. Patent Publication No. 2014/0243224 A1, each of which is incorporated herein by reference), affinity interactions of a moiety on the nucleic acid probe with a bead- or other solid support-bound moiety (e.g. between known receptor-ligand pairs such as streptavidin-biotin, antibody-epitope, lectin-carbohydrate and the like), physical interactions of the nucleic acid probes with the bead or other solid support (e.g. hydrogen bonding, ionic forces, van der Waals forces and the like), or other interactions known in the art to attach nucleic acids to surfaces.

In some embodiments, attachment of a nucleic acid probe is non-specific with regard to any sequence differences between the nucleic acid probe and other nucleic acid probes that are or will be attached to the bead or other solid support. For example, different probes can have a universal sequence that complements surface-attached primers, or the different probes can have a common moiety that mediates attachment to the surface. Alternatively, each of the different probes (or a subpopulation of different probes) can have a unique (or sufficiently unique) sequence that complements a unique (or sufficiently unique) primer on the bead or other solid support, or they can have a unique (or sufficiently unique) moiety that interacts with one or more different reactive moiety on the bead or other solid support. In such cases, the unique (or sufficiently unique) primers or unique (or sufficiently unique) moieties can, optionally, be attached at predefined locations in order to selectively capture particular probes, or particular types of probes, at the respective predefined locations.

One or more features on a bead or other solid support can each include a single molecule of a particular probe. The features can be configured, in some embodiments, to accommodate no more than a single nucleic acid probe molecule. However, whether or not the feature can accommodate more than one nucleic acid probe molecule, the feature may nonetheless include no more than a single nucleic acid probe molecule. Alternatively, an individual feature can include a plurality of nucleic acid probe molecules, for example, an ensemble of nucleic acid probe molecules having the same sequence as each other. In particular embodiments, the ensemble can be produced by amplification from a single nucleic acid probe template to produce amplicons, for example, as a cluster attached to the surface.

A method set forth herein can use any of a variety of amplification techniques. Exemplary techniques that can be used include, but are not limited to, polymerase chain reaction (PCR), rolling circle amplification (RCA), multiple displacement amplification (MDA), or random prime amplification (RPA). In some embodiments the amplification can be carried out in solution, for example, when features of an array are capable of containing amplicons in a volume having a desired capacity. In certain embodiments, an amplification technique used in a method of the present disclosure will be carried out on solid phase. For example, one or more primer species (e.g., universal primers for one or more universal primer binding site present in a nucleic acid probe) can be attached to a bead or other solid support. In PCR embodiments, one or both of the primers used for amplification can be attached to a bead or other solid support (e.g., via a gel). Formats that utilize two species of primers attached to a bead or other solid support are often referred to as bridge amplification because double stranded amplicons form a bridge-like structure between the two surface attached primers that flank the template sequence that has been copied. Exemplary reagents and conditions that can be used for bridge amplification are described, for example, in U.S. Pat. Nos. 5,641,658; 7,115,400; and 8,895,249; and/or U.S. Patent Publication Nos. 2002/0055100 A1, 2004/0096853 A1, 2004/0002090 A1, 2007/0128624 A1 and 2008/0009420 A1, each of which is incorporated herein by reference. Solid-phase PCR amplification can also be carried out with one of the amplification primers attached to a bead or other solid support and the second primer in solution. An exemplary format that uses a combination of a surface attached primer and soluble primer is the format used in emulsion PCR as described, for example, in Dressman et al., Proc. Natl. Acad. Sci. USA 100:8817-8822 (2003), WO 05/010145, or U.S. Patent Publication Nos. 2005/0130173 A1 or 2005/0064460 A1, each of which is incorporated herein by reference. Emulsion PCR is illustrative of the format, and it will be understood that for purposes of the methods set forth herein the use of an emulsion is optional and indeed for several embodiments an emulsion is not used.

RCA techniques can be modified for use in a method of the present disclosure. Exemplary components that can be used in an RCA reaction and principles by which RCA produces amplicons are described, for example, in Lizardi et al., Nat. Genet. 19:225-232 (1998) and U.S. Patent Publication No. 2007/0099208 A1, each of which is incorporated herein by reference. Primers used for RCA can be in solution or attached to a bead or other solid support. The primers can be one or more of the universal primers described herein.

MDA techniques can be modified for use in a method of the present disclosure. Some basic principles and useful conditions for MDA are described, for example, in Dean et al., Proc Natl. Acad. Sci. USA 99:5261-66 (2002); Lage et al., Genome Research 13:294-307 (2003); Walker et al., Molecular Methods for Virus Detection, Academic Press, Inc., 1995; Walker et al., Nucl. Acids Res. 20:1691-96 (1992); U.S. Pat. Nos. 5,455,166; 5,130,238; and 6,214,587, each of which is incorporated herein by reference. Primers used for MDA can be in solution or attached to a bead or other solid support at an amplification site. Again, the primers can be one or more of the universal primers described herein.

In particular embodiments a combination of the above-exemplified amplification techniques can be used. For example, RCA and MDA can be used in a combination wherein RCA is used to generate a concatameric amplicon in solution (e.g., using solution-phase primers). The amplicon can then be used as a template for MDA using primers that are attached to a bead or other solid support (e.g., universal primers). In this example, amplicons produced after the combined RCA and MDA steps will be attached to the bead or other solid support.

In certain aspects of the instant disclosure, a capture material is employed to associate a bead array with a solid support (e.g., a glass slide). In some embodiments, the capture material is a liquid electrical tape. An exemplary liquid electrical tape of the instant disclosure is Permatex™ liquid electrical tape, which is a weatherproof protectant for wiring and electrical connections. Liquid capture material such as liquid tape can be applied as a liquid, which then dries to a vinyl polymer that resists dirt, dust, chemicals, and moisture. Capture materials of the instant disclosure can be applied by any of a number of methods, including brushed onto the solid support, sprayed onto the solid support, or the like, or via submersion of the solid support in the capture material. For certain forms of liquid capture material, use of a brush top applicator can allow coverage without gaps and can enable access to tight spaces, which offers advantages in certain embodiments over forms of capture material (i.e., tape) that are applied in a non-liquid state.

While liquid electrical tape has been exemplified as a capture material for use in the methods and compositions of the instant disclosure, other capture materials are also contemplated for such use, including any art-recognized glue or other reagent that is (a) spreadable and/or depositable upon a solid surface (e.g., upon a slide, optionally a slide that allows for light transmission through the slide, e.g., a microscope slide) and (b) capable of binding or otherwise capturing a population of beads of 1-100 μm size. Exemplary other capture materials that are expressly contemplated include latex such as cis-1,4-polyisoprene and other rubbers, as well as elastomers (which are generally defined as polymers that possess viscoelasticity (i.e., both viscosity and elasticity), very weak inter-molecular forces, and generally low Young's modulus and high failure strain compared with other materials), including artificial elastomers (e.g., neoprene) and/or silicone elastomers. Acrylate polymers (e.g., scotch tape) are also expressly contemplated, e.g., for use as a capture material of the instant disclosure. Exemplary other capture materials that are expressly contemplated include super glue adhesives (e.g., Loctite® super glue), gels (e.g. agarose, acrylamide), rubberized sealant sprays (e.g., GLORILLA® glue, PLASTI DIP® multipurpose rubber coating), silicone conformal coating (e.g., Fine-L-Kote SR), or other conformal coatings (e.g., Urethane resin, Epoxy resin, Acrylic resin, Parylene). Additionally, bead oligos may be transferred to agarose or acrylamide gel substrates for subsequent release into tissues.

In example embodiments, the array is printed on a solid support. In one embodiment, the printed spatial barcode nucleic acids are amplified on the solid support as described herein (e.g., bridge amplification). In example embodiments, the solid support is a slide or an array on a slide. As used herein the term “slide” includes an “array”, “substrate” or “surface” including a plurality of spatial barcode nucleic acids as described herein. For the spatial array-based analytical methods described herein, a substrate functions as a support for direct or indirect attachment of spatial barcode nucleic acids to features of the array. In addition, in some embodiments, a substrate (e.g., the same substrate or a different substrate) can be used to provide support to a biological sample, particularly, for example, a thin tissue section. Accordingly, a “substrate” is a support that is insoluble in aqueous liquid and which allows for positioning of biological samples, analytes, features, and/or spatial barcode nucleic acids on the substrate.

Further, a “substrate” as used herein, and when not preceded by the modifier “chemical”, refers to a member with at least one surface that generally functions to provide physical support for biological samples, analytes, and/or any of the other chemical and/or physical moieties, agents, and structures described herein. Substrates can be formed from a variety of solid materials, gel-based materials, colloidal materials, semi-solid materials (e.g., materials that are at least partially cross-linked), materials that are fully or partially cured, and materials that undergo a phase change or transition to provide physical support. Examples of substrates that can be used in the methods and systems described herein include, but are not limited to, slides (e.g., slides formed from various glasses, slides formed from various polymers), hydrogels, layers and/or films, membranes (e.g., porous membranes), flow cells, cuvettes, wafers, plates, or combinations thereof. In some embodiments, substrates can optionally include functional elements such as recesses, protruding structures, microfluidic elements (e.g., channels, reservoirs, electrodes, valves, seals), and various markings. Slides and arrays for spatial profiling have been described (see, e.g., Visium Spatial Capture Technology, 10×Genomics, Pleasanton, CA; WO2020047007A2; WO2020123317A2; WO2020047005A1; WO2020176788A1; and WO2020190509A9). The capture probes comprising spatial barcodes can be replaced with the spatial barcode nucleic acids comprising spatial barcodes as described herein.

Slides comprising spatial barcode nucleic acids can be obtained by synthesizing spatial barcode nucleic acids and attaching them to a slide or array. In an example embodiment, spatial barcode nucleic acids are added to specific locations of an array.

Arrays can be prepared by depositing features (e.g., droplets, beads) on a substrate surface to produce a spatially-barcoded array. Methods of depositing (e.g., droplet manipulation) features are known in the art (see, U.S. Patent Application Publication No. 2008/0132429; Rubina, A. Y., et al., Biotechniques.2003 May; 34(5):1008-14, 1016-20, 1022; and Vasiliskov et al. Biotechniques. 1999 September; 27(3):592-4, 596-8, 600 passim). A feature can be printed or deposited at a specific location on the substrate (e.g., inkjet printing). In some embodiments, each feature can have a spatial barcode nucleic acid. In some embodiments, a feature can be printed or deposited at the specific location using an electric field. A feature can contain a photo-crosslinkable polymer precursor and an oligonucleotide. In some embodiments, the photo-crosslinkable polymer precursor can be deposited into a patterned feature on the substrate (e.g., well). A “photo-crosslinkable polymer precursor” refers to a compound that cross-links and/or polymerizes upon exposure to light. In some embodiments, one or more photoinitiators may also be included to induce and/or promote polymerization and/or cross-linking (see, e.g., Choi et al. Biotechniques. 2019 January; 66(1):40-53).

Arrays can be prepared by a variety of methods. In some embodiments, arrays are prepared through the synthesis (e.g., in situ synthesis) of oligonucleotides on the array, or by jet printing or lithography. In example embodiments, spatial barcode nucleic acids are deterministically patterned via synthesis. For example, light-directed synthesis of high-density DNA oligonucleotides can be achieved by photolithography or solid-phase DNA synthesis. To implement photolithographic synthesis, synthetic linkers modified with photochemical protecting groups can be attached to a substrate and the photochemical protecting groups can be modified using a photolithographic mask (applied to specific areas of the substrate) and light, thereby producing an array having localized photo-deprotection. Many of these methods are known in the art, and are described e.g., in Miller et al., “Basic concepts of microarrays and potential applications in clinical microbiology.” Clinical Microbiology Reviews 22.4(2009):611-633; US201314111482A; U.S. Pat. No. 9,593,365B2; US2019203275; and WO2018091676.

### Tissues

In some embodiments, a tissue section is employed. The tissue can be derived from a multicellular organism. Exemplary multicellular organisms include, but are not limited to a mammal, plant, algae, nematode, insect, fish, reptile, amphibian, fungi or Plasmodium falciparum. Exemplary species are set forth previously herein or known in the art. The tissue can be freshly excised from an organism, or it may have been previously preserved for example by freezing, embedding in a material such as paraffin (e.g. formalin fixed paraffin embedded samples (FFPE)), formalin fixation, infiltration, dehydration or the like. Optionally, a tissue section can be cryosectioned, using techniques and compositions as described herein and as known in the art. As a further option, a tissue can be permeabilized to allow nuclei to be accessible by spatial barcode nucleic acids. As used herein, the term “tissue” is intended to mean an aggregation of cells, and, optionally, intercellular matter. Typically, the cells in a tissue are not free floating in solution and instead are attached to each other to form a multicellular structure. Exemplary tissue types include muscle, nerve, epidermal and connective tissues. Tissues can also be from a diseased subject, such as but not limited to an autoimmune disease or cancer (e.g., a tissue from irritable bowel disease (IBD) or MS, or a tumor tissue).

A method of the present disclosure can include a step of contacting a biological specimen (i.e., a cryosectioned tissue sample) with a bead or other solid support that has spatial barcode nucleic acids attached thereto. In some embodiments, the spatial barcode nucleic acids are randomly located on the bead or other solid support. The identity and location of the spatial barcode nucleic acids may have been decoded prior to contacting the biological specimen with the bead or other solid support. Alternatively, the identity and location of the spatial barcode nucleic acids can be determined after contacting the bead or other solid support with the biological specimen.

As used herein, the term “cryosection” refers to a piece of tissue, e.g. a biopsy, that has been obtained from a subject, snap frozen, embedded in optimal cutting temperature embedding material, frozen, and cut into thin sections. In certain embodiments, the thin sections can be directly applied to an array of beads captured upon a solid support (e.g., a slide), or the thin sections can be fixed (e.g. in methanol or paraformaldehyde) and applied to a bead-presenting planar surface, e.g., a slide upon which a layer of microbeads has been attached/arrayed.

In example embodiments, the tissue can be obtained from a complex multicellular system (e.g., organoid, tissue explant, or organ on a chip) (see, e.g., Yin X, Mead B E, Safaee H, Langer R, Karp J M, Levy O. Engineering Stem Cell Organoids. Cell Stem Cell. 2016; 18(1):25-38; Clevers, Modeling Development and Disease with Organoids, Cell. 2016 Jun. 16; 165(7):1586-1597; Porter, R. J., Murray, G. I. & McLean, M. H. Current concepts in tumour-derived organoids. Br J Cancer 123, 1209-1218 (2020). doi.org/10.1038/s41416-020-0993-5; Sontheimer-Phelps, A., Hassell, B. A. & Ingber, D. E. Modelling cancer in microfluidic human organs-on-chips. Nat. Rev. Cancer 19, 65-81 (2019); and Wu, Q., Liu, J., Wang, X. et al. Organ-on-a-chip: recent breakthroughs and future prospects. BioMed Eng OnLine 19, 9 (2020); Ingber, D. E. Developmentally inspired human ‘organs on chips’. Development 145, pii: dev156125 (2018); Ghosh S, Prasad M, Kundu K, et al. Tumor Tissue Explant Culture of Patient-Derived Xenograft as Potential Prioritization Tool for Targeted Therapy. Front Oncol. 2019; 9:17; Neil J E, Brown M B, Williams A C. Human skin explant model for the investigation of topical therapeutics. Sci Rep. 2020; 10(1):21192; and Grivel J C, Margolis L. Use of human tissue explants to study human infectious agents. Nat Protoc. 2009; 4(2):256-269). Tissues or complex multicellular systems include a patient derived organoid (PDO) or patient derived xenograft (PDX).

A tissue can be prepared in any convenient or desired way for its use in a method, composition or apparatus herein. Fresh, frozen, fixed or unfixed tissues can be used. A tissue can be fixed or embedded using methods described herein or known in the art.

A tissue sample for use herein, can be fixed by deep freezing at temperature suitable to maintain or preserve the integrity of the tissue structure, e.g., less than −20° C. In another example, a tissue can be prepared using formalin-fixation and paraffin embedding (FFPE) methods which are known in the art. Other fixatives and/or embedding materials can be used as desired. A fixed or embedded tissue sample can be sectioned, i.e. thinly sliced, using known methods. For example, a tissue sample can be sectioned using a chilled microtome or cryostat, set at a temperature suitable to maintain both the structural integrity of the tissue sample and the chemical properties of the nucleic acids in the sample. Exemplary additional fixatives that are expressly contemplated include alcohol fixation (e.g., methanol fixation, ethanol fixation), glutaraldehyde fixation and paraformaldehyde fixation.

In some embodiments, a tissue sample will be treated to remove embedding material (e.g., to remove paraffin or formalin) from the sample prior to tagging nuclei. This can be achieved by contacting the sample with an appropriate solvent (e.g., xylene and ethanol washes). Treatment can occur prior to contacting the tissue sample with a spatial array as set forth herein or the treatment can occur while the tissue sample is on the solid support-captured bead array.

Exemplary methods for manipulating tissues for use with solid supports to which nucleic acids are attached are set forth in US Pat. App. Publ. No. 2014/0066318 A1, which is incorporated herein by reference.

The thickness of a tissue sample or other biological specimen that is contacted with a bead array in a method, composition or apparatus set forth herein can be any suitable thickness desired. In representative embodiments, the thickness will be at least 0.1 μm, 0.25 μm, 0.5 μm, 0.75 μm, 1 μm, 5 μm, 10 μm, 50 μm, 100 μm or thicker. Alternatively or additionally, the thickness of a tissue sample that is contacted with bead array will be no more than 100 μm, 50 μm, 10 μm, 5μ, 1μ, 0.5 μm, 0.25 μm, 0.1 μm or thinner.

A particularly relevant source for a tissue sample is a human being. Another source of a tissue sample is an animal model (e.g., a mouse tissue sample). The sample can be derived from an organ, including for example, an organ of the central nervous system such as brain, brainstem, cerebellum, spinal cord, cranial nerve, or spinal nerve; an organ of the musculoskeletal system such as muscle, bone, tendon or ligament; an organ of the digestive system such as salivary gland, pharynx, esophagus, stomach, small intestine, large intestine, liver, gallbladder or pancreas; an organ of the respiratory system such as larynx, trachea, bronchi, lungs or diaphragm; an organ of the urinary system such as kidney, ureter, bladder or urethra; a reproductive organ such as ovary, fallopian tube, uterus, vagina, placenta, testicle, epididymis, vas deferens, seminal vesicle, prostate, penis or scrotum; an organ of the endocrine system such as pituitary gland, pineal gland, thyroid gland, parathyroid gland, or adrenal gland; an organ of the circulatory system such as heart, artery, vein or capillary; an organ of the lymphatic system such as lymphatic vessel, lymph node, bone marrow, thymus or spleen; a sensory organ such as eye, ear, nose, or tongue; or an organ of the integument such as skin, subcutaneous tissue or mammary gland. In some embodiments, a tissue sample is obtained from a bodily fluid or excreta, such as blood, lymph, tears, sweat, saliva, semen, vaginal secretion, ear wax, fecal matter or urine.

A sample from a human can be considered (or suspected) healthy or diseased when used. In some cases, two samples can be used: a first being considered diseased and a second being considered as healthy (e.g., for use as a healthy control). Any of a variety of conditions can be evaluated, including but not limited to, an autoimmune disease, cancer, cystic fibrosis, aneuploidy, pathogenic infection, psychological condition, hepatitis, diabetes, sexually transmitted disease, heart disease, stroke, cardiovascular disease, multiple sclerosis (MS) or muscular dystrophy. Certain contemplated conditions include genetic conditions or conditions associated with pathogens having identifiable genetic signatures.

In example embodiments, tissue is prepared for nuclei tagging using steps from single nuclei sequencing methods (e.g., snRNA-seq) (see, e.g., Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Methods. Nat 2017 October; 14(10):955-958; International Patent Application No. PCT/US2016/059239, published as WO2017164936 on Sep. 28, 2017; International Patent Application No. PCT/US2018/060860, published as WO/2019/094984 on May 16, 2019; International Patent Application No. PCT/US2019/055894, published as WO/2020/077236 on Apr. 16, 2020; Drokhlyansky, et al., “The enteric nervous system of the human and mouse colon at a single-cell resolution,” bioRxiv 746743; doi: doi.org/10.1101/746743; Drokhlyansky E, Smillie C S, Van Wittenberghe N, et al. The Human and Mouse Enteric Nervous System at Single-Cell Resolution. Cell. 2020; 182(6):1606-1622.e23; and Slyper, M., Porter, C. B. M., Ashenberg, O. et al. (2020). A single-cell and single-nucleus RNA-seq toolbox for fresh and frozen human tumors. Nature Medicine 26(5):792-802). In example embodiments, methods for preparing and isolating nuclei that preserve the nuclear envelope and ribosomes (RAISIN (Ribosomes And Intact SIngle Nucleus) RNA-seq) or that preserve the both the rough ER and its attached ribosomes on the outer nuclear membrane (INNER Cell (INtact Nucleus and Endoplasmic Reticulum from a single Cell) RNA-seq) are applicable to tagged nuclei (see, WO/2020/077236).

In an example embodiment, single nuclei are prepared from FFPE tissue sections (see, e.g., WO/2020/077236). For example, an FFPE tissue sample is processed by dissolving paraffin in a solvent, preferably the solvent is selected from xylene or mineral oil, wherein the tissue is dissolved at a temperature between 4 C to 90 C, preferably room temperature (20 to 25 C) or 90 C; rehydrating the tissue using a gradient of ethanol from 100% to 0% ethanol (EtOH); transferring the rehydrated tissue to a volume of a first buffer comprising a buffering agent, a detergent and an ionic strength between 100 mM and 200 mM, optionally the first buffer comprises protease inhibitors or proteases and/or BSA. In an example embodiment, single nuclei are prepared from tissue samples (see, e.g., Raisin-seq) using buffer comprising 10 mM Tris, 0.49% CHAPS, 146 mM NaCl, 1 mM CaCl₂, 21 mM MgCl₂, and 0.01% BSA (CST). In an example embodiment, single nuclei are prepared from tissue samples (see, e.g., INNER Cell-seq) using buffer comprising 10 mM Tris, 0.03% Tween-20, 146 mM NaCl, 1 mM CaCl₂, 21 mM MgCl₂, and 0.01% BSA (TST). In one example embodiment, the buffers are used during extraction of nuclei from the tissue.

In some embodiments, a biological sample can be permeabilized to facilitate transfer of spatial barcode nucleic acids into the sample. If a sample is not permeabilized sufficiently, the amount of spatial barcode nucleic acids into the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™, Tween-20™, or sodium dodecyl sulfate (SDS)), and enzymes (e.g., trypsin, proteases (e.g., proteinase K). In some embodiments, the detergent is an anionic detergent (e.g., SDS or N-lauroylsarcosine sodium salt solution). In some embodiments, the biological sample can be permeabilized using any of the methods described herein (e.g., using any of the detergents described herein, e.g., SDS and/or N-lauroylsarcosine sodium salt solution) before or after enzymatic treatment (e.g., treatment with any of the enzymes described herein, e.g., trypsin, proteases (e.g., pepsin and/or proteinase K)). Additional methods for sample permeabilization are described, for example, in Jamur et al., Method Mol. Biol. 588:63-66, 2010, the entire contents of which are incorporated herein by reference.

### Cleaving Linkers and Delivering Spatial Barcodes to Nuclei

In example embodiments, the spatial barcode nucleic acids in contact with the permeabilized tissue sample are delivered to the nuclei. In example embodiments, the tissue sample is incubated in a dissociation buffer as described herein (see, Example 1 Dissociation Buffer (DB)). In example embodiments, the incubation is at 4-25° C., preferably about 4° C. The linkers are then cleaved (e.g., using light, chemical or an enzyme) to release the spatial barcode nucleic acids from the spatial array. In example embodiments, the spatial barcode nucleic acids are delivered to the nuclei by diffusion. In example embodiments, the spatial barcode nucleic acids are delivered to the nuclei using electroporation. With electroporation, spatial barcode nucleic acids can enter a cell through one or more pores in the cellular membrane formed by applied electricity. The pore of the membrane can be reversible based on the applied field strength and pulse duration. In example embodiments, the spatial barcode nucleic acids are delivered to the nuclei using sonoporation. Cell membranes can be temporarily permeabilized using sound waves, allowing cellular uptake of spatial barcode nucleic acids. In example embodiments, the spatial barcode nucleic acids are delivered to the nuclei using hydroporation. Spatial barcode nucleic acids can be delivered to cells via hydrodynamic pressure.

Isolating Tagged Nuclei from Tissue

In example embodiments, nuclei are extracted from the tagged tissue by dissociating the tissue with extraction buffer (see, e.g., Example 1, Extraction Buffer (ExB)). In example embodiments, the extraction buffer includes a detergent. In example embodiments, the detergent is Triton X-100. In example embodiments, the detergent is CHAPS (3-[(3-cholamidopropyl)dimethylammonio]-1-propanesulfonate). In example embodiments, the detergent is Tween-20. In one embodiment, the tissue is dissociated by pipetting. In one embodiment, the tissue is dissociated by gentle scraping. In example embodiments, the tagged nuclei can be sorted by FACS in the case where the spatial barcode nucleic acids include a fluorescent dye. Thus, only tagged nuclei will be used in the following steps. Sorting nuclei can allow the same efficiency as using untagged nuclei.

### Single Cell Genomics Assays

Single-cell omics sequencing was first achieved for the transcriptome in 2009, which was followed by fast development of technologies for profiling the genome, DNA methylome, 3D genome architecture, chromatin accessibility, histone modifications, etc., in an individual cell (see, e.g., Wen L, Tang F. Recent advances in single-cell sequencing technologies. Precis Clin Med. 2022; 5 (1):pbac002. Published 2022 Jan. 31). In example embodiments, the tagged nuclei generated by the present methods can be used with any of these single cell genomics assays to make the assays spatially resolved.

In example embodiments, single cell genomics assays generally include barcodes used to identify the cell of origin of the genomic analyte (i.e., cell barcodes). For example, the analyte can be RNA, such as a poly-A tailed mRNA, a genomic DNA sequence, joined DNA fragments, immunoprecipitated genomic DNA, or antibody specific oligonucleotides. The cell barcodes also generally are associated with a unique molecular identifier (UMI), such that the number of each analyte for each single cell can be counted (i.e., quantified). The term “unique molecular identifiers” (UMI) as used herein refers to a sequencing linker or a subtype of nucleic acid barcode used in a method that uses molecular tags to detect and quantify unique amplified products (see e.g., Islam S. et al., 2014. Nature Methods No: 11, 163-166). In example embodiments, a UMI is a random sequence of between 4 and 20 bases. A UMI is used to distinguish effects through a single clone from multiple clones (e.g., an amplified product from a single clone will include the same UMI as the original clone). The term “clone” as used herein may refer to a single mRNA or target nucleic acid to be sequenced. Thus, the UMI may also be used to determine the number of a specific analyte or events that gave rise to an amplified product. In an example embodiment, each cell barcode for a single nuclei is the same, but each UMI associated with the cell barcode is randomized, such that each capture event has a unique UMI. For example, a spatial barcode nucleic acid can be captured by a cell barcode nucleic acid that includes a UMI. The number of different UMIs sequenced for a spatial barcode in a single nuclei identified by a cell barcode would indicate the number of spatial barcodes that the single nuclei was tagged with (e.g., counting UMIs for sequence reads having the same cell barcode, but different spatial barcodes).

In example embodiments, nucleic acid sequences comprising a cell barcode and UMI can capture more than one spatial barcode if the single cell was in proximity to or overlapping more than one location on the array. In example embodiments, computational methods can be used to resolve the location of single cells in the spatial array, such as by counting UMIs. The computational methods can use the distance on the array of the different spatial barcodes. If the spatial barcodes are close on the array the location is most likely somewhere in between or overlapping the locations. If the spatial barcodes are far way then most likely the spatial barcode nucleic acids diffused away from their location and the location is the spatial barcode with the most UMIs because the spatial barcode with higher UMIs was the predominant spatial barcode that diffused into that single cell.

Single cell assays generally add the cell barcode by one of three methods; adding single cells/nuclei to individual wells each having a unique barcode or combination of barcodes; pool and split indexing of intact nuclei/cells; and segregating single cells/nuclei into separate reaction vessels that include unique cell barcodes, such as microwells, reaction chambers, or droplets. In an example embodiment, for pool and split indexing, the spatial barcode nucleic acid is modified to include a common universal sequence (e.g., handle sequence) present on each target analyte, such that both the spatial barcode and analyte can capture the same index sequence at each pool and split step. In an example embodiment, for droplet methods, the spatial barcode nucleic acid is modified to include a common universal sequence present on each target analyte, such that both can be captured by a cell barcode nucleic acid present in each droplet (e.g., usually on a bead). Single cell assays generally use a capture sequence specific to an analyte, such as an analyte having a poly-A tail or a specific adapter sequence added by a transposase (e.g., tagmentation adapter). These sequences can easily be included in a spatial barcode nucleic acid.

The method of the present disclosure can be adapted, such that the spatial barcode can be captured by the cell barcode for any single cell assay. Specific examples are provided below.

### Spatially Resolved Single Nucleus/Cell RNA-Sequencing

In example embodiments, the single cell genomics sequencing library is a single cell RNA sequencing library (see, e.g., Trombetta, J. J., Gennert, D., Lu, D., Satija, R., Shalek, A. K. & Regev, A. Preparation of Single-Cell RNA-Seq Libraries for Next Generation Sequencing. Curr Protoc Mol Biol. 107, 4 22 21-24 22 17, doi: 10.1002/0471142727.mb0422s107 (2014); Qi Z, Barrett T, Parikh A S, Tirosh I, Puram S V. Single-cell sequencing and its applications in head and neck cancer. Oral Oncol. 2019; 99:104441; Kalisky, T., Blainey, P. & Quake, S. R. Genomic Analysis at the Single-Cell Level. Annual review of genetics 45, 431-445, (2011); Kalisky, T. & Quake, S. R. Single-cell genomics. Nature Methods 8, 311-314 (2011); Islam, S. et al. Characterization of the single-cell transcriptional landscape by highly multiplex RNA-seq. Genome Research, (2011); Tang, F. et al. RNA-Seq analysis to capture the transcriptome landscape of a single cell. Nature Protocols 5, 516-535, (2010); Tang, F. et al. mRNA-Seq whole-transcriptome analysis of a single cell. Nature Methods 6, 377-382, (2009); Ramskold, D. et al. Full-length mRNA-Seq from single-cell levels of RNA and individual circulating tumor cells. Nature Biotechnology 30, 777-782, (2012); and Hashimshony, T., Wagner, F., Sher, N. & Yanai, I. CEL-Seq: Single-Cell RNA-Seq by Multiplexed Linear Amplification. Cell Reports, Cell Reports, Volume 2, Issue 3, p 666-673, 2012). PMCID: 4338574).

In example embodiments, the disclosure involves plate based single cell RNA sequencing (see, e.g., Picelli, S. et al., 2014, “Full-length RNA-seq from single cells using Smart-seq2” Nature protocols 9, 171-181, doi: 10.1038/nprot.2014.006). In example embodiments, the disclosure involves high-throughput single-cell RNA-seq. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. January; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Rosenberg et al., “Single-cell profiling of the developing mouse brain and spinal cord with split-pool barcoding” Science 15 Mar. 2018; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; Gierahn et al., “Seq-Well: portable, low-cost RNA sequencing of single cells at high throughput” Nature Methods 14, 395-398 (2017); and Hughes, et al., “Highly Efficient, Massively-Parallel Single-Cell RNA-Seq Reveals Cellular States and Molecular Features of Human Skin Pathology” bioRxiv 689273; doi: doi.org/10.1101/689273, all the contents and disclosure of each of which are herein incorporated by reference in their entirety.

In example embodiments, the disclosure involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958; International Patent Application No. PCT/US2016/059239, published as WO2017164936 on Sep. 28, 2017; International Patent Application No. PCT/US2018/060860, published as WO/2019/094984 on May 16, 2019; International Patent Application No. PCT/US2019/055894, published as WO/2020/077236 on Apr. 16, 2020; Drokhlyansky, et al., “The enteric nervous system of the human and mouse colon at a single-cell resolution,” bioRxiv 746743; doi: doi.org/10.1101/746743; and Drokhlyansky E, Smillie C S, Van Wittenberghe N, et al. The Human and Mouse Enteric Nervous System at Single-Cell Resolution. Cell. 2020; 182(6):1606-1622.e23, which are herein incorporated by reference in their entirety.

Single cell/nuclei RNA-seq generally uses a poly-T capture sequence and reverse transcription (RT) to capture mRNAs having a poly-A tail. In an example embodiment, tagged nuclei are generated according to the present disclosure with spatial barcode nucleic acids comprising a poly-A sequence. The tagged nuclei are then used in any RNA-seq method above. For example tagged nuclei can be used in a pool and split method or can be segregated to individual wells or droplets. The spatial barcode nucleic acids and mRNA are both captured by a cell barcode sequence. For plate or microwell based methods, reverse transcription and use of a template switching primer can add a unique barcode to mRNA and the spatial barcode nucleic acid.

### Spatially Resolved Single Cell Chromatin Accessibility

In example embodiments, the single cell genomics sequencing library is a single cell Assay for Transposase Accessible Chromatin using sequencing (ATAC-seq) sequencing library. ATAC-seq can be used to identified accessible chromatin in a cell (see, e.g., Buenrostro, et al., Transposition of native chromatin for fast and sensitive epigenomic profiling of open chromatin, DNA-binding proteins and nucleosome position. Nature methods 2013; 10(12):1213-1218). Using plate-, droplet-, or combinatorial indexing-based methods, thousands to hundreds of thousands of individual cells/nuclei can be analyzed in a single sample (see, e.g., Buenrostro et al., Single-cell chromatin accessibility reveals principles of regulatory variation. Nature 523, 486-490 (2015); Cusanovich, D. A., Daza, R., Adey, A., Pliner, H., Christiansen, L., Gunderson, K. L., Steemers, F. J., Trapnell, C. & Shendure, J. Multiplex single-cell profiling of chromatin accessibility by combinatorial cellular indexing. Science. 2015 May 22; 348(6237):910-4. doi: 10.1126/science.aab1601. Epub 2015 May 7; Cusanovich D A, Hill A J, Aghamirzaie D, et al. A Single-Cell atlas of in vivo mammalian chromatin accessibility. Cell. 2018; 174:1309-24; Lake B B, Chen S, Sos B C, et al. Integrative single-cell analysis of transcriptional and epigenetic states in the human adult brain. Nat Biotechnol. 2018; 36:70-80; Preissl S, Fang R, Huang H, et al. Single-nucleus analysis of accessible chromatin in developing mouse forebrain reveals cell-type-specific transcriptional regulation. Nat Neurosci. 2018; 21:432-9; Satpathy A T, Granja J M, Yost K E, et al. Massively parallel single-cell chromatin landscapes of human immune cell development and intratumoral T cell exhaustion. Nat Biotechnol. 2019; 37:925-36; Xu W, Wen Y, Liang Y, et al. A plate-based single-cell ATAC-seq workflow for fast and robust profiling of chromatin accessibility. Nat Protoc. 2021; 16:4084-107; US20160208323A1; US20160060691A1; and WO2017156336A1). Single nuclei ATAC-seq can also be performed by partitioning nuclei in droplets and subsequent snATAC-Seq library construction using the Chromium Next GEM Single Cell ATAC Reagent Kit v1.1 (10× Genomics, Pleasanton, CA, USA) (see, e.g., Briel N, Ruf V C, Pratsch K, et al. Single-nucleus chromatin accessibility profiling highlights distinct astrocyte signatures in progressive supranuclear palsy and corticobasal degeneration. Acta Neuropathol. 2022; 144(4):615-635).

In an example embodiment, tagged nuclei are generated according to the present disclosure with spatial barcode nucleic acids comprising an adapter sequence that is the same as the adapters inserted into active chromatin by Tn5 transposase. The tagged chromatin fragments and spatial barcode nucleic acids can then be captured by cell barcodes using a combinatorial indexing or droplet based method using barcoded beads.

### Spatially Resolved Single Cell Hi-C

In example embodiments, the single cell genomics sequencing library is a single cell Hi-C sequencing library. In situ Hi-C involves cross-linking cells with formaldehyde; permeabilizing them with nuclei intact; digesting DNA with a suitable 4-cutter restriction enzyme (such as MboI); filling the 5′-overhangs while incorporating a biotinylated nucleotide; ligating the resulting blunt-end fragments; shearing the DNA; capturing the biotinylated ligation junctions with streptavidin beads; and analyzing the resulting fragments with paired-end sequencing (see, e.g., Rao S S, Huntley M H, Durand N C, et al. A 3D map of the human genome at kilobase resolution reveals principles of chromatin looping. Cell. 2014; 159(7):1665-1680). Hi-C can also be performed in single cells with various barcoding methods, such as combinatorial cellular indexing (see, e.g., Ramani, et al., Sci-Hi-C: A single-cell Hi-C method for mapping 3D genome organization in large number of single cells Methods. 2020 Jan. 1; 170:61-68; Ramani V, Deng X, Qiu R, et al. Massively multiplex single-cell Hi-C. Nat Methods. 2017; 14(3):263-266; and Nagano T, Lubling Y, Várnai C, et al. Cell-cycle dynamics of chromosomal organization at single-cell resolution. Nature. 2017; 547:61-7).

In an example embodiment, tagged nuclei are generated according to the present disclosure with spatial barcode nucleic acids comprising double stranded adapter sequences, tagged nuclei are crosslinked, DNA is digested with a suitable restriction enzyme. Nuclei are then distributed to 96 wells, wherein the first barcode is introduced through ligation of barcoded biotinylated double-stranded bridge adaptors. Intact nuclei are then pooled and subjected to proximity ligation, followed by dilution and redistribution to a second 96-well plate. Following lysis, a second barcode is introduced through ligation of barcoded Y-adaptors. In this example, both the fragmented DNA and the spatial barcodes receive the same nuclei specific barcode.

In an example embodiment, tagged nuclei are generated according to the present disclosure with spatial barcode nucleic acids comprising adapter sequences, tagged nuclei are crosslinked, DNA is digested with a suitable restriction enzyme, the resulting fragments are ligated, the DNA is fragmented with a transposase (Tn5) loaded with adapters in the intact nuclei, the nuclei can then be used in a droplet based single cell sequencing method using beads that capture the adapter sequences and add a cell barcode.

### Spatially Resolved Single Cell DNA-Methylation Sequencing

In example embodiments, the single cell genomics sequencing library is a single cell DNA methylation sequencing library. Methods for distinguishing DNA methylation include (i) bisulfite conversion, (ii) Tet-assisted bisulfite conversion, (iii) Tet-assisted conversion with a substituted borane reducing agent, and (iv) protection of hmC followed by Tet-assisted conversion with a substituted borane reducing agent (see, e.g., US patent Application No. US20210115502A1). Methylation can also be detected using methylation specific restriction enzymes or methylated DNA immunoprecipitation (MeDIP). In example embodiments, DNA methylation can be detected where methylated cytosines (mC) and hydroxymethylated cytosines (hmC) are determined by the sequencer itself and independent of one or more agents (e.g., using PacBio or Nanopore sequencers). Single-cell DNA methylome sequencing techniques have been established using various strategies including the reduced representation bisulfite sequencing (RRBS)- and the post-bisulfite adaptor tagging (PBAT)-based methods (see, e.g., Ahn J, Heo S, Lee J, Bang D. Introduction to Single-Cell DNA Methylation Profiling Methods. Biomolecules. 2021; 11(7):1013; Guo H, Zhu P, Wu X, et al. Single-cell methylome landscapes of mouse embryonic stem cells and early embryos analyzed using reduced representation bisulfite sequencing. Genome Res. 2013; 23:2126-35; Luo C, Keown C L, Kurihara L, et al. Single-cell methylomes identify neuronal subtypes and regulatory elements in mammalian cortex. Science. 2017; 357:600-4; Smallwood S A, Lee H J, Angermueller C, et al. Single-cell genome-wide bisulfite sequencing for assessing epigenetic heterogeneity. Nat Methods. 2014; 11:817-20; and Shareef S J, Bevill S M, Raman A T, et al. Extended-representation bisulfite sequencing of gene regulatory elements in multiplexed samples and single cells. Nat Biotechnol. 2021; 39:1086-94). A sci-MET method has applied a combinatorial indexing strategy for increasing the throughput, with the first and second rounds of barcodes being incorporated by Tn5 transposon and random priming, respectively (Mulqueen R M, Pokholok D, Norberg S J, et al. Highly scalable generation of DNA methylation profiles in single cells. Nat Biotechnol. 2018; 36:428-31).

### Spatially Resolved Single Cell Chromatin Immunoprecipitation (ChIP)

In example embodiments, the single cell genomics sequencing library is a single cell chromatin immunoprecipitation (ChIP) sequencing library. Chromatin immunoprecipitation (ChIP) is a widely used method for detecting modifications of histones in nucleosomes of chromatin. In single-cell ChIP-seq analysis, cell-specific barcodes are added before aggregating the cells for immunoprecipitation (Ai S, Xiong H, Li C C, et al. Profiling chromatin states using single-cell itChIP-seq. Nat Cell Biol. 2019; 21:1164-72; Grosselin K, Durand A, Marsolier J, et al. High-throughput single-cell ChIP-seq identifies heterogeneity of chromatin states in breast cancer. Nat Genet. 2019; 51:1060-6; and Rotem A, Ram O, Shoresh N, et al. Single-cell ChIP-seq reveals cell subpopulations defined by chromatin state. Nat Biotechnol. 2015; 33:1165-72). Among these methods, Drop-ChIP and scChIP-seq add cell barcodes by MNase digestion and ligation with a droplet microfluidics workflow, while itChIP adds cell barcodes by Tn5 transposase tagmentation with a chromatin opening step.

In example embodiments, the spatial barcode is designed to allow tagmentation or digestion and ligation such that a barcode sequence is generated having a cell barcode and spatial barcode. For example, single nuclei can be segregated into individual droplets or microwells containing the cell barcodes. The cell barcode/spatial barcode hybrids can be amplified from the input chromatin used for immunoprecipitation and sequenced. This provides the spatial location of each cell. The immunoprecipitated chromatin can then be assigned to the same cells based on the cell barcode.

### Spatially Resolved Single Cell Enzyme-Tethering Chromatin Profiling

In example embodiments, the single cell genomics sequencing library is a single cell enzyme-tethering chromatin profiling sequencing library. Enzyme-tethering represents a non-immunoprecipitation chromatin profiling approach that is becoming increasingly popular and has been adapted to single-cell analysis (see, e.g., Carter B, Ku W L, Kang J Y, et al. Mapping histone modifications in low cell number and single cells using antibody-guided chromatin tagmentation (ACT-seq). Nat Commun. 2019; 10:3747; Harada A, Maehara K, Handa T, et al. A chromatin integration labelling method enables epigenomic profiling with lower input. Nat Cell Biol. 2019; 21:287-96; Kaya-Okur H S, Wu S J, Codomo C A, et al. CUT&Tag for efficient epigenomic profiling of small samples and single cells. Nat Commun. 2019; 10:1930; Schmid M, Durussel T, Laemmli U K. ChIC and ChEC; genomic mapping of chromatin proteins. Mol Cell. 2004; 16:147-57; Skene P J, Henikoff S. An efficient targeted nuclease strategy for high-resolution mapping of DNA binding sites. Elife. 2017; 6: e21856; and Wang Q, Xiong H, Ai S, et al. CoBATCH for High-Throughput Single-Cell Epigenomic Profiling. Mol Cell. 2019; 76:206-16 . . . e7). In these techniques, Tn5 transposase, MNase, or adenine methyltransferase is tethered to protein A that binds to the antibody, directly to the antibody, or directly to the target chromatin protein, which allows marking of the genomic regions having specific histone marks. ChIC, CUT&RUN, and scChIC use MNase, while scCUT&Tag, COBATCH, ACT-seq, and ChIL-seq use Tn5 transposase. A key cation-activation step, Ca2+ for MNase and Mg2+ for Tn5 transposase, allows activation of the enzyme activity in a short time window after washing off the nonspecifically-bound enzyme.

In an example embodiment, single tagged nuclei according to the present disclosure are tagged with spatial barcode nucleic acids including the same sequence as tagmentation adapters or ligation adapters used in the enzyme-tethering chromatin profiling method. The enzyme-tethering chromatin profiling method adds the adapters to chromatin bound by an antibody specific for a chromatin modification or protein. These nuclei can then be used in a single cell sequencing method that include barcoded beads that comprise sequences that hybridize to the adapter sequences and add a bead specific barcode to the tagged chromatin and spatial barcodes (e.g., 10×ATAC-seq kit). In other words, nuclei are tagged with spatial barcodes, nuclei are isolated, antibodies are added to nuclei, unbound antibodies are washed away, secondary antibodies with Tn5 or MNase are added, Ca²⁺ or Mg²⁺ buffer is added to tag DNA bound by the antibody, and a compatible single cell sequencing method is performed to add cell barcodes to the spatial barcodes and marked chromatin.

### Spatially Resolved Single Cell Proteomics

In example embodiments, the single cell genomics sequencing library comprises a single cell proteomics sequencing library (see, e.g., Yang L, George J, Wang J. Deep Profiling of Cellular Heterogeneity by Emerging Single-Cell Proteomic Technologies. Proteomics. 2020; 20 (13): e1900226. doi: 10.1002/pmic.201900226; single cell constituents (US20180340939A), single-cell proteomic assay using aptamers (US20180320224A1), and methods of identifying multiple epitopes in cells (US20170321251A1)).

In example embodiments, the tagged nuclei are labeled with barcode oligonucleotide linked antibodies or aptamers, the nuclei are washed to remove unbound antibodies or aptamers, and the barcode oligonucleotides of the bound antibodies or aptamers then capture the cell barcode sequence. The oligonucleotide linked antibodies or aptamers include a universal sequence complementary to a capture sequence or handle for pool and split indexing or for capture by cell barcode beads. The spatial barcode nucleic acid is modified to include the same universal sequence, such as to capture the spatial barcode to the cell barcode sequence.

### Spatially Resolved Single Cell Multiomics

In example embodiments, the single cell genomics sequencing library comprises a single cell multiomics sequencing library (see, e.g., Lee J, Hyeon D Y, Hwang D. Single-cell multiomics: technologies and data analysis methods. Exp Mol Med. 2020; 52(9):1428-1442. doi: 10.1038/s12276-020-0420-2; Wen L, Tang F. Single cell epigenome sequencing technologies. Mol Aspects Med. 2018; 59:62-69; Cao J, Cusanovich D A, Ramani V, et al. Joint profiling of chromatin accessibility and gene expression in thousands of single cells. Science. 2018; 361:1380-5; Chen S, Lake B B, Zhang K. High-throughput sequencing of the transcriptome and chromatin accessibility in the same cell. Nat Biotechnol. 2019; 37:1452-7; Liu L, Liu C, Quintero A, et al. Deconvolution of single-cell multi-omics layers reveals regulatory heterogeneity. Nat Commun. 2019; 10:470; Zhu C, Yu M, Huang H, et al. An ultra high-throughput method for single-cell joint analysis of open chromatin and transcriptome. Nat Struct Mol Biol. 2019; 26:1063-70; Ma, S. et al. Chromatin potential identified by shared single cell profiling of RNA and chromatin. bioRxiv 2020.06.17.156943 (2020) doi: 10.1101/2020.06.17.156943; and Stoeckius, M. et al. Simultaneous epitope and transcriptome measurement in single cells. Nat. Methods 14, 865-868 (2017). In example embodiments, for multiomic assays, the spatial barcode needs to be captured by a cell barcode for only one of the omic parts of the multiomic single cell assay. For example, the spatial barcode can be configured to be captured by a poly-T sequence if the multiomic assay includes transcriptome sequencing.

### Imaging/Image Assembly

With spatial barcodes of individual nuclei identified by cell of origin barcodes, and with analytes identified by sequences also including the cell of origin barcodes also identified, high-resolution images that localize sites of analyte expression can be readily constructed in silico. In example embodiments, the spatial locations of a large number of sites (e.g., beads) within an array can first be assigned to an image location, with all associated analyte expression data also assigned to that position (optionally, effectively de-coupling the spatial barcode from the array/matrix of analyte sequence information associated with a given site/bead, once the spatial barcode has been used to assign the analyte sequence information to an array position). High resolution images representing the extent of individual or grouped analytes across the various spatial positions of the arrays can then be generated using the underlying analyte sequence information. Images (i.e., pixel coloring and/or intensities) can be adjusted and/or normalized using any (or any number of) art-recognized technique(s) deemed appropriate by one of ordinary skill in the art.

In example embodiments, a high-resolution image of the instant disclosure is an image in which discrete features (e.g., pixels) of the image are spaced at 50 μm or less. In some embodiments, the spacing of discrete features within the image is at 40 μm or less, optionally 30 μm or less, optionally 20 μm or less, optionally 15 μm or less, optionally 10 μm or less, optionally 9 μm or less, optionally 8 μm or less, optionally 7 μm or less, optionally 6 μm or less, optionally 5 μm or less, optionally 4 μm or less, optionally 3 μm or less, optionally 2 μm or less, or optionally 1 μm or less.

Images can be obtained using detection devices known in the art. Examples include microscopes configured for light, bright field, dark field, phase contrast, fluorescence, reflection, interference, or confocal imaging. A biological specimen can be stained prior to imaging to provide contrast between different regions or cells. In some embodiments, more than one stain can be used to image different aspects of the specimen (e.g. different regions of a tissue, different cells, specific subcellular components or the like). In other embodiments, a biological specimen can be imaged without staining.

In particular embodiments, a fluorescence microscope (e.g., a confocal fluorescent microscope) can be used to detect a biological specimen that is fluorescent, for example, by virtue of a fluorescent label. Fluorescent specimens can also be imaged using a nucleic acid sequencing device having optics for fluorescent detection such as a Genome Analyzer®, MiSeq®, NextSeq® or HiSeq® platform device commercialized by Illumina, Inc. (San Diego, Calif.); or a SOLID™ sequencing platform commercialized by Life Technologies (Carlsbad, Calif.). Other imaging optics that can be used include those that are found in the detection devices described in Bentley et al., Nature 456:53-59 (2008), PCT Publ. Nos. WO 91/06678, WO 04/018497 or WO 07/123744; U.S. Pat. Nos. 7,057,026, 7,329,492, 7,211,414, 7,315,019 or 7,405,281, and US Pat. App. Publ. No. 2008/0108082, each of which is incorporated herein by reference.

An image of a biological specimen can be obtained at a desired resolution, for example, to distinguish tissues, cells or subcellular components. Accordingly, the resolution can be sufficient to distinguish components of a biological specimen that are separated by at least 0.5 μm, 1 μm, 5 μm, 10 μm, 50 μm, 100 μm, 500 μm, 1 mm or more. Alternatively or additionally, the resolution can be set to distinguish components of a biological specimen that are separated by at least 1 mm, 500 μm, 100 μm, 50 μm, 10 μm, 5 μm, 1 μm, 0.5 μm or less.

A method set forth herein can include a step of correlating locations in an image of a biological specimen with barcode sequences of nucleic acid probes that are attached to individual beads to which the biological specimen is, was or will be contacted. Accordingly, characteristics of the biological specimen that are identifiable in the image can be correlated with the nucleic acids that are found to be present in their proximity. Any of a variety of morphological characteristics can be used in such a correlation, including for example, cell shape, cell size, tissue shape, staining patterns, presence of particular proteins (e.g., as detected by immunohistochemical stains) or other characteristics that are routinely evaluated in pathology or research applications. Accordingly, the biological state of a tissue or its components as determined by visual observation can be correlated with molecular biological characteristics as determined by spatially resolved nucleic acid analysis.

A solid support upon which a biological specimen is imaged can include fiducial markers to facilitate determination of the orientation of the specimen or the image thereof in relation to probes that are attached to the solid support. Exemplary fiducials include, but are not limited, to beads (with or without fluorescent moieties or moieties such as nucleic acids to which labeled probes can be bound), fluorescent molecules attached at known or determinable features, or structures that combine morphological shapes with fluorescent moieties. Exemplary fiducials are set forth in US Pat. App. Publ. No. 2002/0150909 A1 or U.S. patent application Ser. No. 14/530,299, each of which is incorporated herein by reference. One or more fiducials are preferably visible while obtaining an image of a biological specimen. Preferably, the solid support includes at least 2, 3, 4, 5, 10, 25, 50, 100 or more fiducial markers. The fiducials can be provided in a pattern, for example, along an outer edge of a solid support or perimeter of a location where a biological specimen resides. In one embodiment, one or more fiducials are detected using the same imaging conditions used to visualize a biological specimen. However, if desired separate images can be obtained (e.g., one image of the biological specimen and another image of the fiducials) and the images can be aligned to each other.

In example embodiments, spatial filtering can be used to clean up data. In one example, specific beads that are lost are filtered out. Total spatial barcode nUMIs can be used to remove beads.

In example embodiments of Slide-tags, spatial barcode oligonucleotides are released into tissue sections to ‘tag’ nuclei. Nuclei are then isolated from the tissue and the spatial barcodes are sequenced, alongside other modalities of molecular information. Importantly, each nucleus receives many spatial barcode oligonucleotides from different locations on the bead arrays. Therefore, nuclei must be computationally positioned in space from the distribution of spatial barcodes each nucleus receives. In example embodiments, density-based spatial clustering of applications with noise (DBSCAN) is used to localize nuclei to their spatial positions (see, e.g., Ester, M., Kriegel, H. -P., Sander, J. & Xu, X. A density-based algorithm for discovering clusters in large spatial databases with noise. In Proc. Second International Conference on Knowledge Discovery and Data Mining (eds Simoudis, E. et al.) 226-231 (AAAI Press, 1996); and Hahsler, M., Piekenbrock, M. & Doran, D. dbscan: fast density-based clustering with R. J. Stat. Softw. 91, 1-30 (2019)). DBSCAN is applied to distinguish “signal” spatial barcodes (those likely to provide value in positioning nuclei) from background “noise” spatial barcodes (likely to confound nuclei positioning). DBSCAN outputs a cluster assignment for each spatial barcode. Cluster=0 denotes noise barcodes, and cluster >0 denotes signal barcodes grouped with other signal barcodes that cluster in space. Spatial positions with all spatial barcodes denoted noise are not assigned to nuclei, or assigned to nuclei with multiple signal clusters. From the remaining nuclei with one distinct spatial barcode signal cluster, a weighted centroid of spatial barcode coordinates in the signal cluster is taken, where weights are the number of unique molecular identifiers (UMIs) for sequenced barcodes. Importantly, DBSCAN requires two parameters: minPts and eps (effectively, radius). To determine the optimal parameter set for each Slide-tags run, we 15 different minPts parameters are iterated through, and the parameter set with the highest proportion of nuclei that are assigned a spatial position is chosen (one DBSCAN signal cluster).

While DBSCAN positions nuclei with relatively high sensitivity and specificity, it is not the only approach for determining positions from a set of spatial barcode coordinates. Example alternatives include assigning a nucleus the position of its highest UMI spatial barcode or taking a weighted 2-dimensional median of spatial barcode coordinates. Other methods to distinguish signal barcodes from noise barcodes include (1) K-means clustering, (2) Affinity propagation, (3) Mean Shift, (4) Spectral Clustering, (5) Agglomerative Clustering, (6) DBSCAN extensions such as HDBSCAN and OPTICS.

In example embodiments, the position of nuclei in the Z-plane may be measured by using individually, or in combination: the counts of spatial barcode UMIs per nucleus, the spread of spatial barcode coordinates per nucleus (e.g., median pairwise distance or similar), fluorescence measurements fluorescently labelled spatial barcodes retained in the nuclei.

### Kits

The instant disclosure also provides kits containing agents of this disclosure for use in the methods of the present disclosure. Kits of the instant disclosure may include one or more containers comprising an agent (e.g., a capture material, such as liquid electrical tape) and/or composition (e.g., a slide-captured bead array) of this disclosure. In example embodiments, the kit includes beads comprising spatial barcode nucleic acids as described herein. In example embodiments, the beads are configured for a specific single cell genomics assay as described herein. In some embodiments, the kits further include instructions for use in accordance with the methods of this disclosure. In some embodiments, the instructions comprise a description of how to create a tissue cryosection, form a spatially-defined (or simply spatially definable, pending performance of a step that defines the spatial resolution of the bead array) bead array, contact a tissue cryosection with a spatially-defined bead array, and how to use the tagged nuclei for subsequent single cell assays.

Instructions supplied in the kits of the instant disclosure are typically written instructions on a label or package insert (e.g., a paper sheet included in the kit), but machine-readable instructions (e.g., instructions carried on a storage drive or provided on the internet) are also acceptable. The label or package insert indicates that the composition is used for staging a cryosection and/or diagnosing a analyte pattern in a cryosection. Instructions may be provided for practicing any of the methods described herein.

The kits of this disclosure are in suitable packaging. Suitable packaging includes, but is not limited to, vials, bottles, jars, flexible packaging (e.g., sealed Mylar or plastic bags), and the like. The container may further comprise a pharmaceutically active agent.

Kits may optionally provide additional components such as buffers and interpretive information. Normally, the kit comprises a container and a label or package insert(s) on or associated with the container.

Further embodiments are illustrated in the following Examples which are given for illustrative purposes only and are not intended to limit the scope of the invention.

### EXAMPLES

### Example 1—Slide-Tag Protocol

### Prepare:

A stock of 500 mLs Dissociation Buffer (DB) using ultrapure nuclease-free water and these reagents:
Na2so4—5.83 g
K2so4—2.615 g
Glucose—0.905 g
HEPES—1.2 g
Mgcl2—2.5 mL

### Extraction Buffer: ExB

DB-15 mL
1% Kollidon—0.150 g
1% TX-100—150 ul
10% BSA—15 ul
1 bottle of RNase inhibitor (lucigen)

### Wash Buffer: WB (20 ml Per Sample)

DB—20 mL
10% BSA—20 ul
50 ul of RNase inhibitor

### Steps:

1. Make a 3 mm 20 um tissue section.
   Note: With the paintbrush, gently press the sectioned tissue to make it flat.
2. Transfer this tissue onto a 3 mm puck and melt the tissue with your finger.
   Note: Place the puck on the glass slide. Keep this glass slide on ice until you transfer the tissue. Right before you transfer tissue on the puck place the glass slide in the cryostat. Transfer the sectioned tissue to the puck, center the tissue on the puck with a brush and tap on the tissue very gently and melt the tissue with a finger. This step must be done as quickly as possible otherwise tissue starts folding.
3. Put 5 ul of DB on the puck, photocleave for 3 minutes and then incubate it for 5 minutes on ice.
4. Transfer the puck to the well plate.
   Note: Holding at the edge with pointed forceps transfer the puck.
5. With a 200/1000 ul pipette dissociate the tissue on the puck using extraction buffer.
   Note: with 200/1000 ul pipette put extraction buffer on the puck. The total volume of extraction buffer used is 2 ml. The pressure of the pipetting should be medium. Take a look in the microscope every 15-20 rounds of pipetting to see if tissue is dissociated. Repeat this until the tissue is dissociated.
6. After the tissue is dissociated, take the puck out of the well.
7. Transfer the dissociated sample into 20 ml wash buffer; divide this into two 50 ml tubes.
8. Centrifuge at 600 g for 10 minutes at 4 C.
9. Take out the supernatant and leave 500 ul pellets in each tube; pool the pellets to make 1 ml.
10. Filter the solution using a 40-micron filter.
11. Add 1:1000 DAPI to the filtered solution and incubate it for 8-10 minutes.
12. Centrifuge at 200 g for 10 minutes at 4 C.
13. Take out the supernatant leaving a 50 ul pellet.
14. Count the nuclei using a hemocytometer and load 43.3 ul into 10×.

### Example 2—Slide-Tags Description and Results

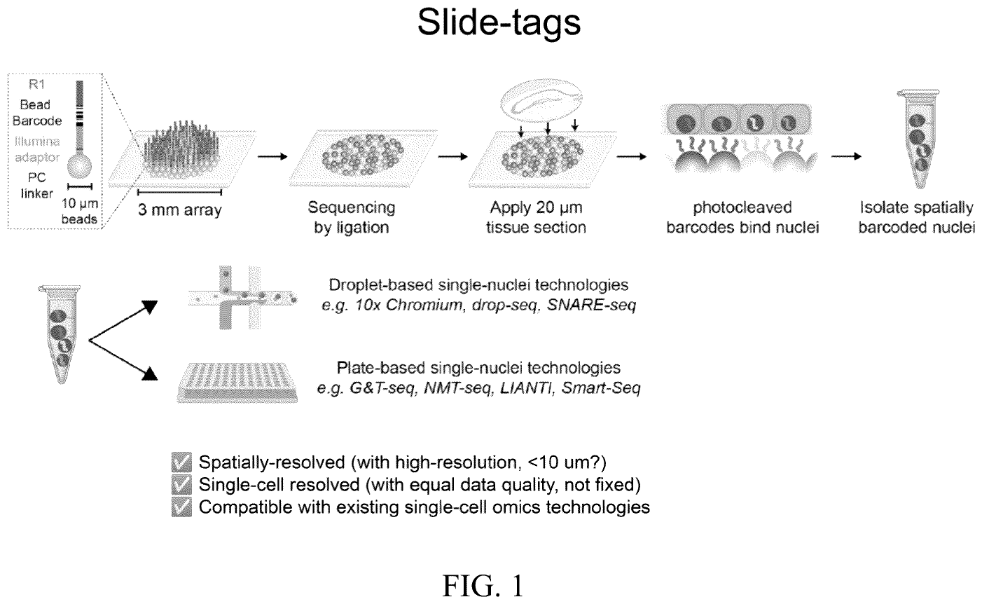

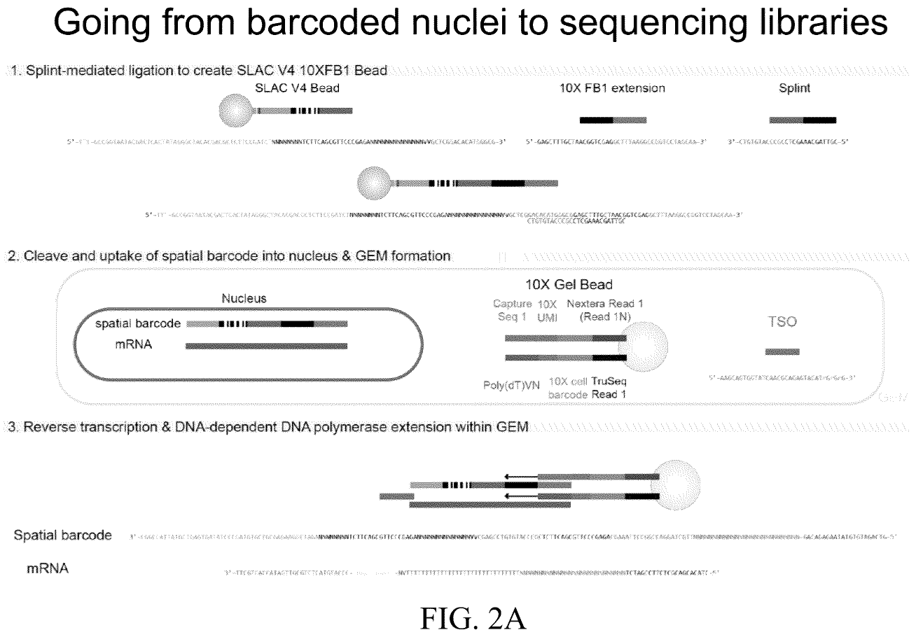

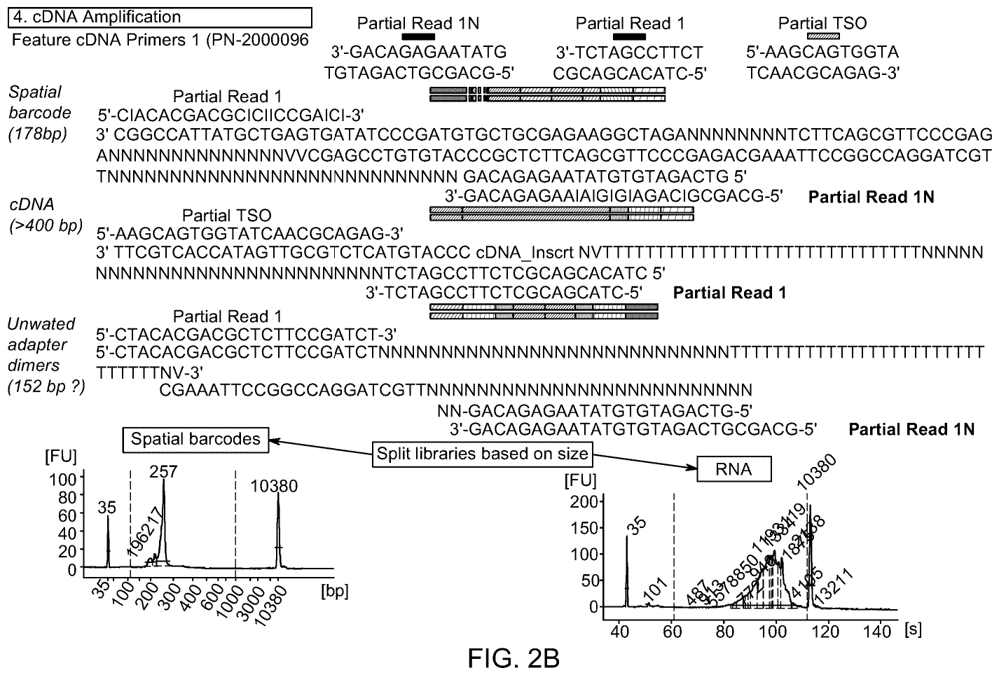

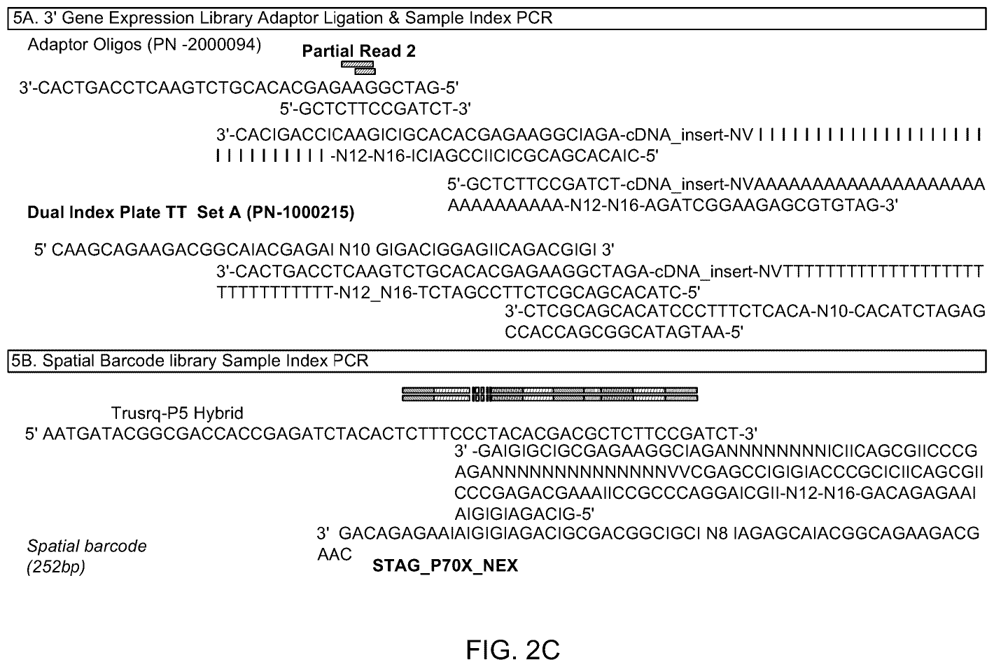

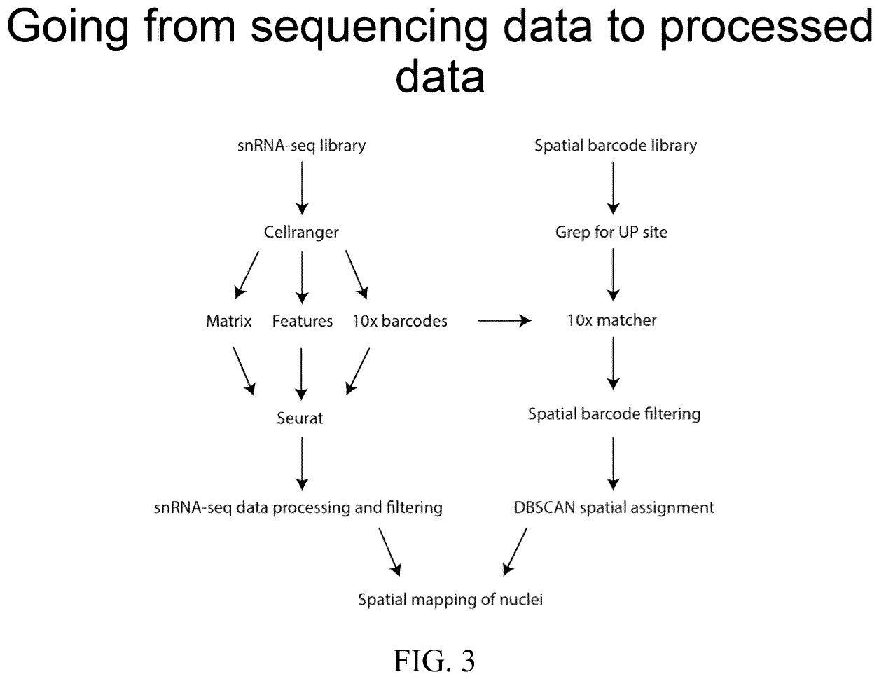

The slide tag method is described in FIGS. 1, 2, and 3. In the first step of slide-tags a thin section of fresh frozen tissue is placed onto barcoded bead arrays, where each bead contains tens of thousands of oligos with the same spatial barcode, and the sequence and position of each spatial-bead-barcode is known. These oligos are photocleaved to release them into the tissue, where they “tag” nuclei. These tagged nuclei are subsequently isolated. Finally, these nuclei can serve as input to established single cell sequencing technologies. This offers the user, true single-cell resolution which is indistinguishable in data quality from a snRNA-seq-only experiment, whilst also providing spatial coordinates for each nucleus. The protocol is easy to implement with the tagging process only adding an extra 10 or so minutes to the workflow.

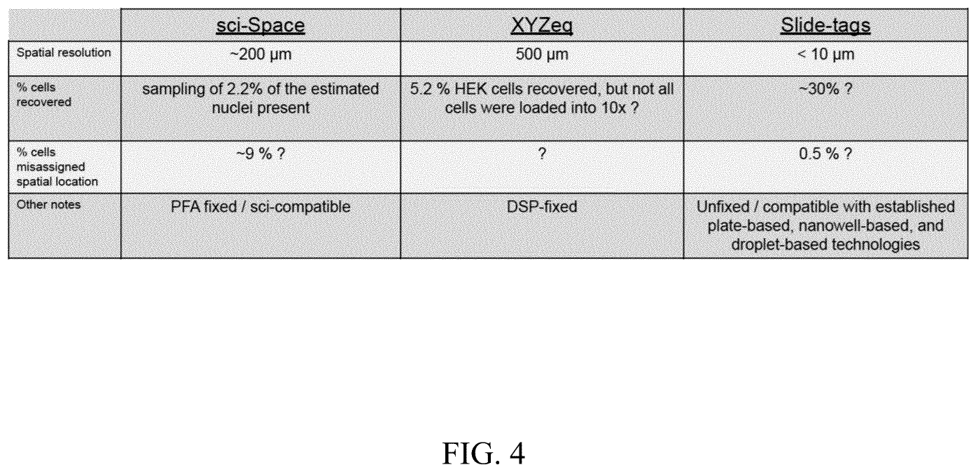

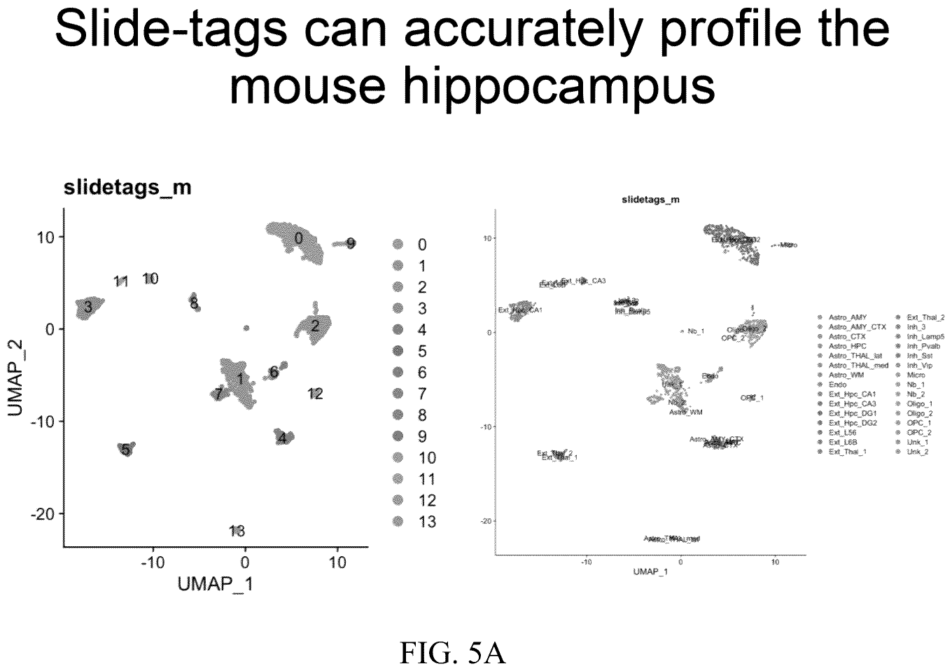

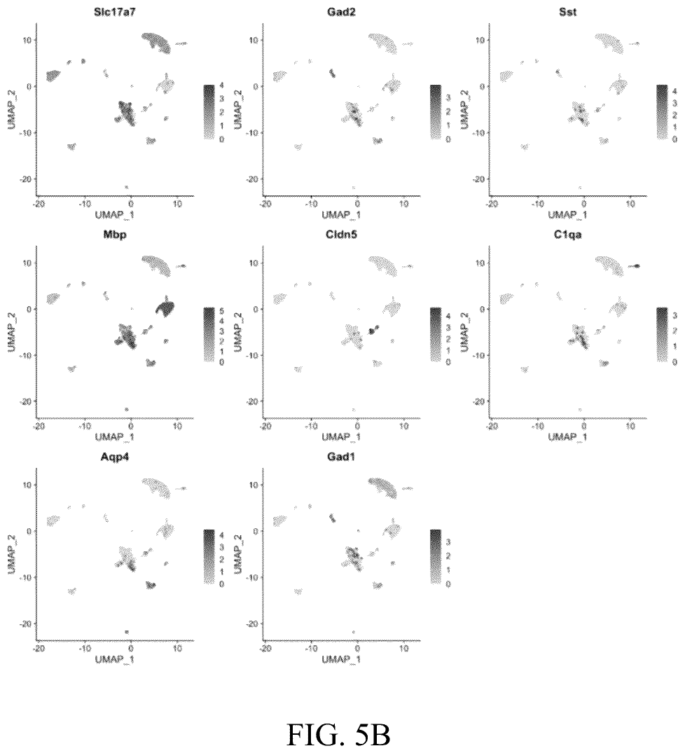

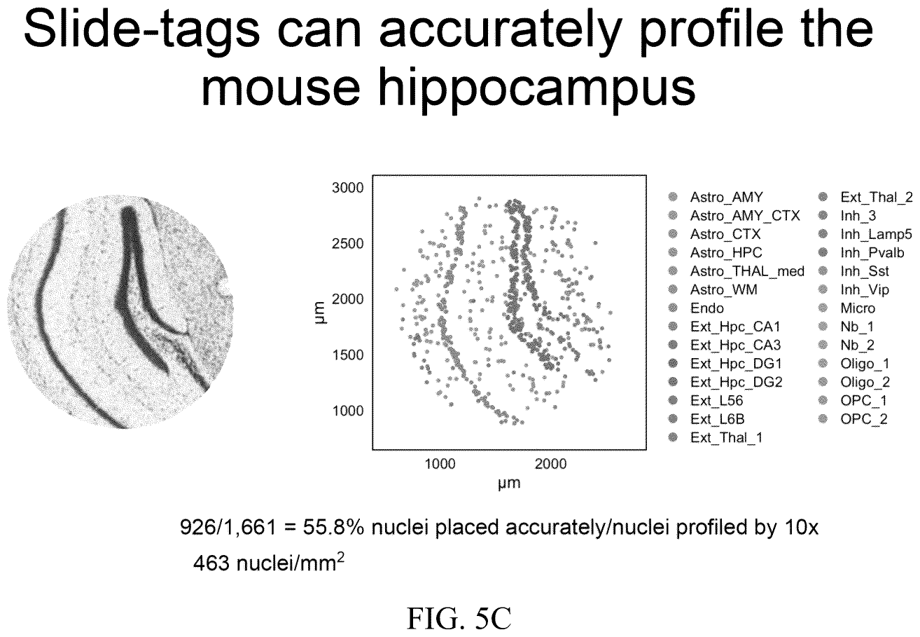

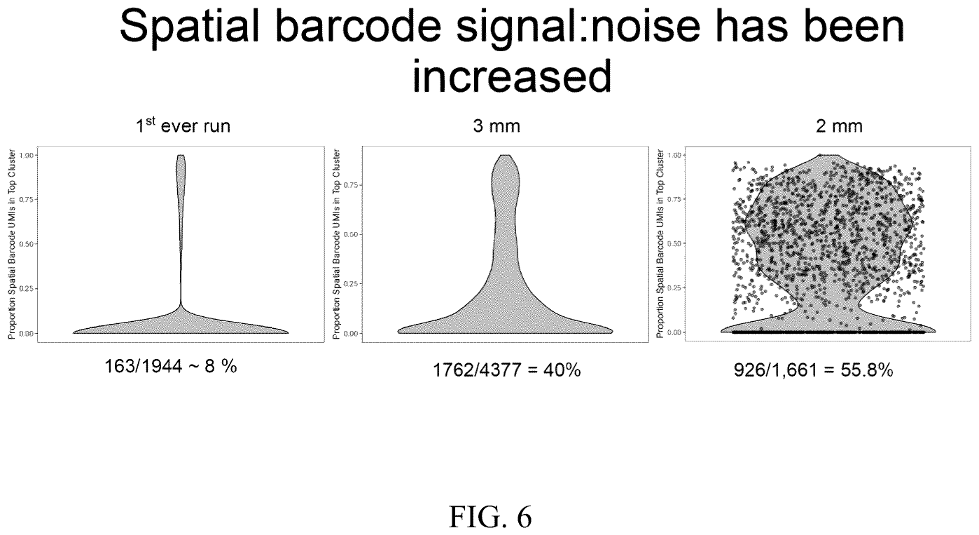

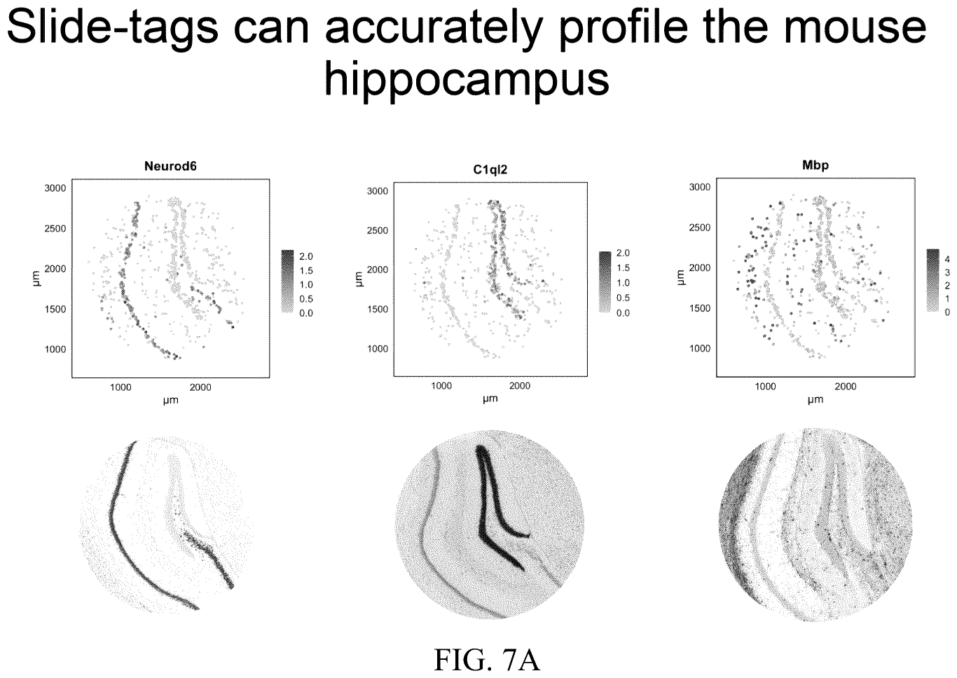

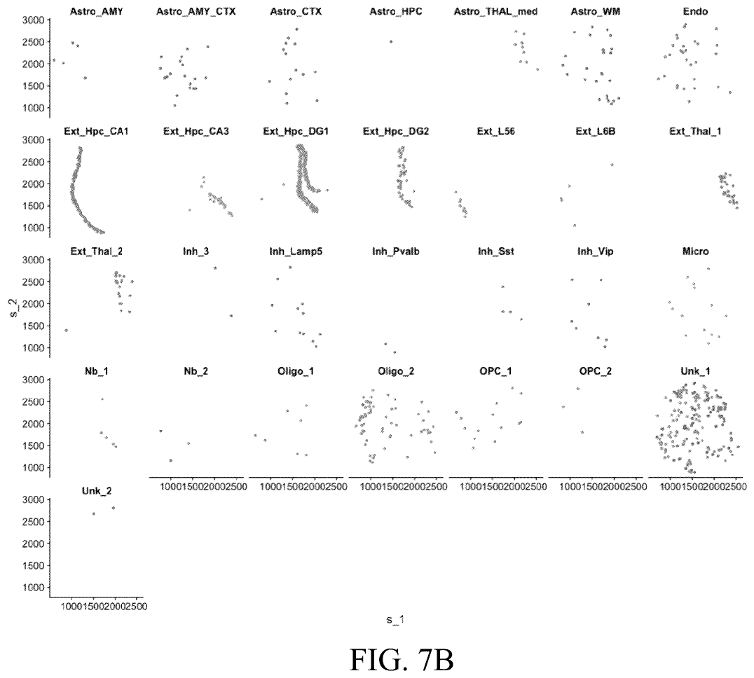

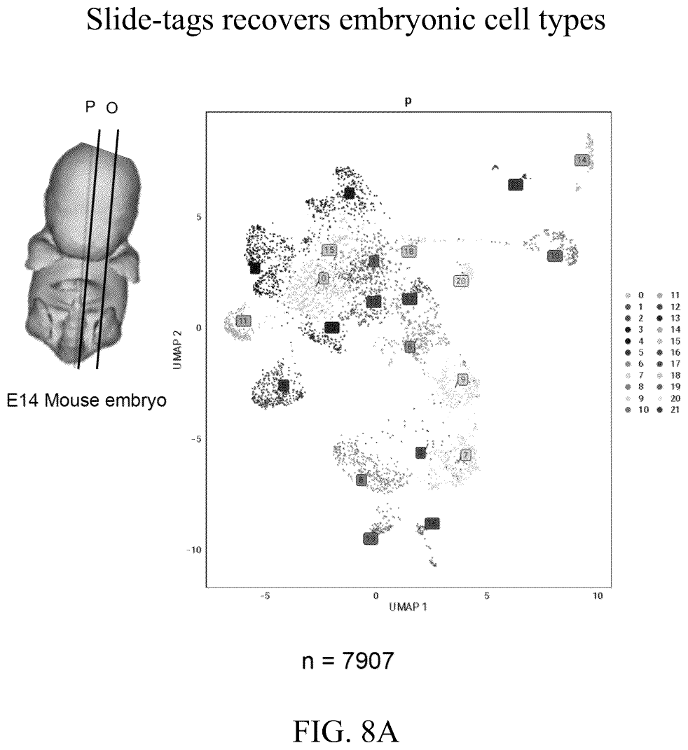

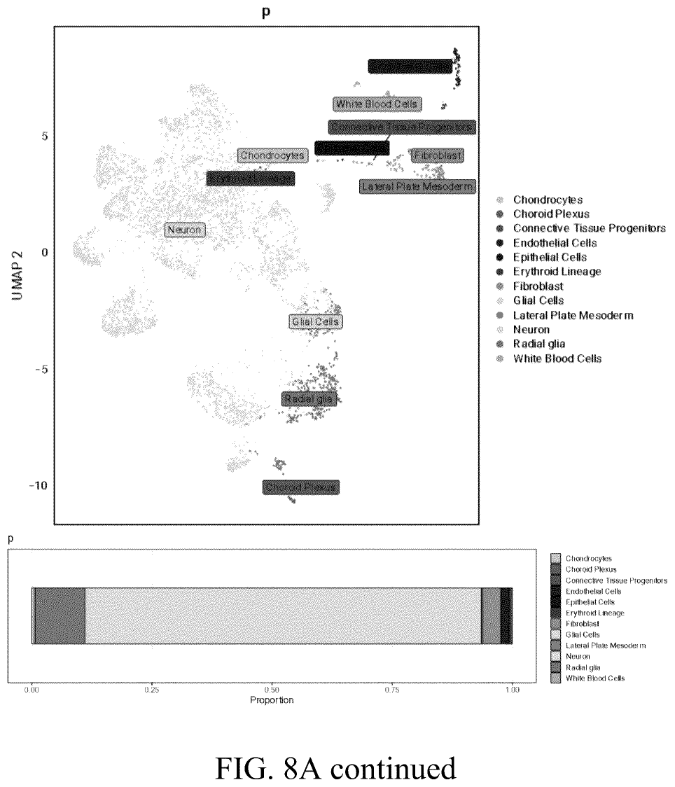

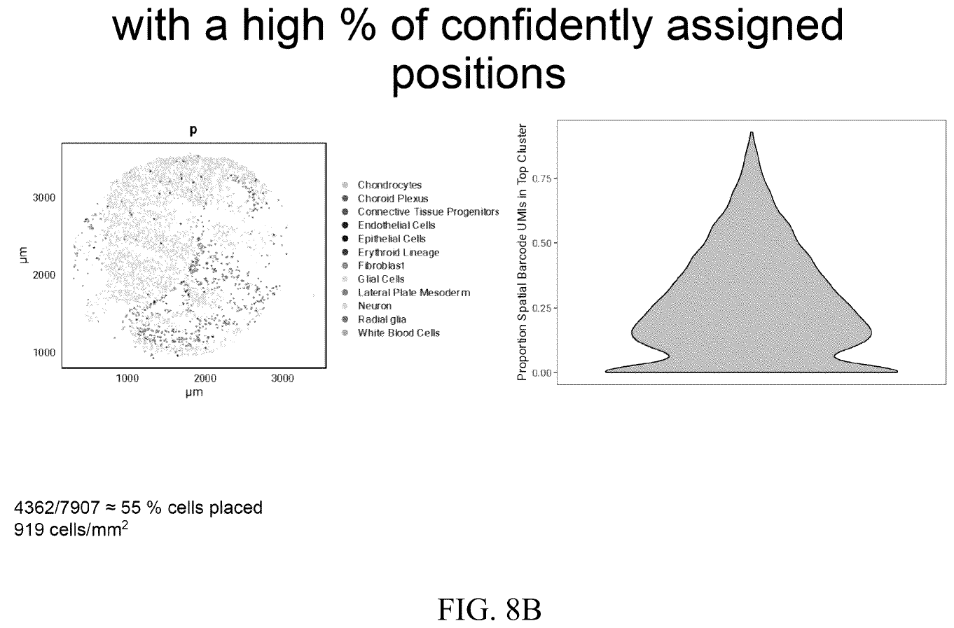

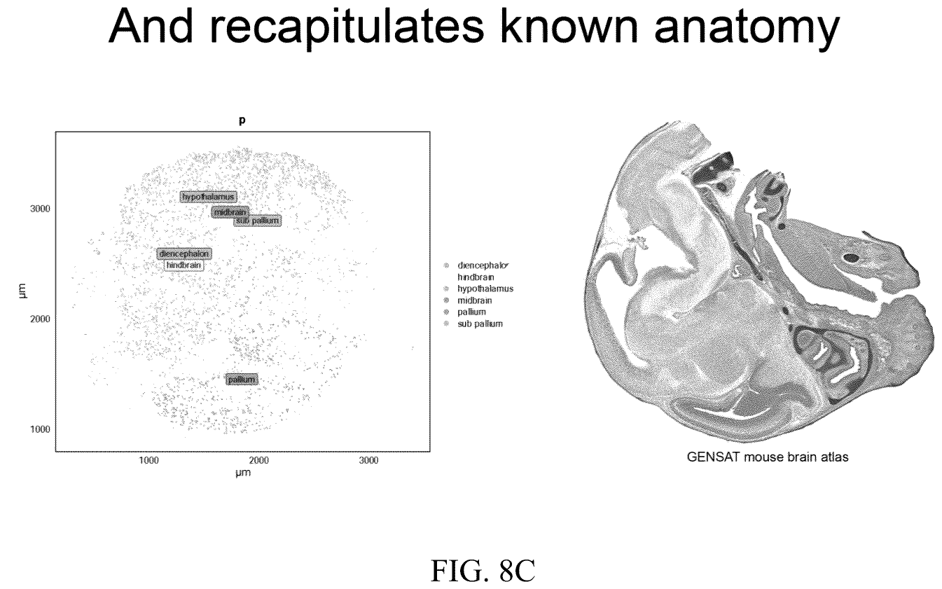

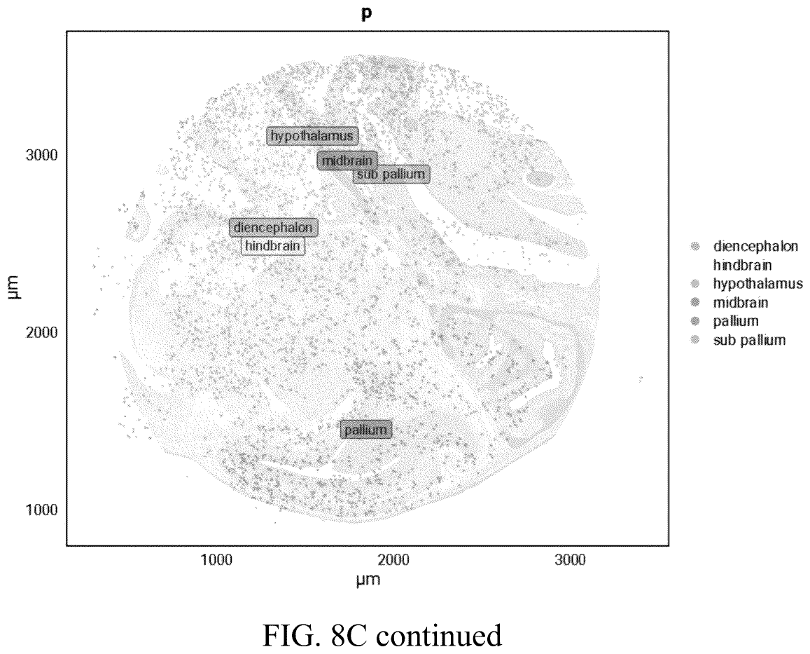

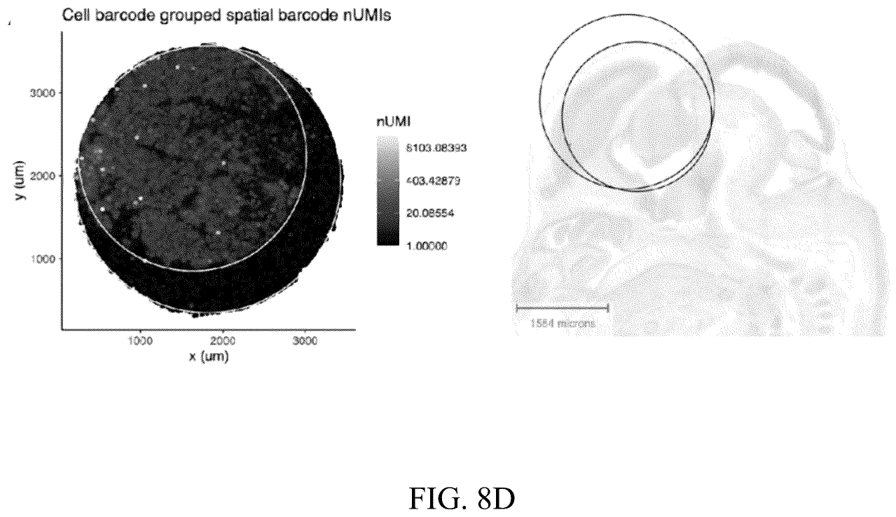

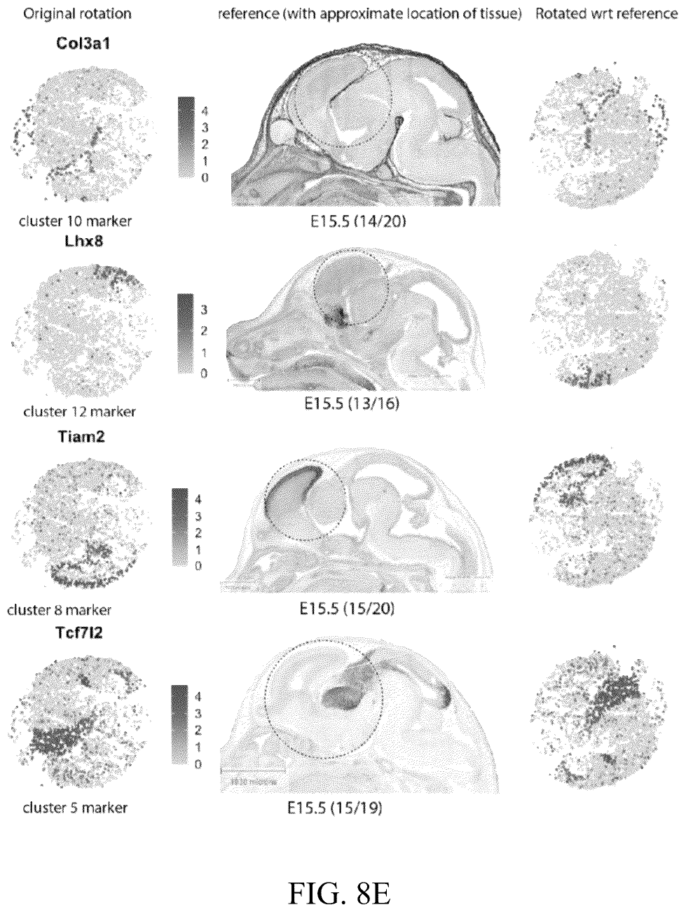

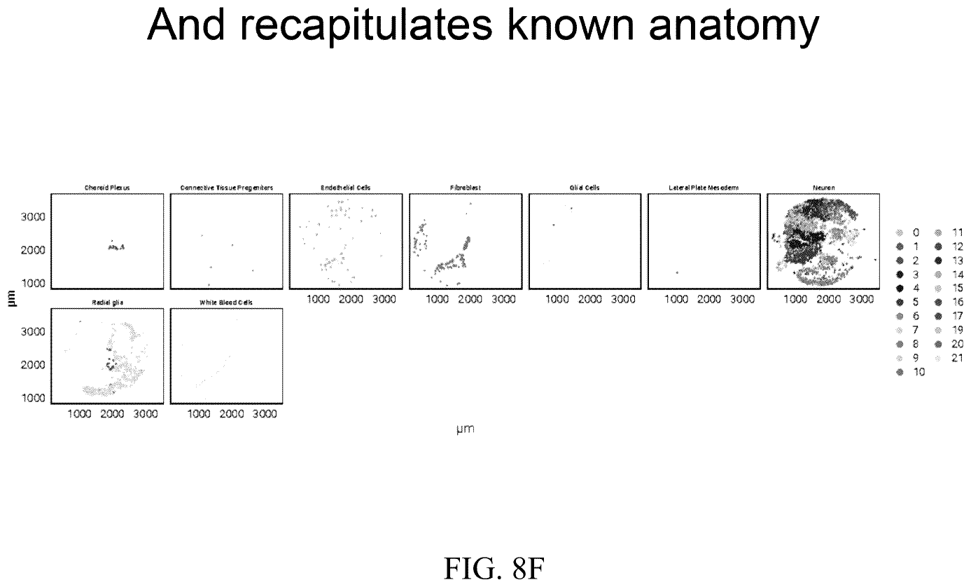

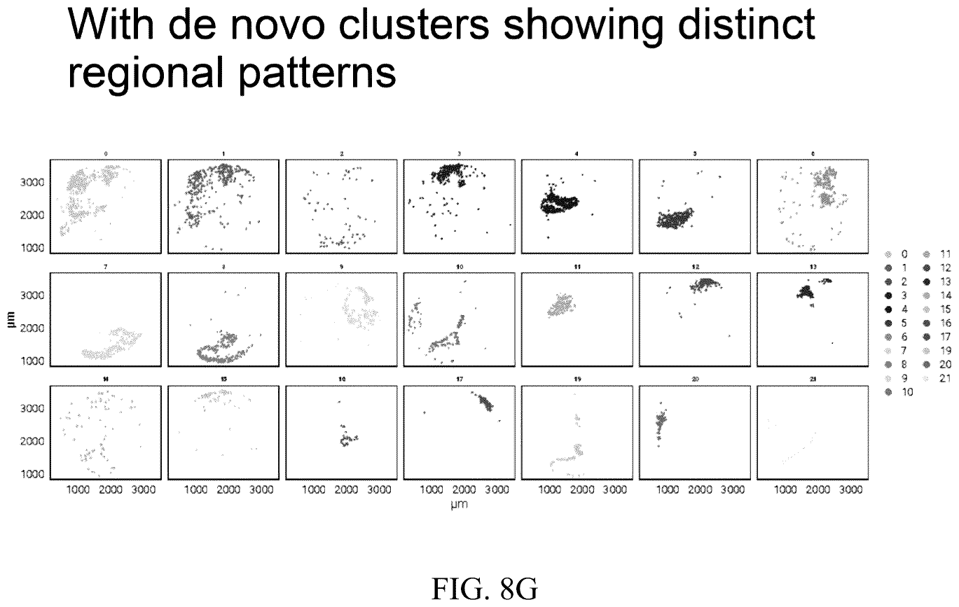

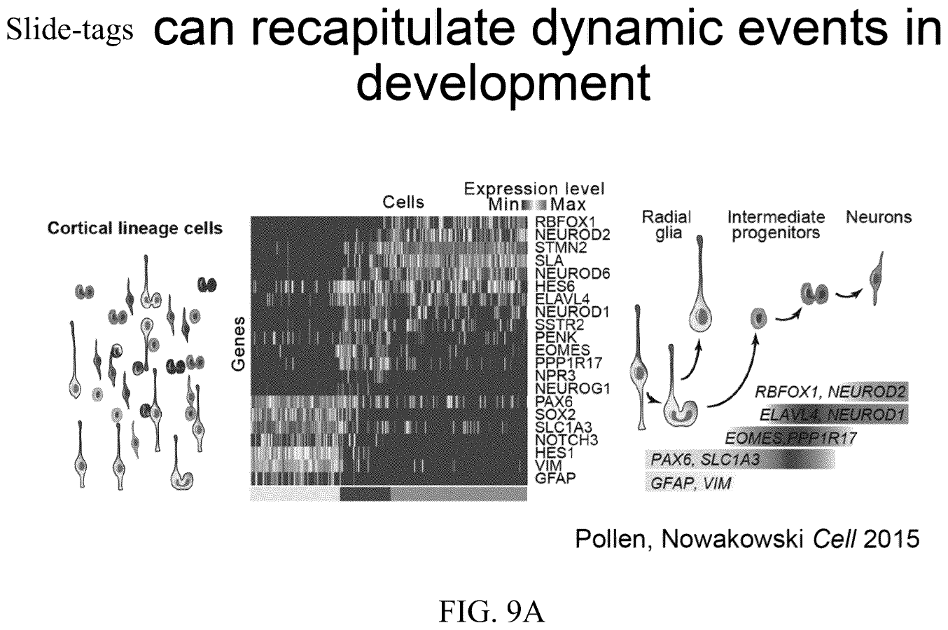

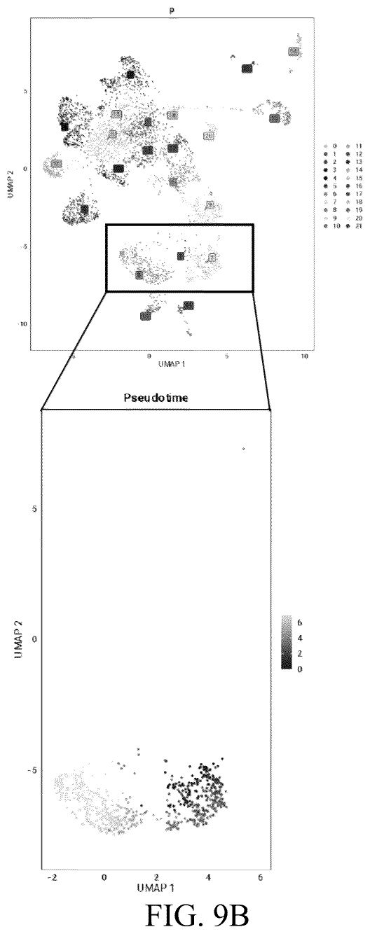

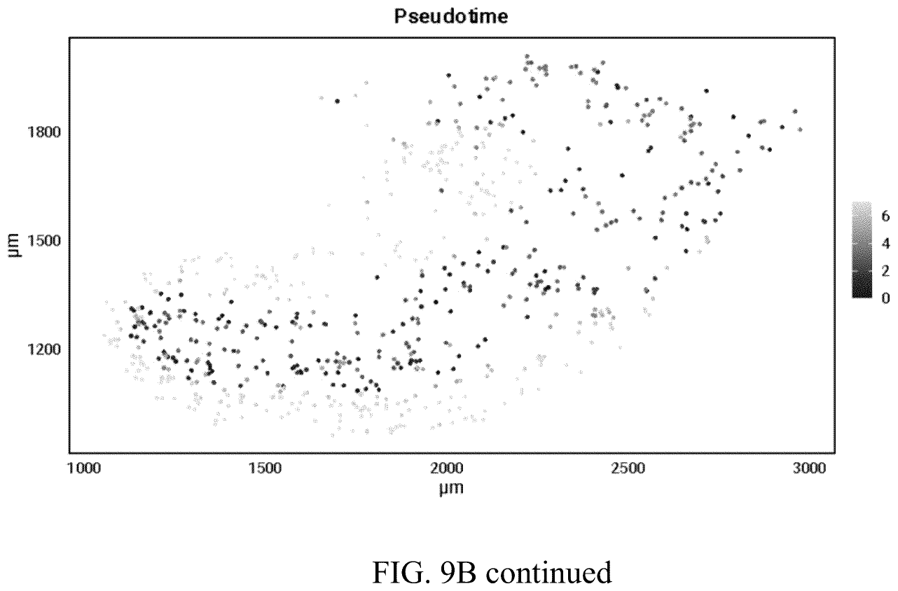

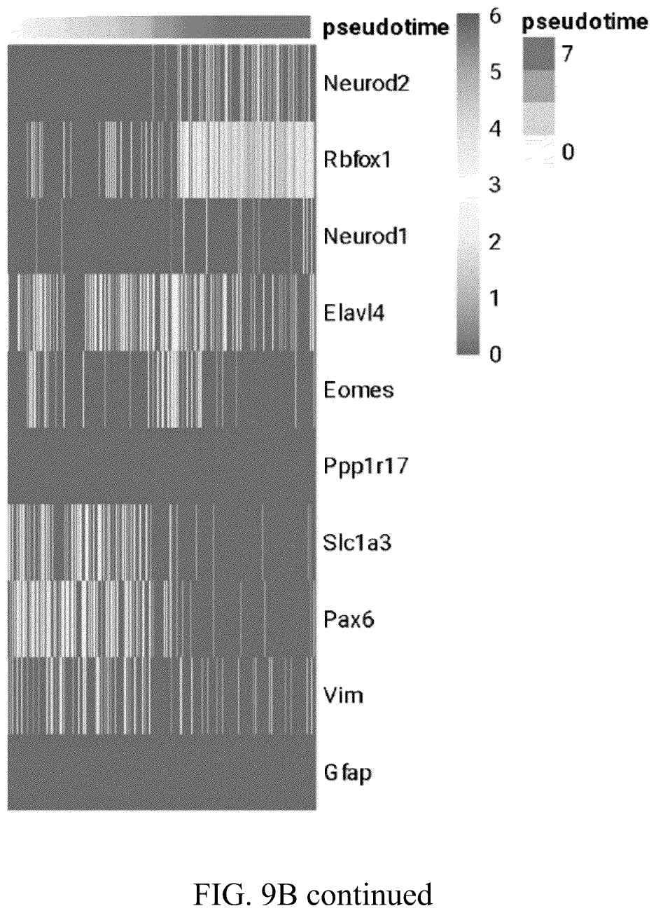

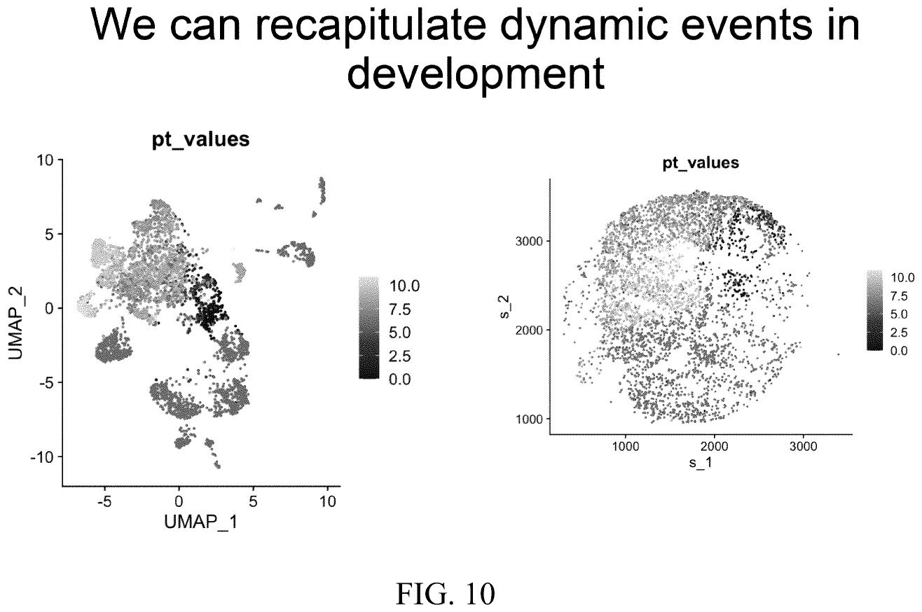

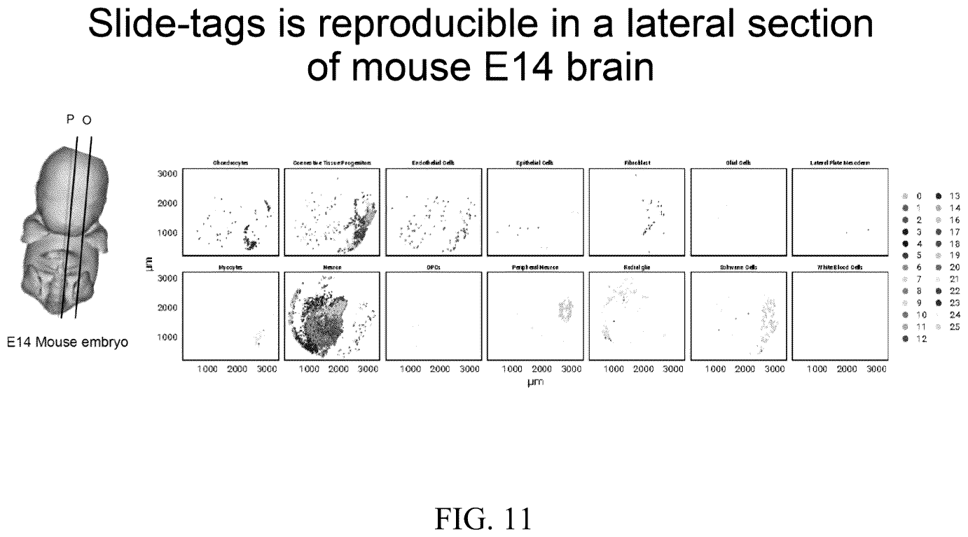

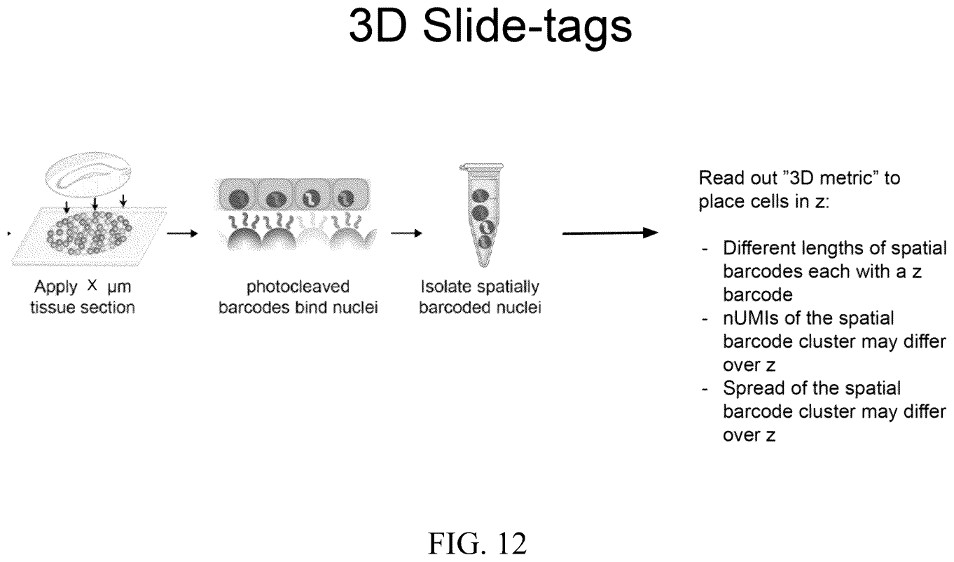

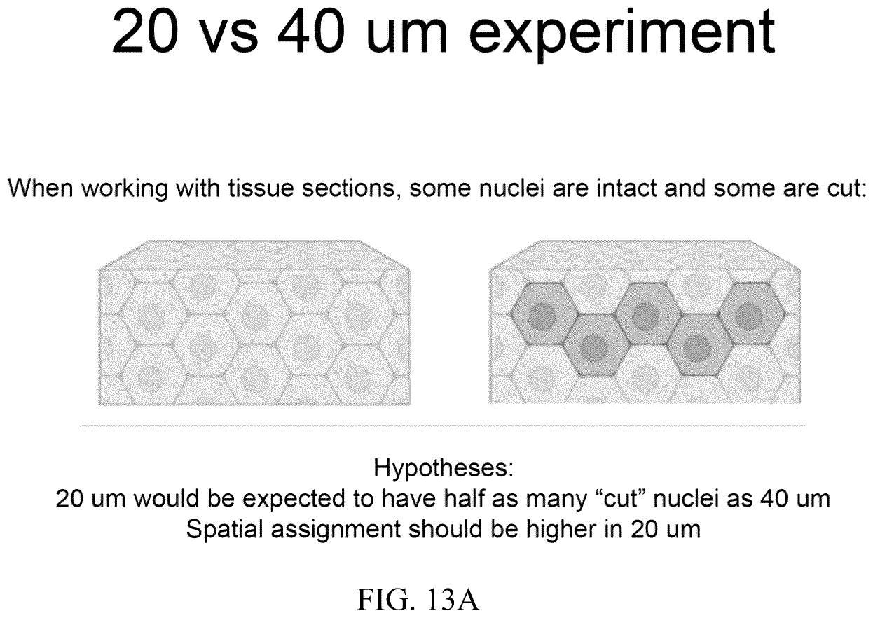

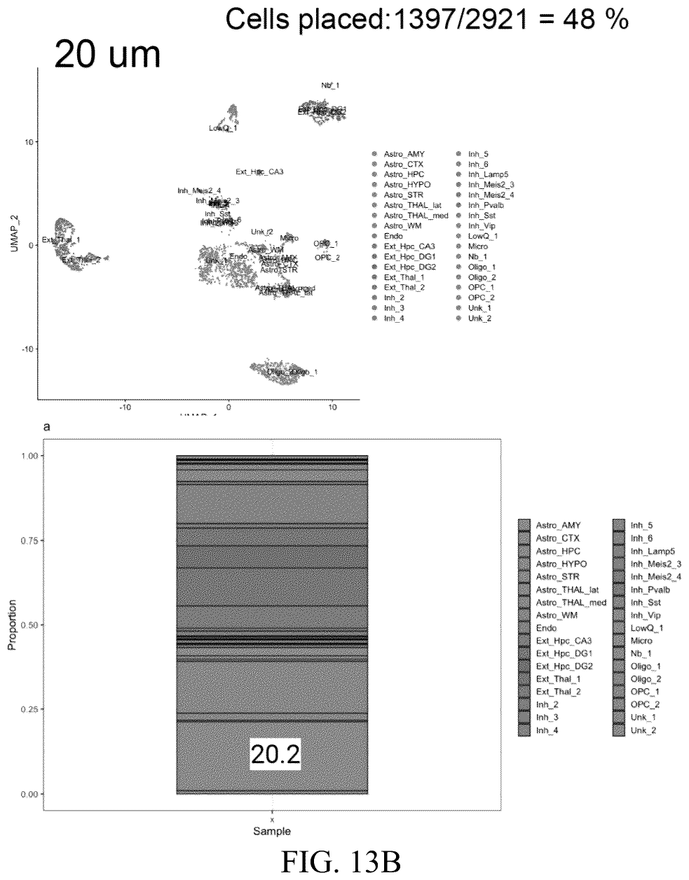

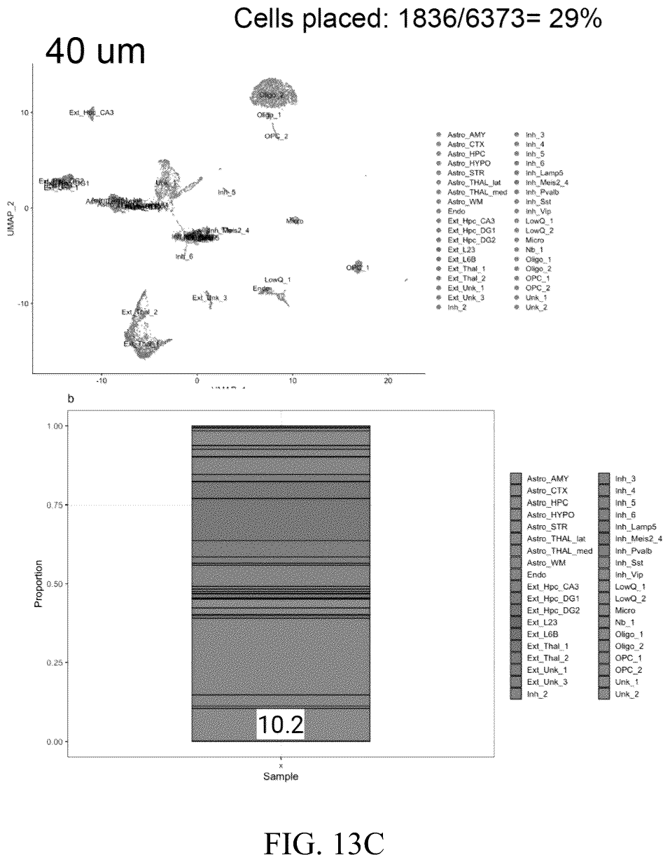

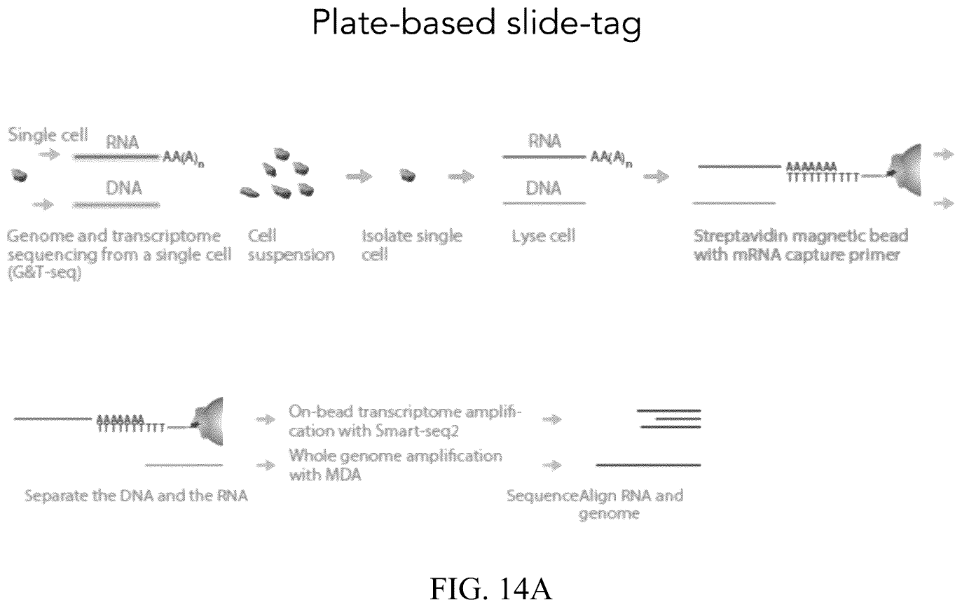

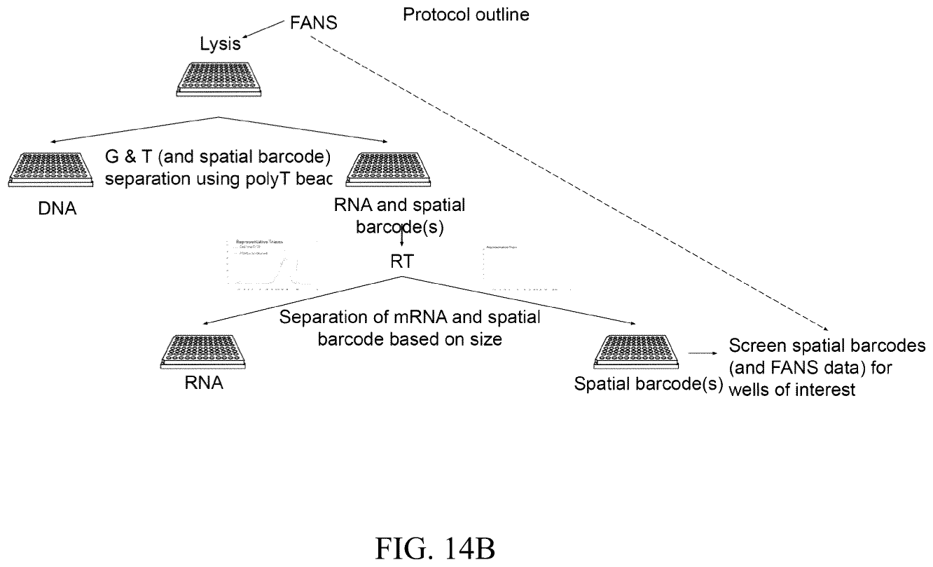

Slide-tags has many advantages over other true spatial methods (FIG. 4). Slide-tags has been optimized to increase the percentage of cells that can be spatially located (FIG. 6). Slide-tags can accurately profile the mouse hippocampus (FIG. 5, FIG. 7). Slide-tags recovers embryonic cell types with a high % of confidently assigned positions and recapitulates known anatomy with de novo clusters showing distinct regional patterns (FIG. 8, FIG. 11). Slide-tags can recapitulate dynamic events in development (FIG. 9, FIG. 10). Slide-tags can provide 3D spatial data (FIG. 12). Spatial assignment is higher in thinner tissue sections (FIG. 13). Slide-tags can be used for spatially resolved single cell multiomic experiments (FIG. 14).

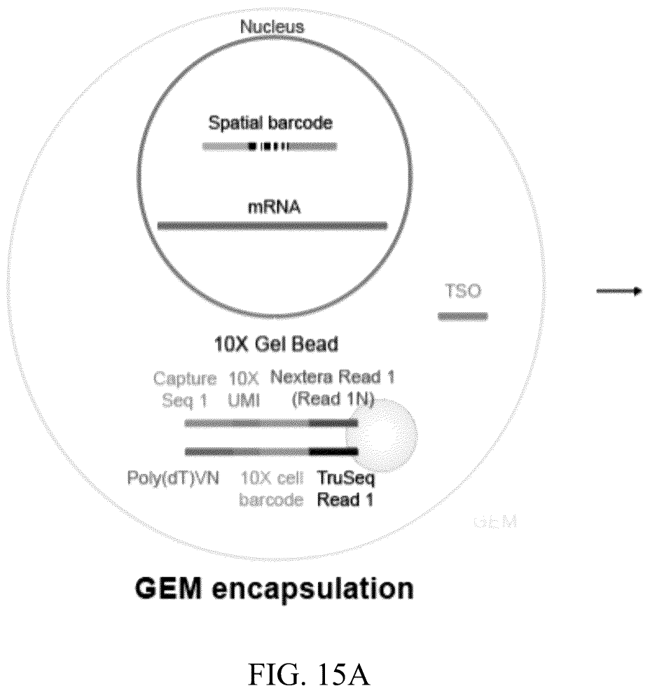

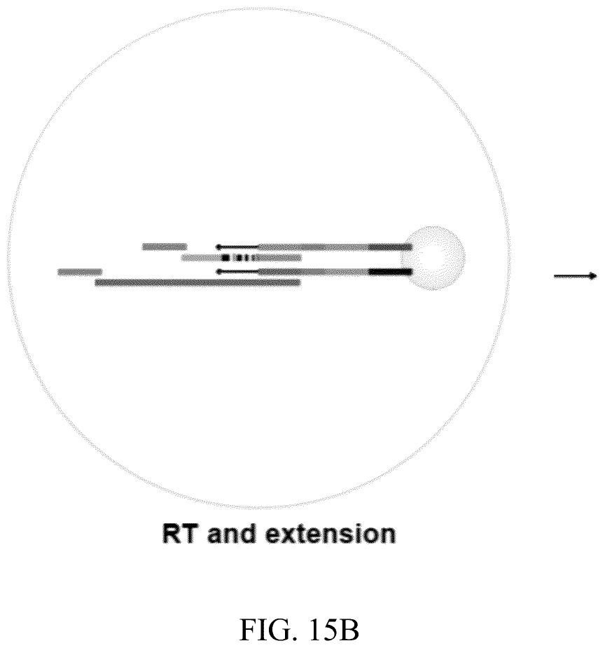

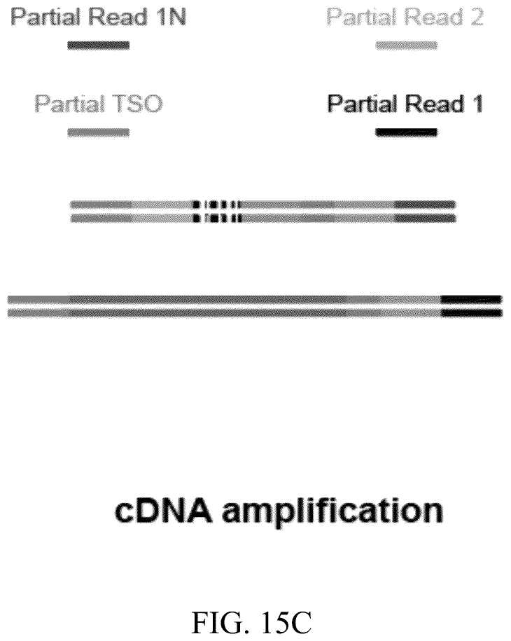

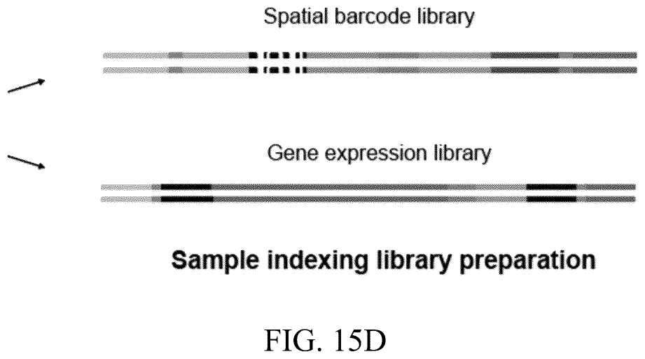

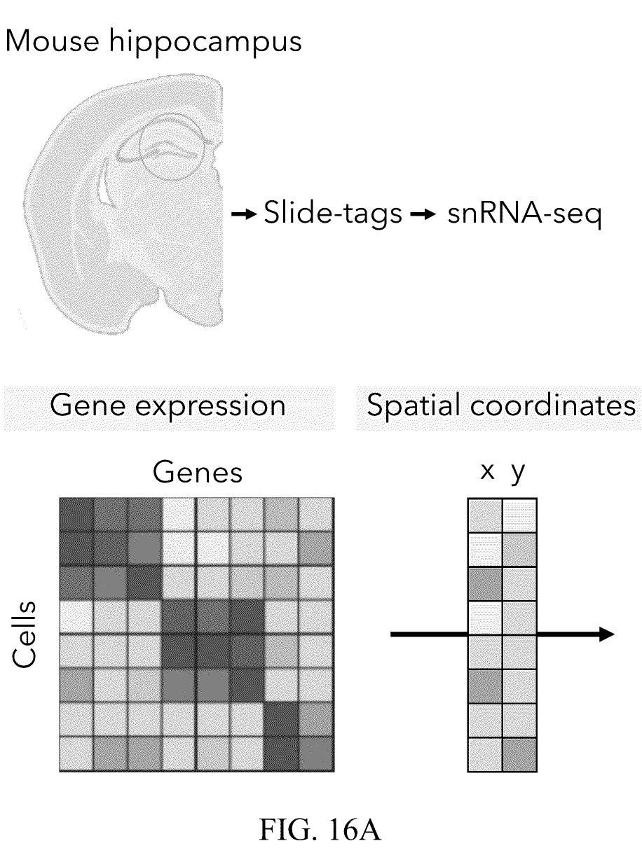

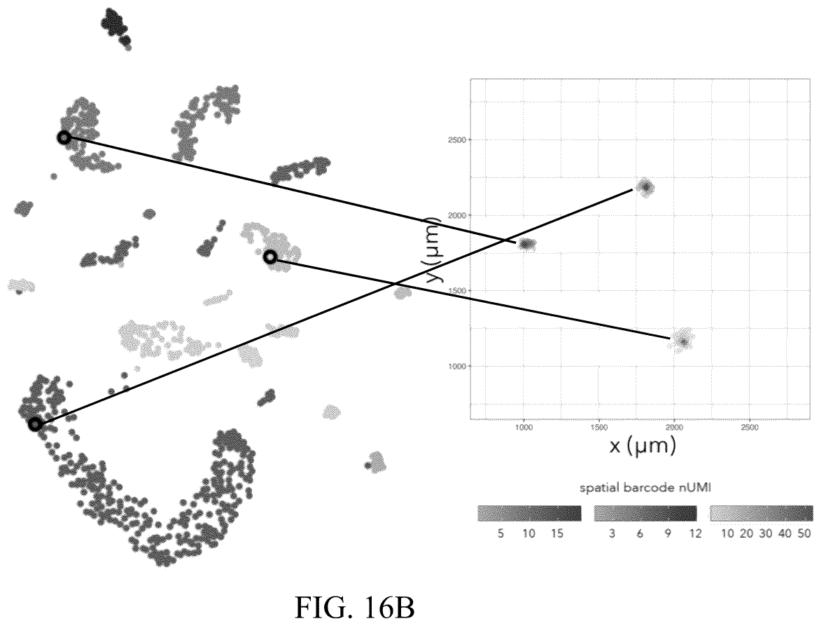

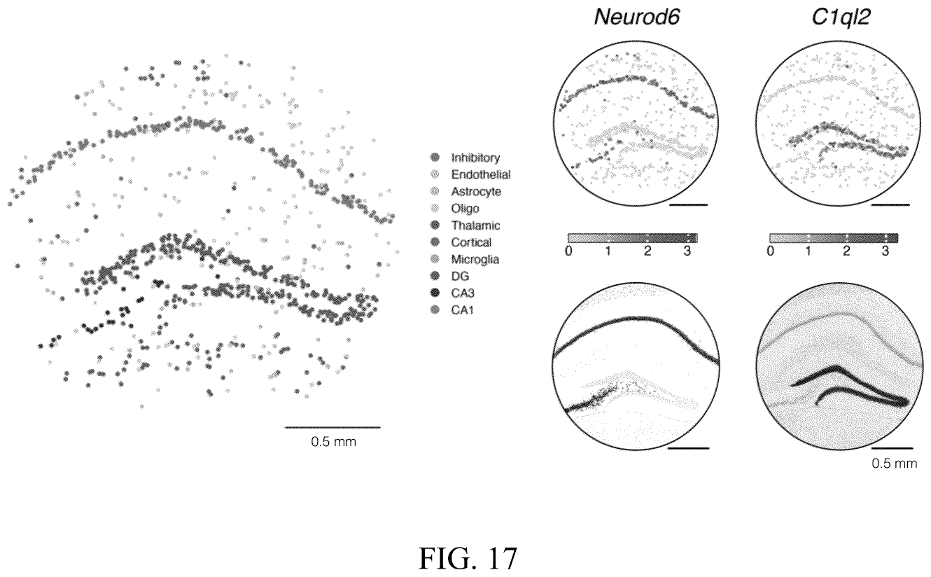

Slide-tags can be used with the 10×single cell sequencing technologies (FIG. 15). In order to benchmark slide-tags, Applicants applied it to profile the mouse hippocampus, which has a highly stereotyped architecture, allowing for clear assessment of the technology's performance (FIG. 16). The standard output from a snRNA-seq experiment is a cells x gene matrix, and from this Applicants constructed dimensionality reduction plots to highlight cell type populations, which are shown here by their shading. Using slide-tags, each of these cells has an associated cluster of spatial barcodes. Applicants then took the weighted center of these points to get the cell's precise location. This allows each slide-tags experiment to have an accompanying spatial coordinate matrix. Slide-tags enables single-nucleus spatial transcriptomics in the mouse hippocampus (FIG. 17). Slide-tags recapitulates expected tissue architecture of the mouse hippocampus after spatially mapping all profiled cells.

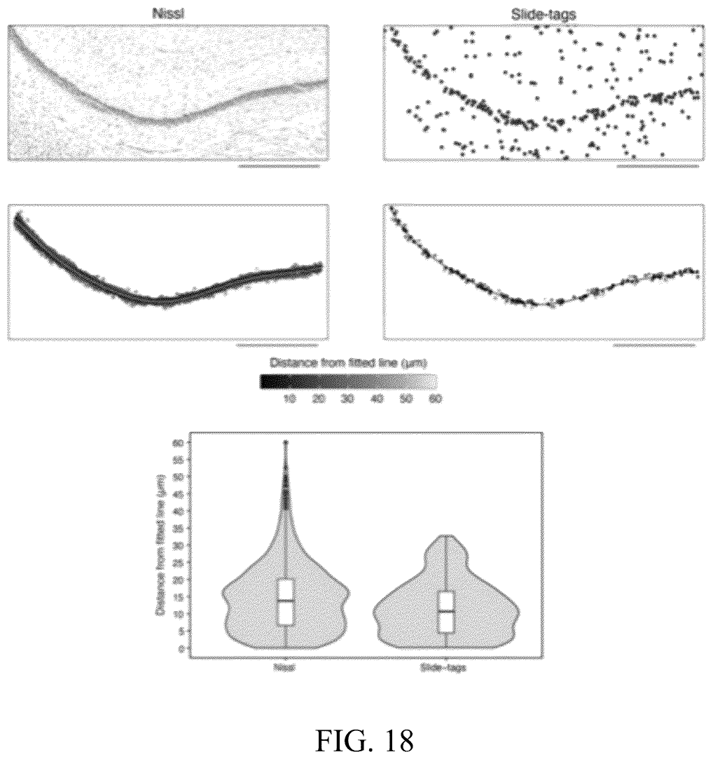

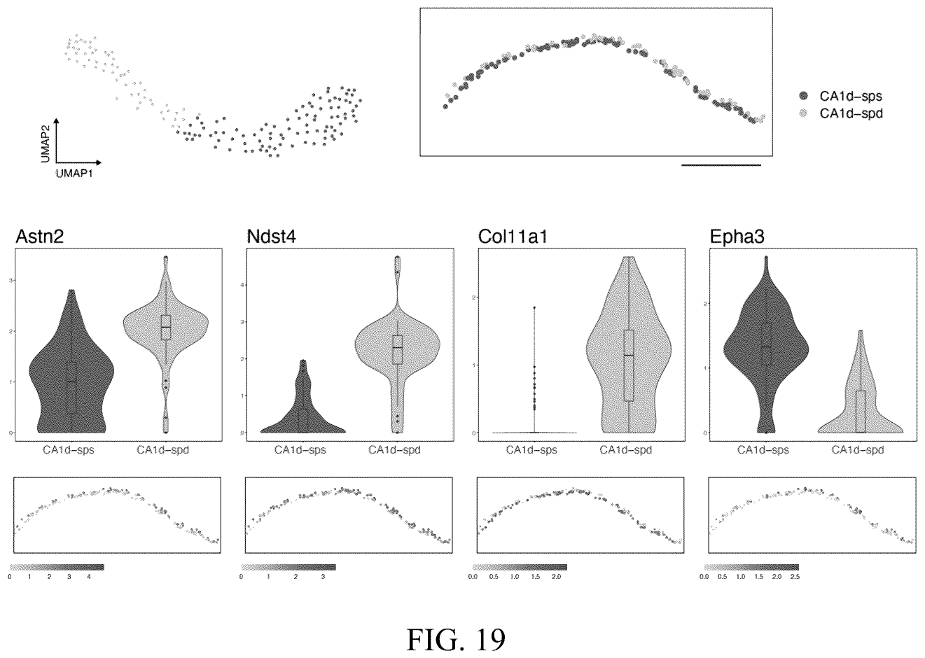

To quantify spatial positioning accuracy, Applicants first compared the width of the hippocampal subfield cornu ammonis area 1 (CA1) in Slide-tags with a Nissl-stained serial section and found that the width of the Slide-tags feature was congruent with the Nissl image (FIG. 18). CA1 is divided into deep and superficial sublayers. Deep and superficial sublayers have distinct gene expression patterns (Dong H W, Swanson L W, Chen L, Fanselow M S, Toga A W. Genomic-anatomic evidence for distinct functional domains in hippocampal field CA1. Proc Natl Acad Sci USA. 2009; 106(28):11794-11799; Cid E, Marquez-Galera A, Valero M, et al. Sublayer- and cell-type-specific neurodegenerative transcriptional trajectories in hippocampal sclerosis. Cell Rep. 2021; 35(10):109229.) Moreover, Applicants could accurately localize sub-cell types in the deep and superficial layers of the CA1 (FIG. 19).

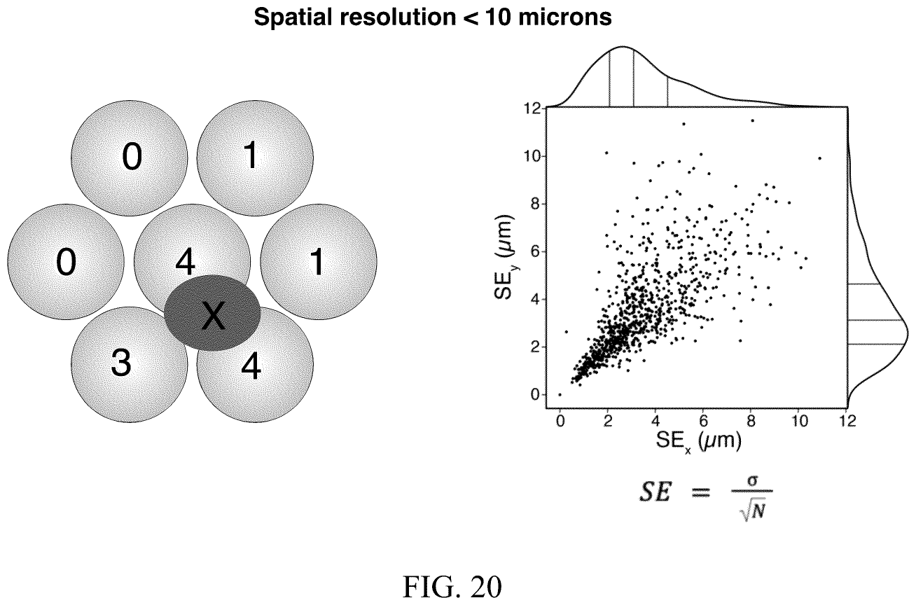

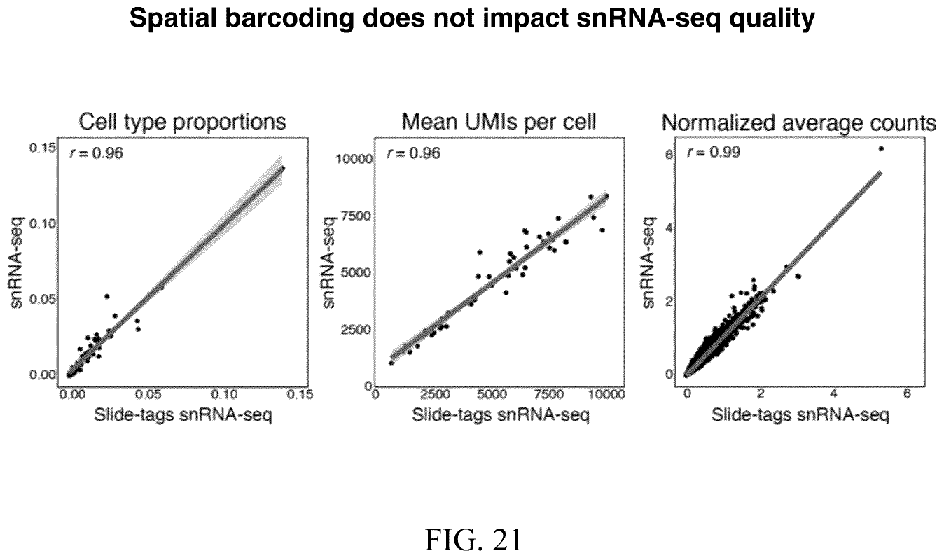

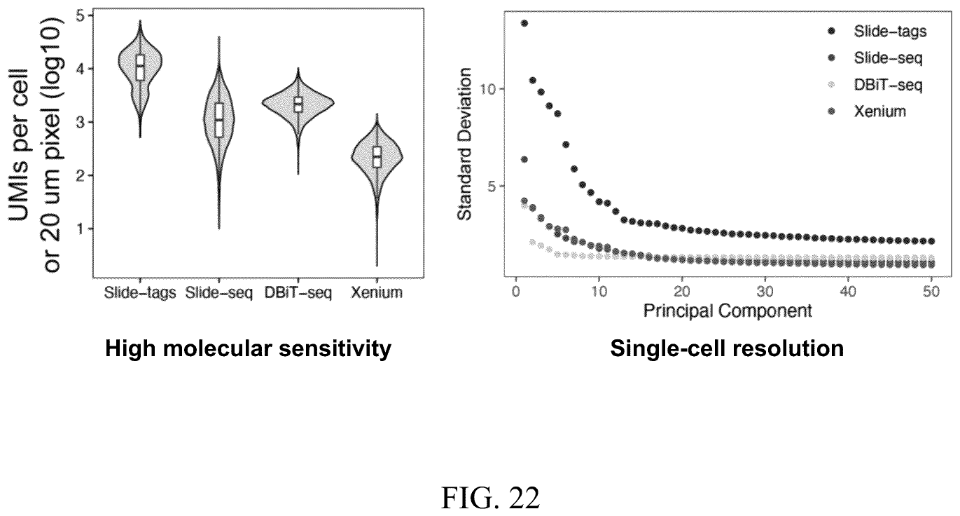

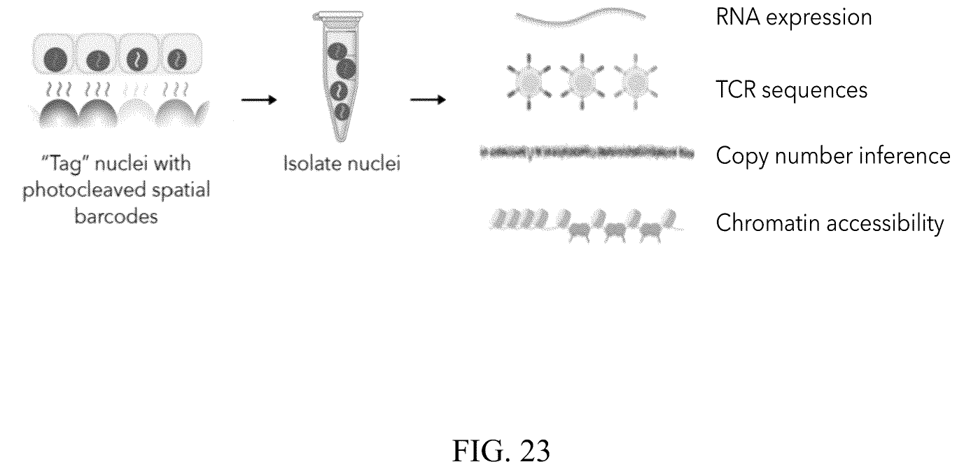

To test if Slide-tags impacts transcriptome data quality, Applicants compared a standard snRNA-seq experiment with Slide-tags followed by snRNA-seq. Applicants found data quality to be nearly indistinguishable in cell type proportions, means UMIs per cell, and average expression across genes (FIG. 21). Also, since each nucleus receives many spatial barcode oligos from many different beads, the spatial resolution is not limited to the 10-micron size of the beads (FIG. 20). For example, cell X received 4 spatial barcodes from two beads, 3 spatial barcodes from another bead, and 1 barcode each from two other beads. Using this information, the location of cell X can be determined within less than 10 microns. With Slide-tags, Applicants estimate the spatial resolution to be less than 10 microns, somewhere between 3 and 4 microns (FIG. 20). Slide-tags generates high quality spatial single cell data that outperforms prior spatial methods (FIG. 22). Slide-tags is compatible with existing single nuclei sequencing technologies as well as analyses tools (FIG. 23). Applicants have demonstrated this with the applications shown.

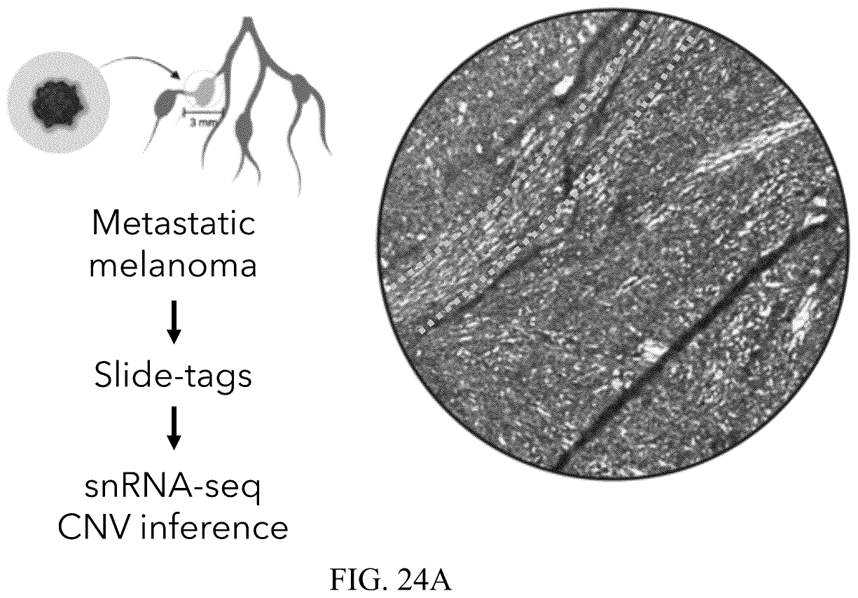

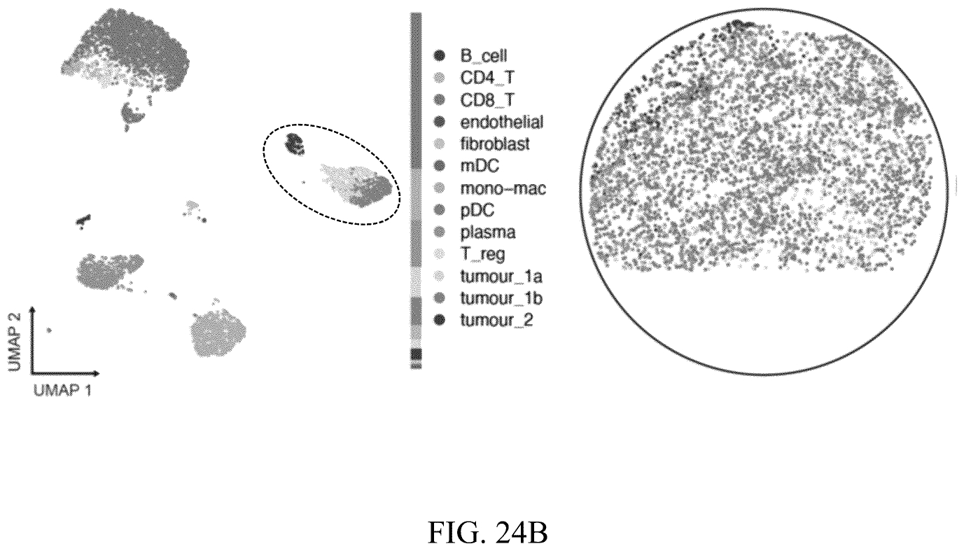

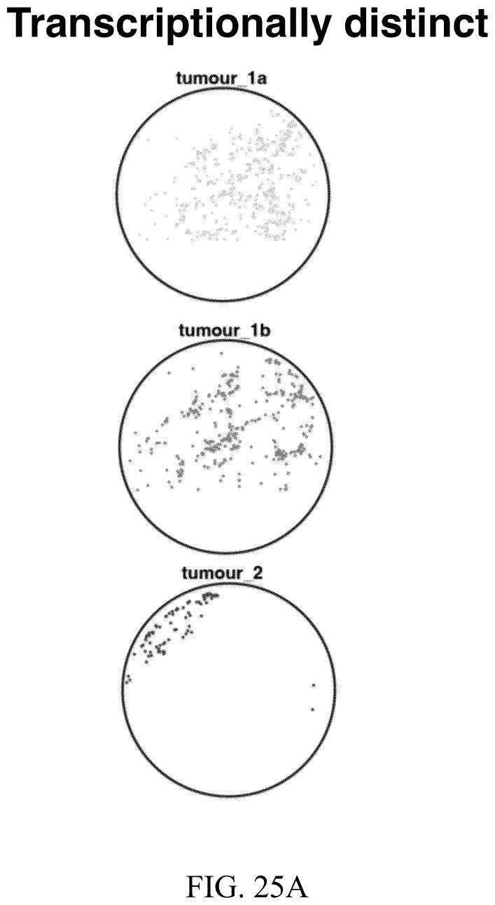

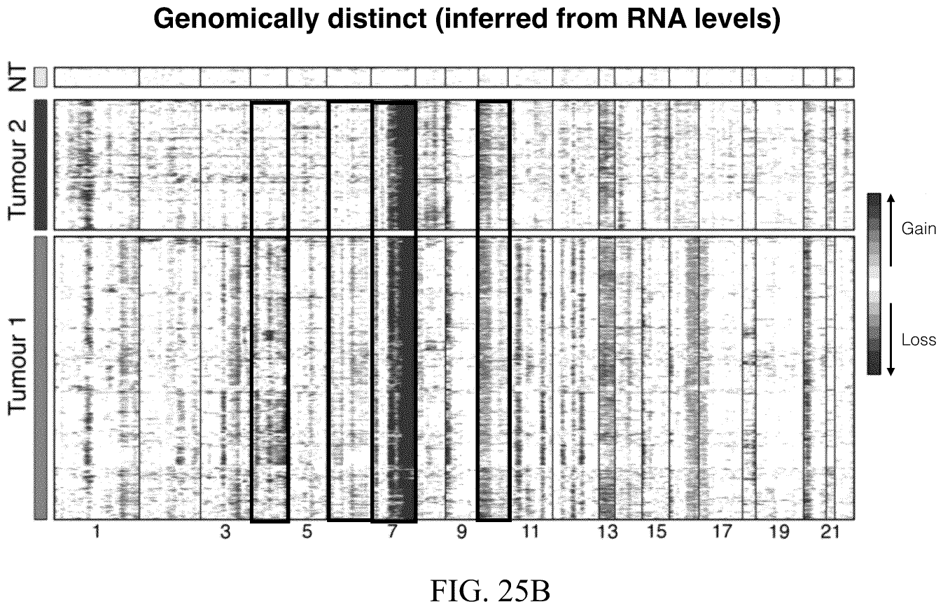

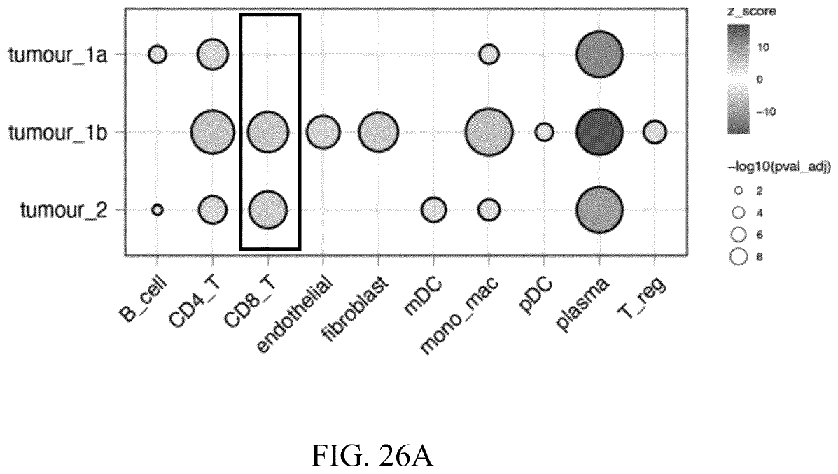

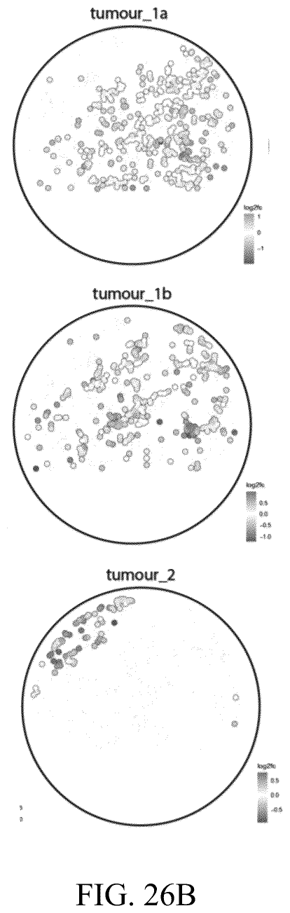

Applicants applied slide tags to a human metastatic melanoma sample (FIG. 24). The H&E image shows two histopathological lobes, divided by a line. As before, Applicants can identify transcriptomic clusters, and here saw 3 clusters of tumor cells, but with slide-tags, these clusters are located. Applicants observed that the profiled cancer cells had two transcriptionally distinct subpopulations. Applicants mapped these subpopulations in space and found them to spatially segregate (FIG. 25A). Applicants asked if there were cytogenetic differences between the subpopulations and used inferCNV, a method to infer copy number variation from transcriptome data. Applicants found many shared copy number variations between the subpopulations, but also found some inferred alterations unique to each subpopulation (FIG. 25B). Using the single-cell data, Applicants investigated how this heterogeneity may relate to cell-cell interactions that these tumor clusters form. Applicants calculated changes in the proportion of neighbors that these tumor cells have and identified CD8 T cells as enriched in the neighborhood of tumor 2, depleted in tumor 1b and neither enriched nor depleted in tumor 1a (FIG. 26A). Applicants show this spatially on a single cell basis when the enrichment in the fraction of CD8_T cells is plotted within each tumors neighborhood (FIG. 26B).

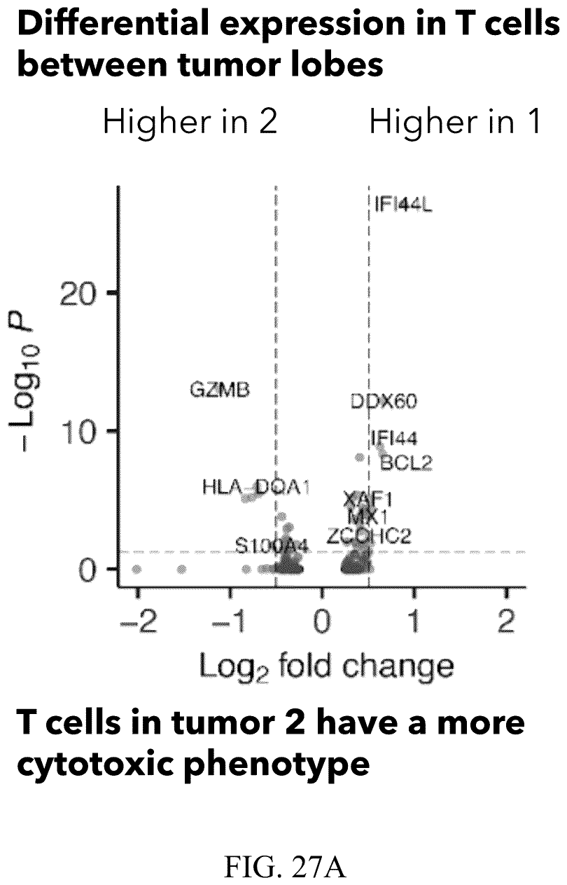

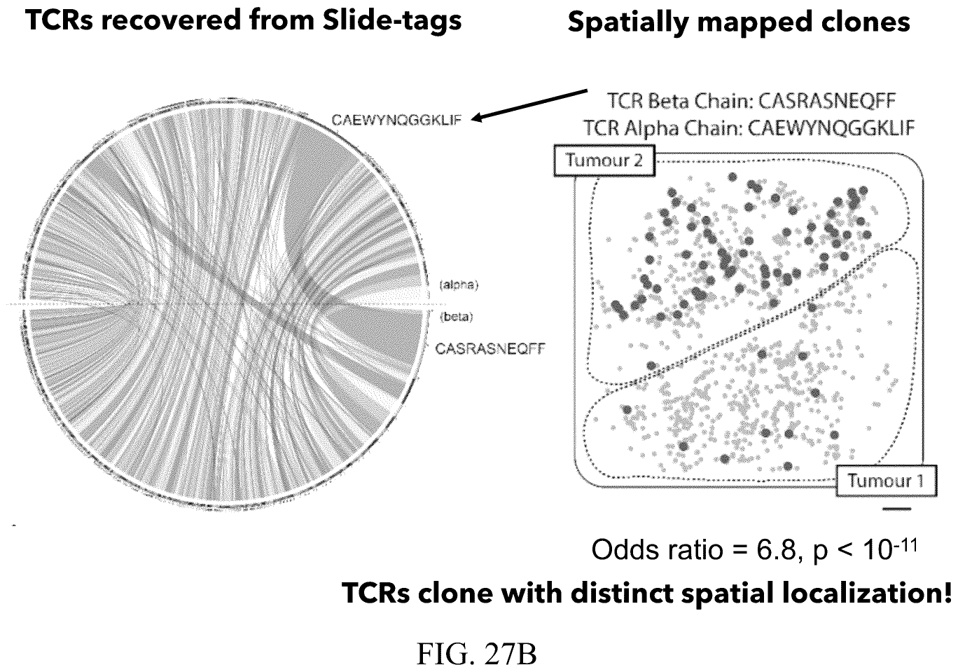

Applicants asked what were the T cell states and receptors in the different neighborhoods. Applicants observed that T cells in tumor 2 have a more cytotoxic phenotype (i.e., cell state) (FIG. 27A). Applicants sequenced TCR receptors from slide-tags and observed that the TCR clones had distinct spatial localization (FIG. 27B). Slide-tags is compatible with sequencing TCR receptors from 3′ single-cell libraries (Liu S, Iorgulescu J B, Li S, et al. Spatial maps of T cell receptors and transcriptomes reveal distinct immune niches and interactions in the adaptive immune response. Immunity. 2022; 55(10):1940-1952.e5).

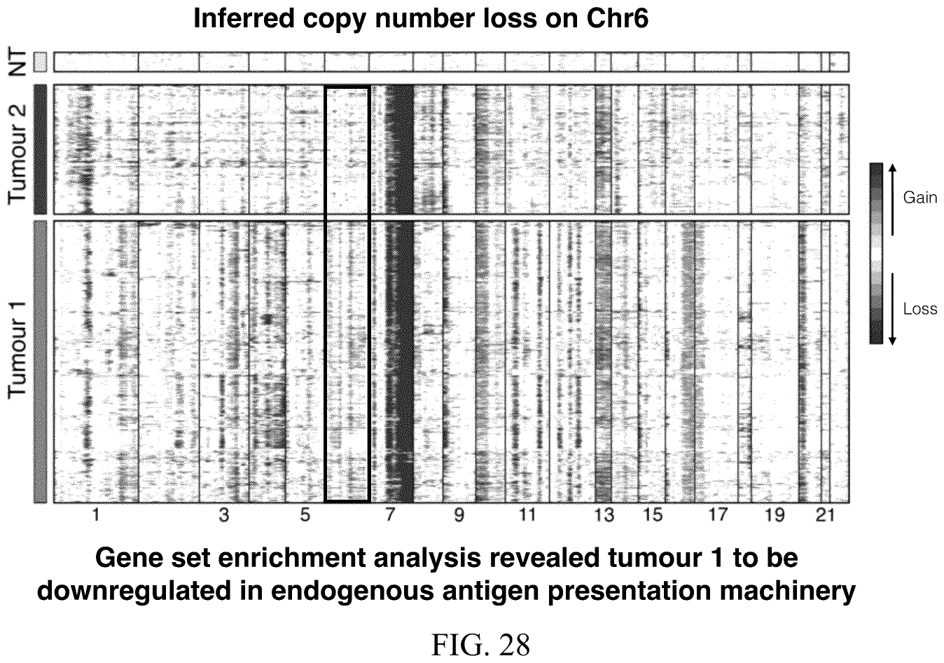

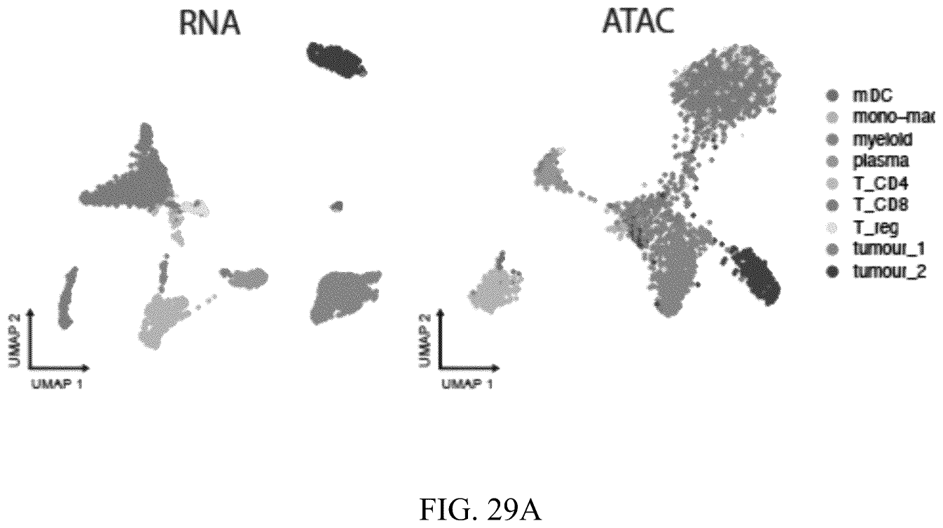

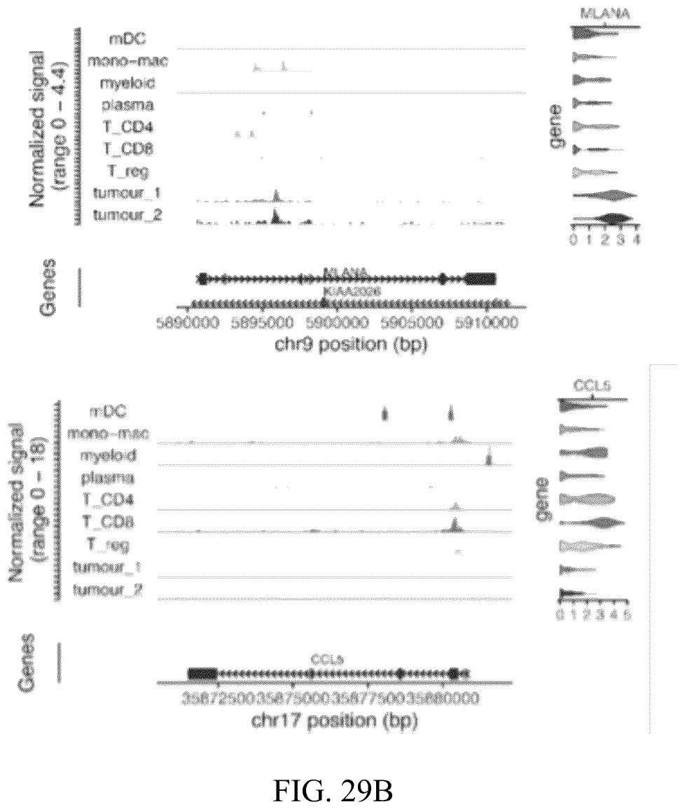

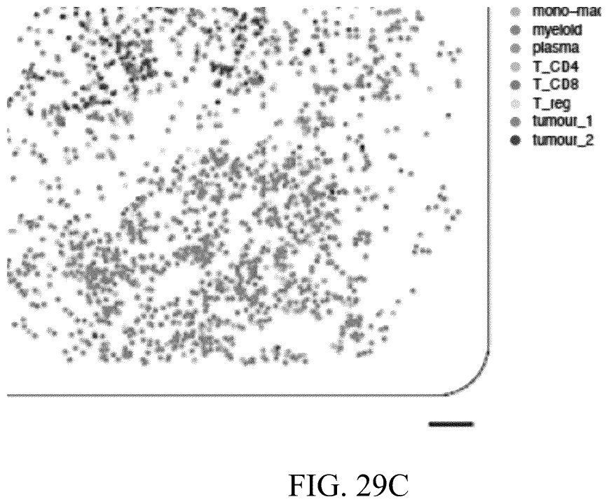

Applicants observed that there is a difference in T cell infiltration and states between tumour 1 and tumour 2. Applicants hypothesized that the HLA locus is downregulated with a copy number loss on chr6 of tumour 1, but not tumour 2. Applicants observed inferred copy number loss on Chr6 (FIG. 28). Further, gene set enrichment analysis on differentially expressed genes between the tumour populations revealed tumour 1 to be downregulated in endogenous antigen presentation machinery. Slide-tags multiome recovers expression and epigenomic profiles (FIG. 29A-C).

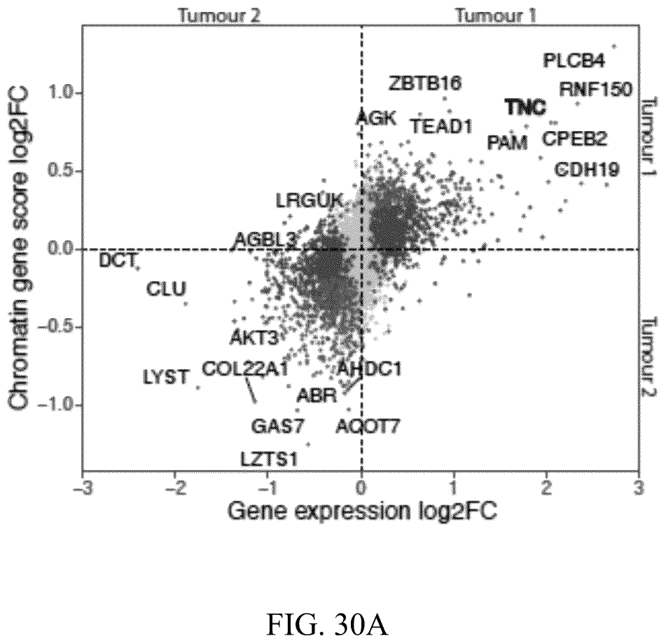

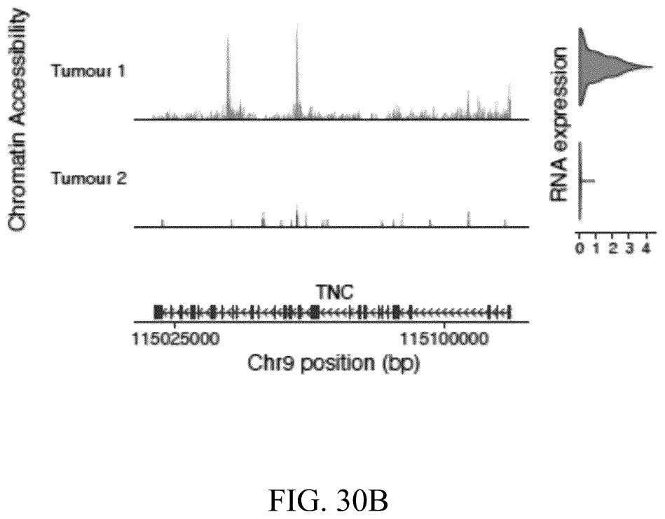

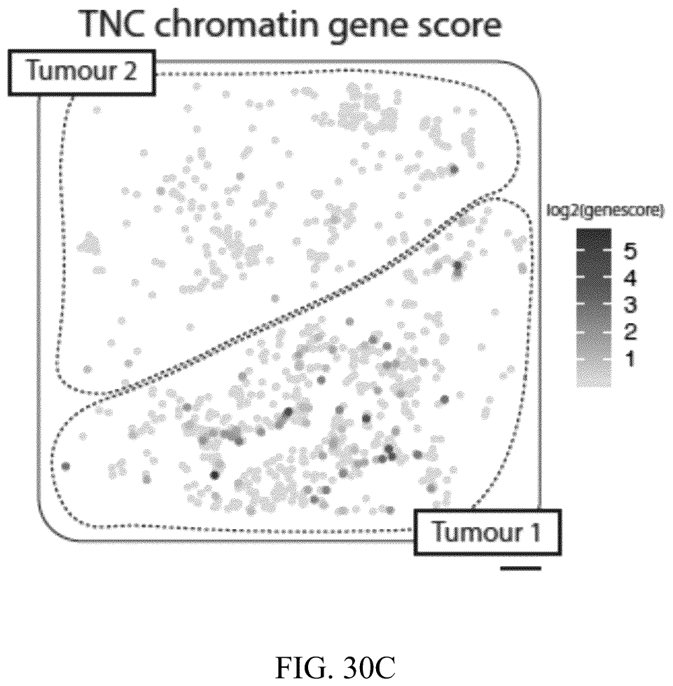

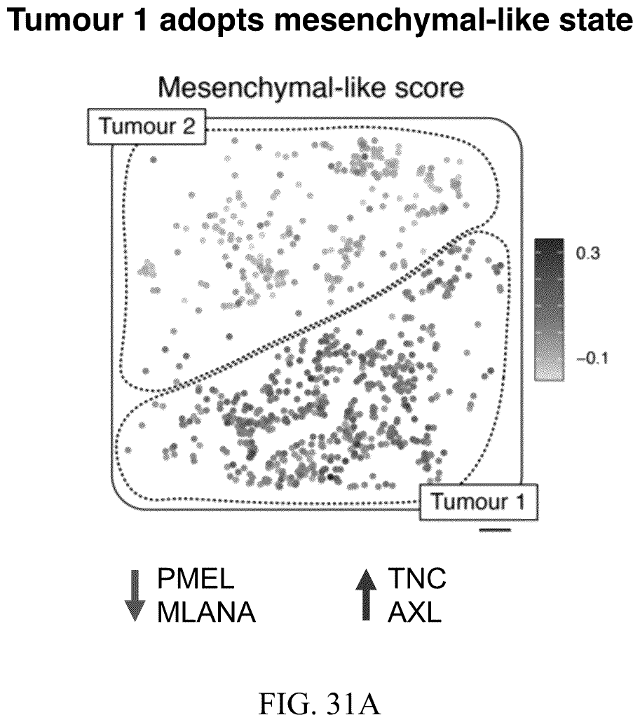

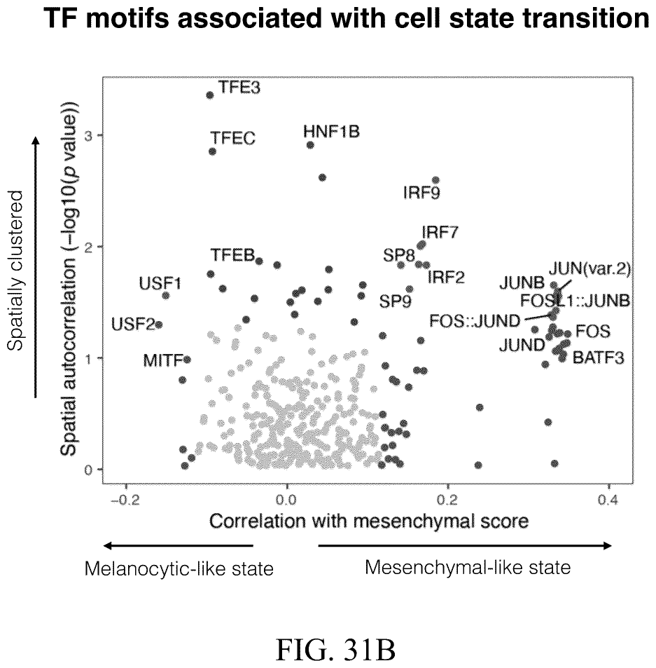

An alternative hypothesis is that melanoma cells in tumour 1 are undergoing a cell state transition, or dedifferentiation accompanied by a loss of endogenous antigen expression. Applicants observed in the RNA and ATAC data together genes both differentially expressed and differentially accessible between the two tumour populations and found TNC, a gene associated with cell state transitions towards more invasive phenotypes in melanoma (FIG. 30A-C). Applicants hypothesized that tumour 1 is losing its melanocytic identity and associated endogenous antigens, resulting in a more suppressive T cell microenvironment. Applicants scored tumour cells on expression of mesenchymal-like cell state genes (FIG. 31A). Applicants found, as postulated, that tumour 1 was enriched in a more mesenchymal-like phenotype compared with tumour 2 and was beginning to lose melanocyte markers like PMEL and MLANA, while gaining TNC and AXL expression. Next, Applicants referred back to the ATAC data in these same nuclei to identify transcription factor motifs associated with this mesenchymal-like state. Applicants found transcription factors like FOS, JUN, and IRF9 were positively correlated with mesenchymal-like scores, while TFs like MITF were negatively correlated (or more correlated with a melanocytic-like score) (FIG. 31B). Finally, mapping these TF motif scores in space, Applicants found that some of the TF motifs correlated mesenchymal-like cell state spatially cluster together. This is suggestive of tumour microenvironmental pressures or epigenetic inheritance through cell division.

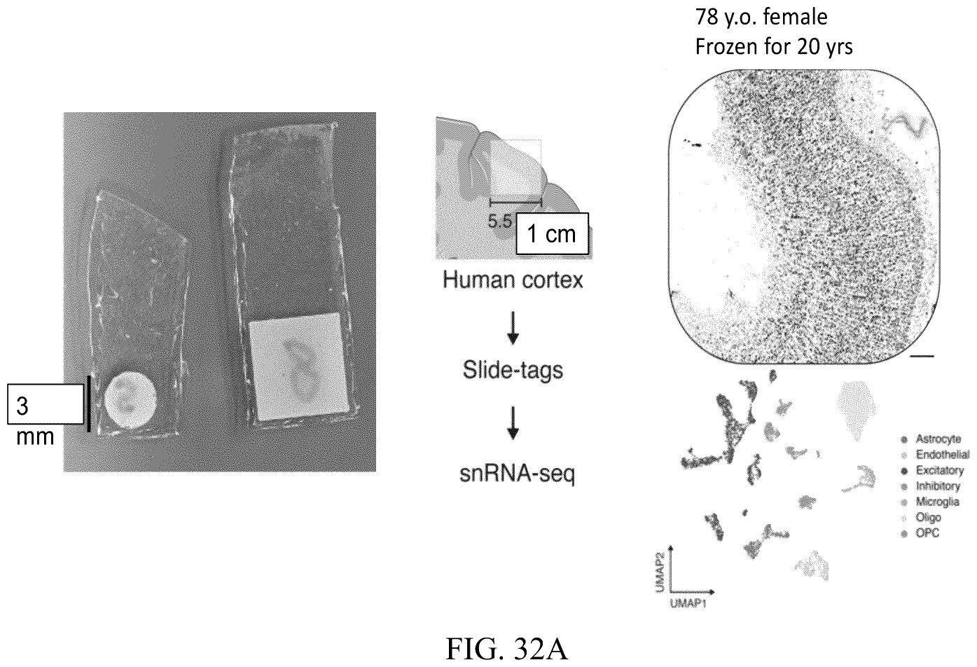

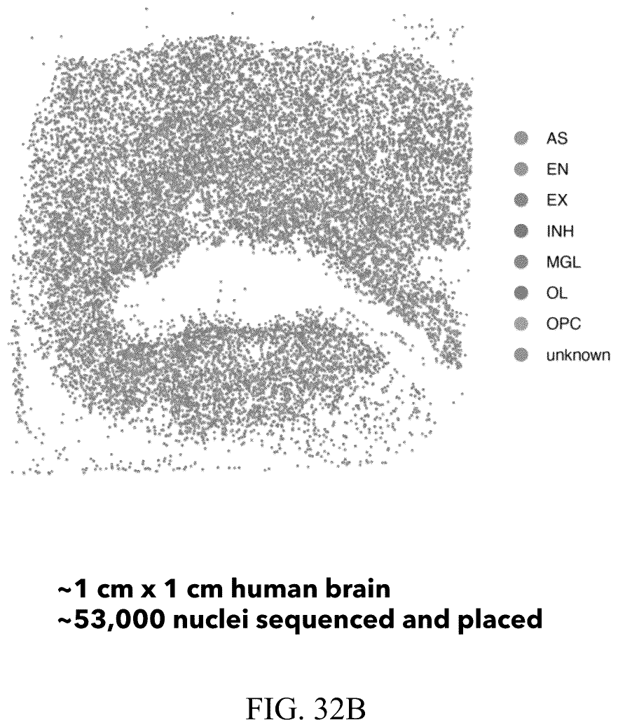

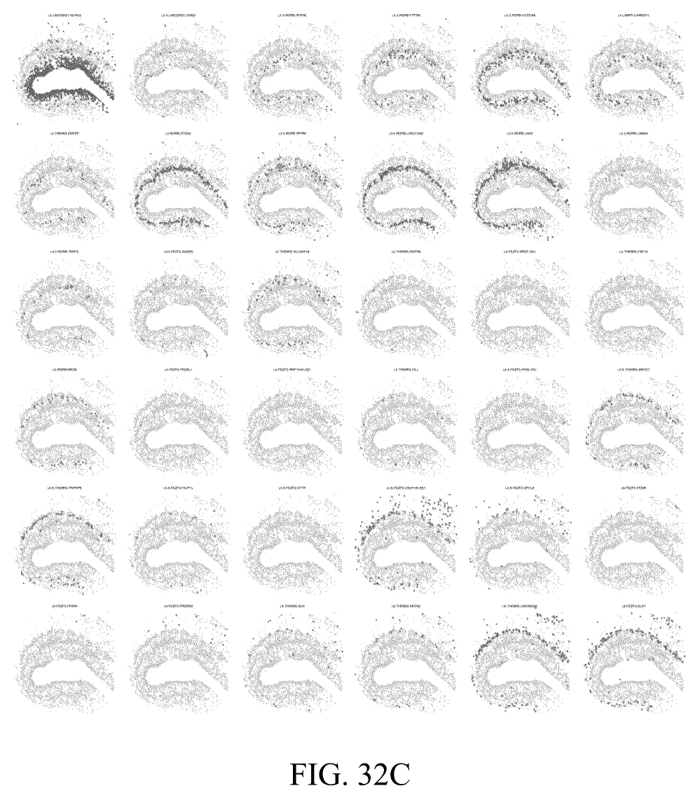

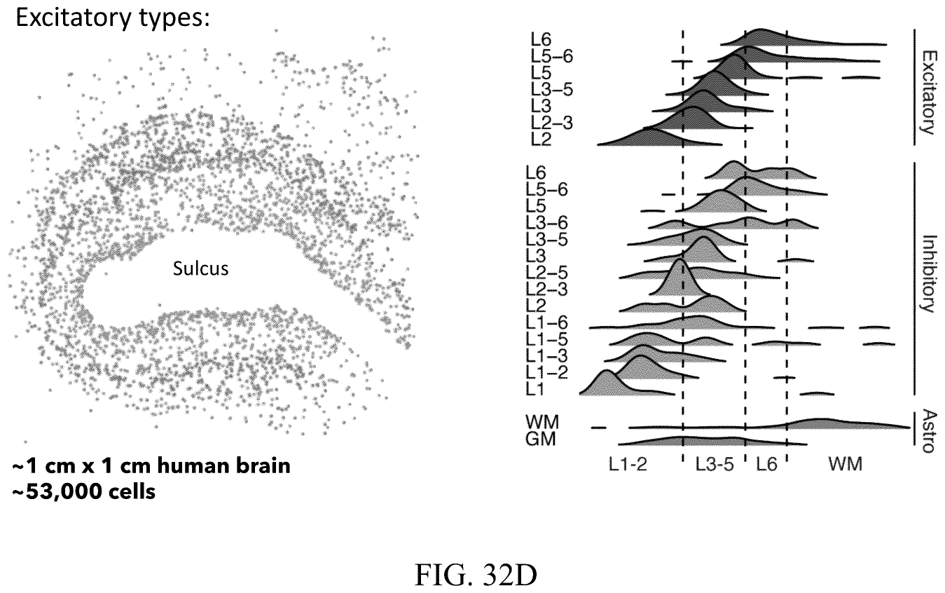

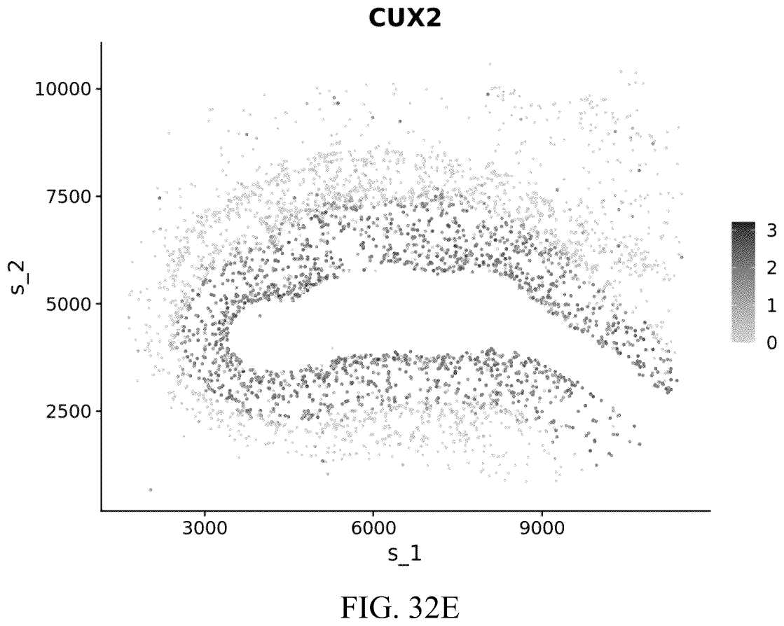

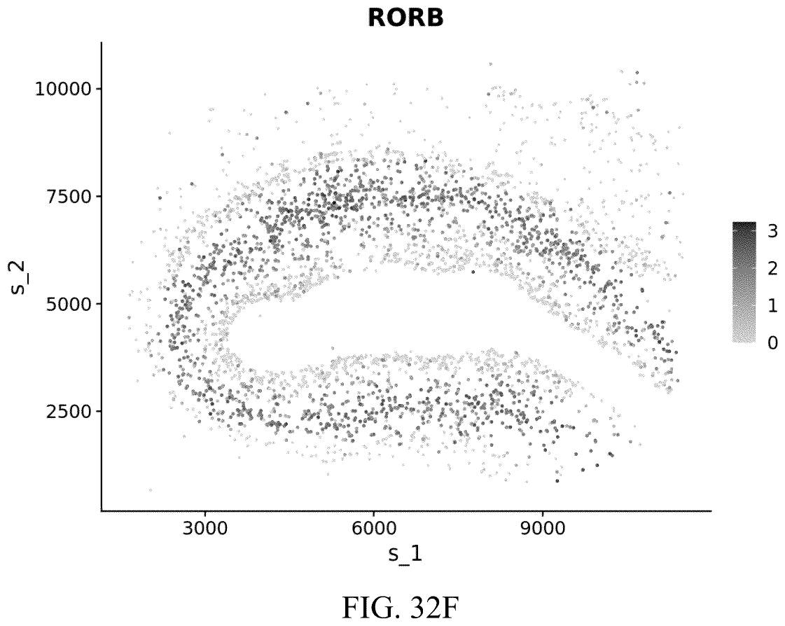

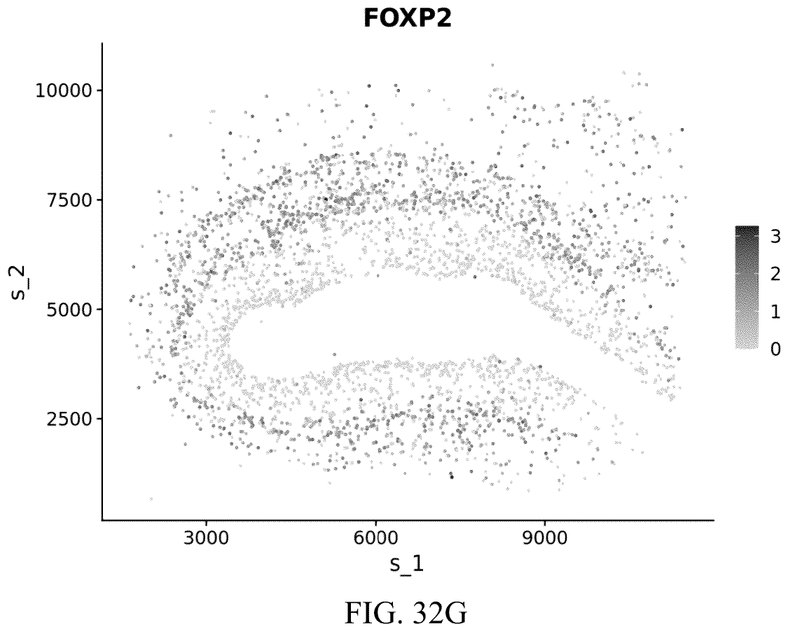

Human tissues often pose unique challenges to technical development. The human brain for example has more sparsely packed nuclei, and so a change of scale in the arrays used for barcoding is required. Applicants performed slide-tags on a 5.5×5.5 area of the human prefrontal cortex where there is a well characterized laminar structure (FIG. 32). Applicants sequenced a total of 14,000 nuclei, of which Applicants were able to place 6500 in space. Applicants recovered all known cortical layered types (see, e.g., Bakken T E, Jorstad N L, Hu Q, et al. Comparative cellular analysis of motor cortex in human, marmoset and mouse [published correction appears in Nature. 2022 April; 604 (7904):E8]. Nature. 2021; 598(7879):111-119).

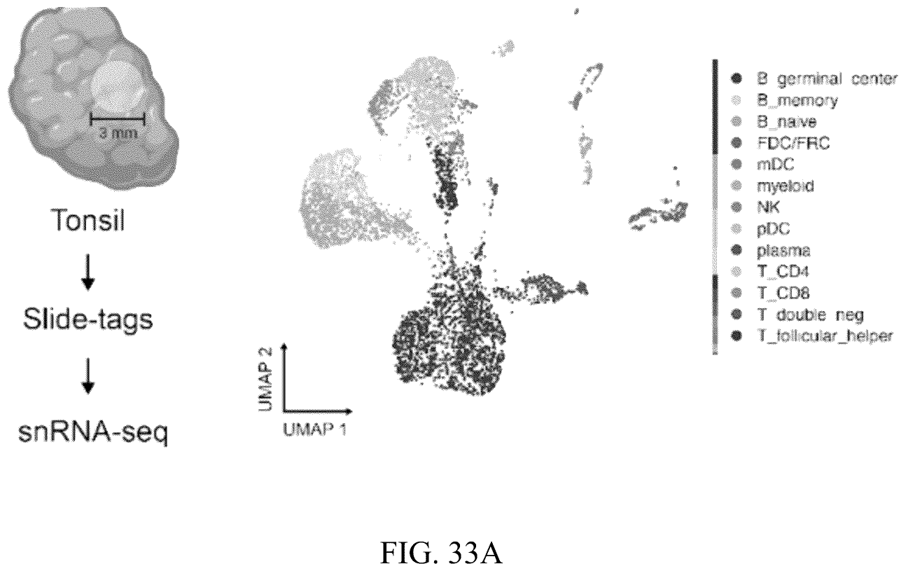

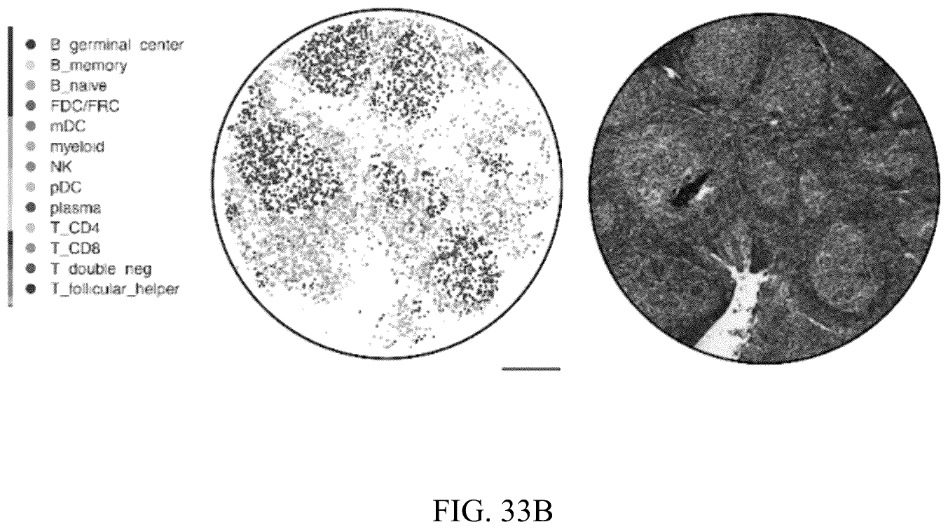

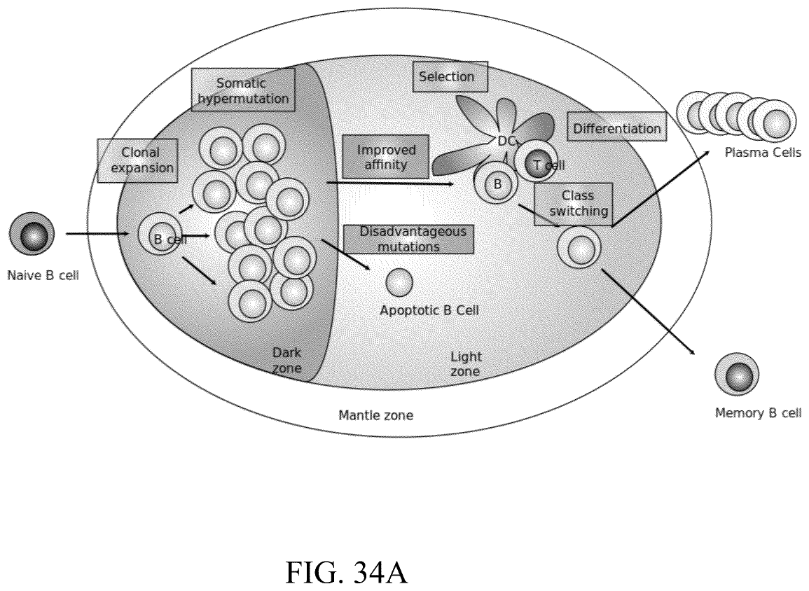

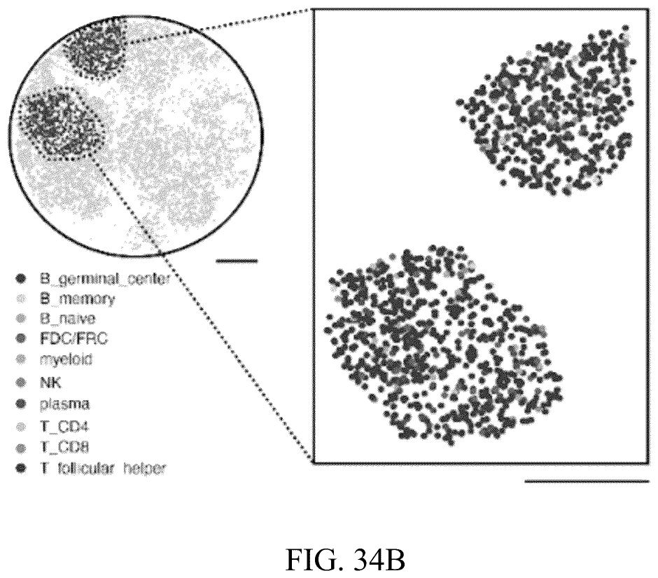

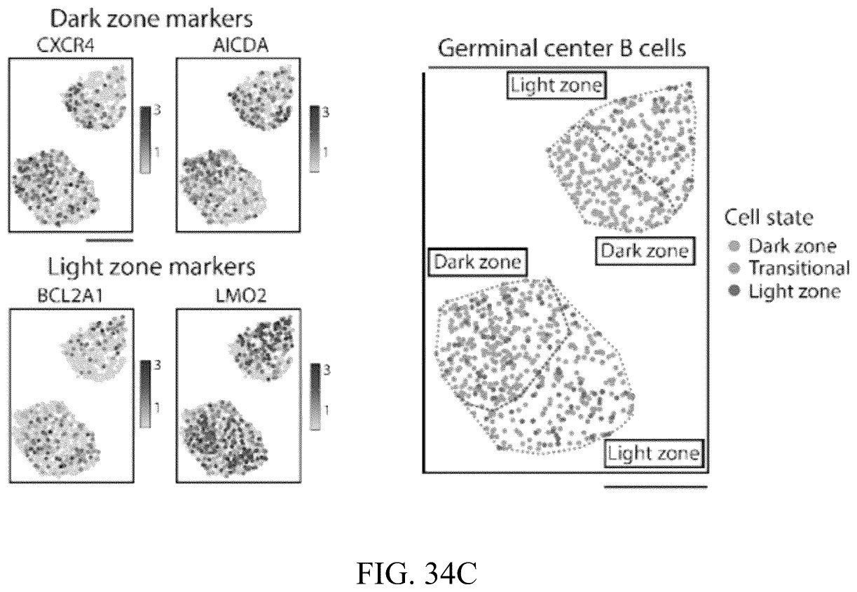

The human tonsil is a highly dynamic dense organ where there are many simultaneous cellular differentiation events happening. The human tonsil includes germinal centers where B cell maturation happens. Memory B cells excite the germinal centre whereas naïve cells are excluded from the germinal centres. Tonsils have rich immune receptor repertoire information. The tonsil is traditionally hard to profile with spatial tools as the cell types are closely juxtaposed and small. The utility of using slide-tags is shown (FIG. 33). Applicants used spatially varying genes to identify dark zone and light zones in germinal centers (FIG. 34).

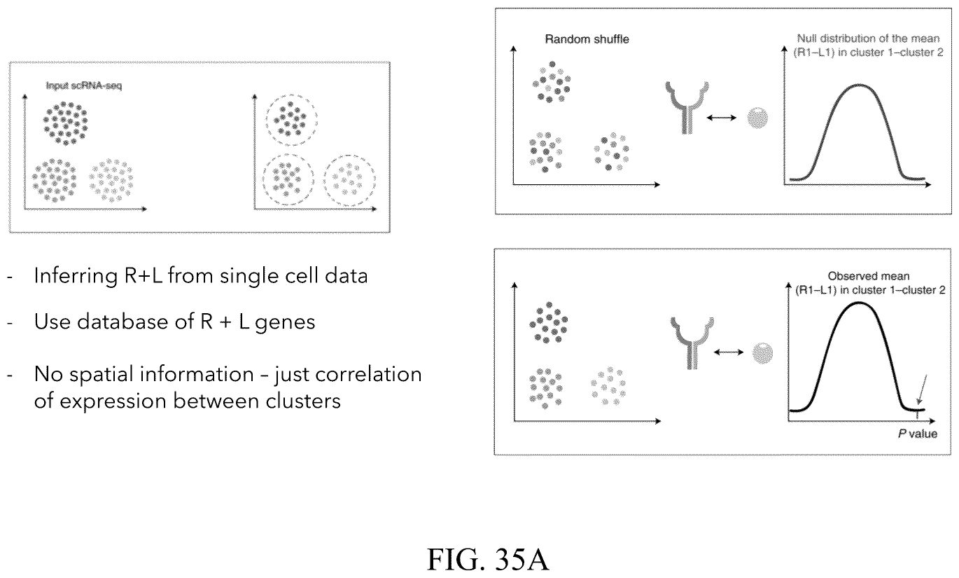

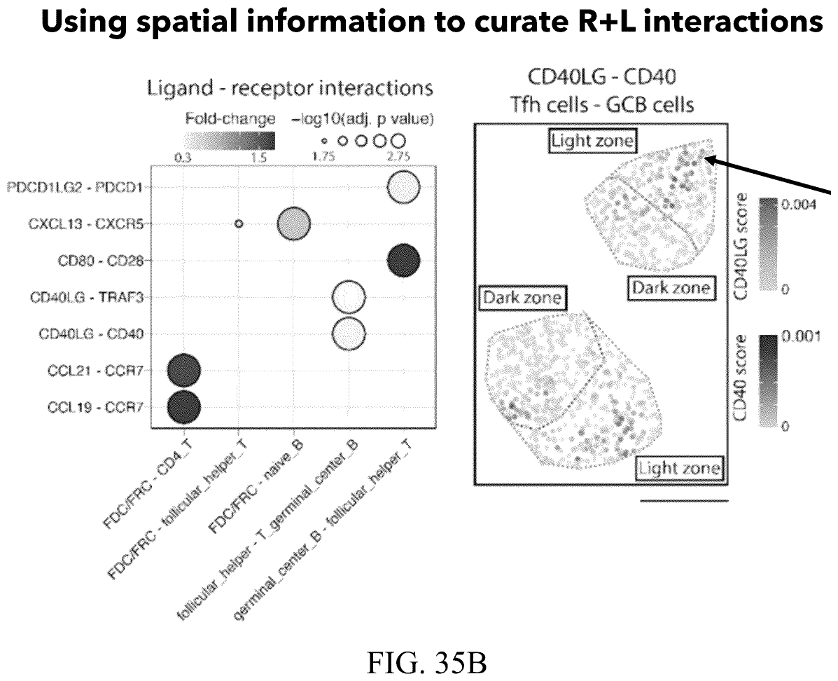

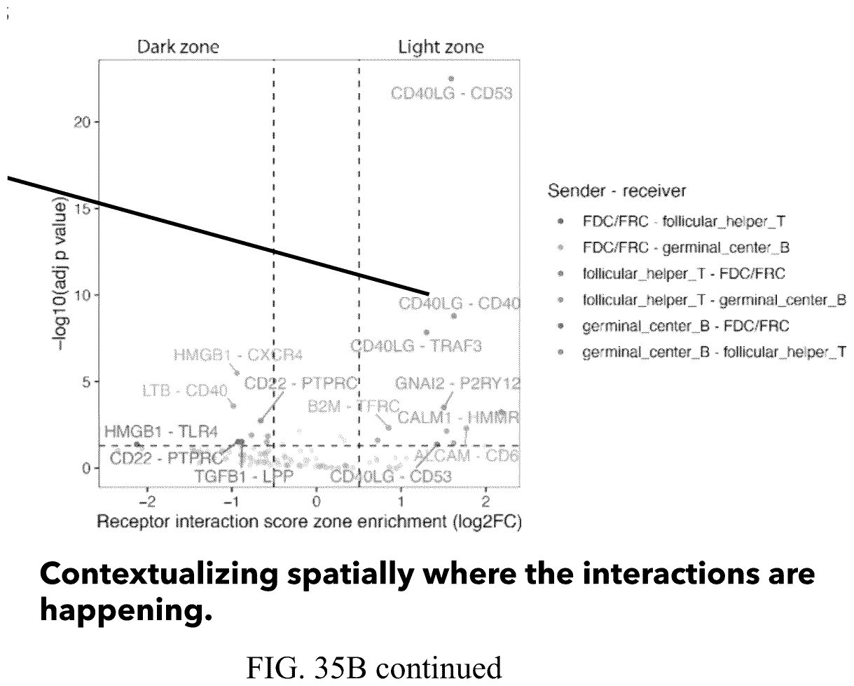

Slide-tags can be used to nominate spatially significant receptor ligand interactions (FIG. 35). Prior methods of inferring receptor ligand interactions using single cell data cannot determine whether the single cells are in spatial proximity (FIG. 35A). Using spatial information to curate receptor ligand interactions in germinal centers allows for single cells in proximity that express ligand receptor pairs to be identified (FIG. 35B). For example, single cells expressing CD40LG and CD40 in the light zone.

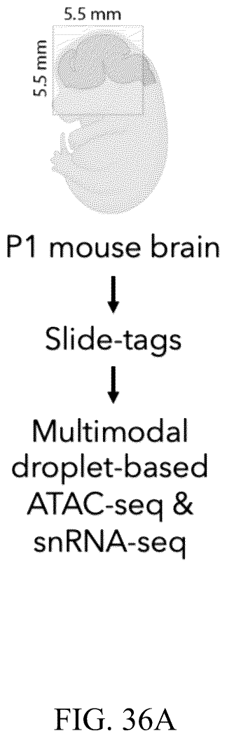

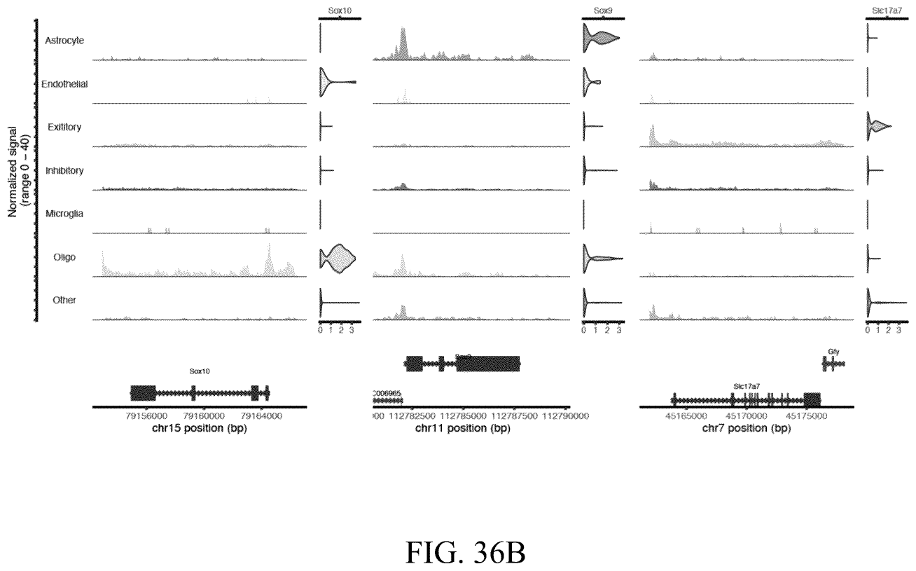

Applicants next explored whether spatial multiomic sequencing can be performed, by testing both ATAC and RNA-seq on the same single-cells, using slide-tags (FIG. 36). Applicants profiled a region of the mouse p1 brain (FIG. 36A). Applicants collected high-quality ATAC data, in: TSS enrichment, fragment lengths corresponding to nucleosome peaks, and high counts of unique fragments per cell (FIG. 36B).

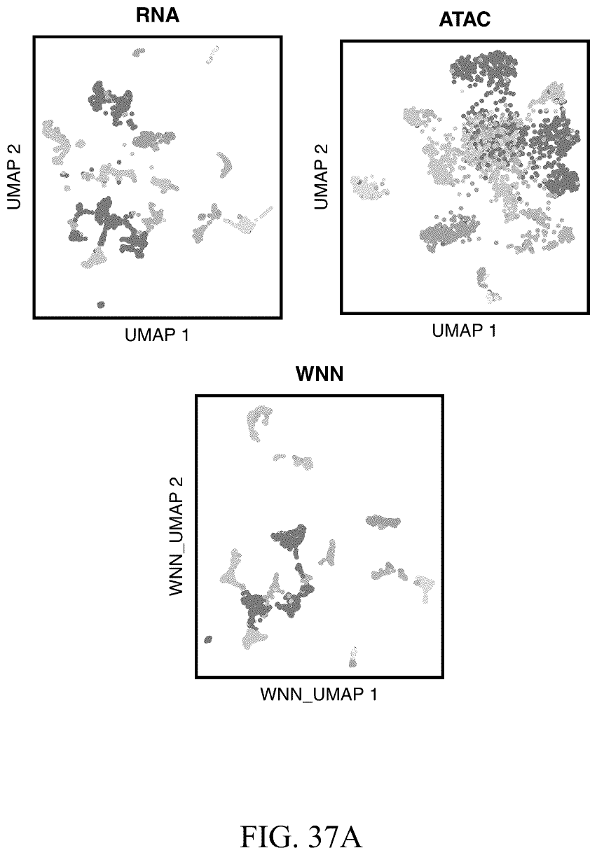

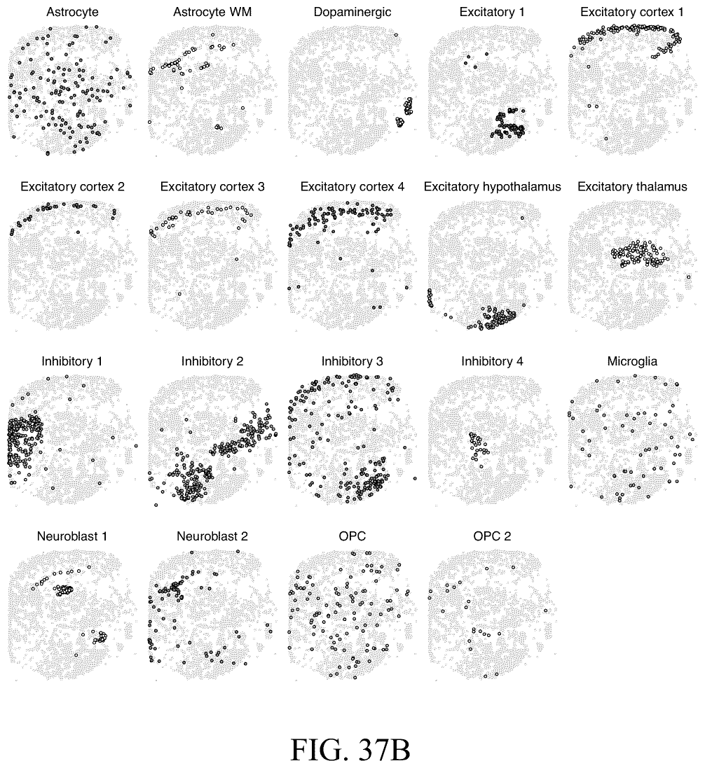

Applicants can both embed cells by their RNA expression and ATAC peak profiles, but now Applicants can also use the combined variation (WNN) to better inform cell clustering (FIG. 37A). Applicants plotted these clusters in space, which reveals many spatially defined clusters (FIG. 37B).

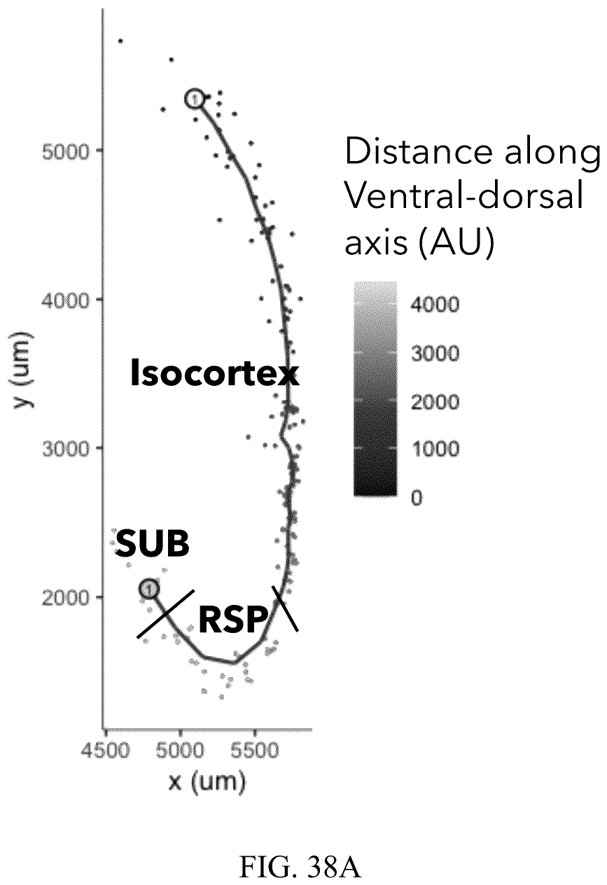

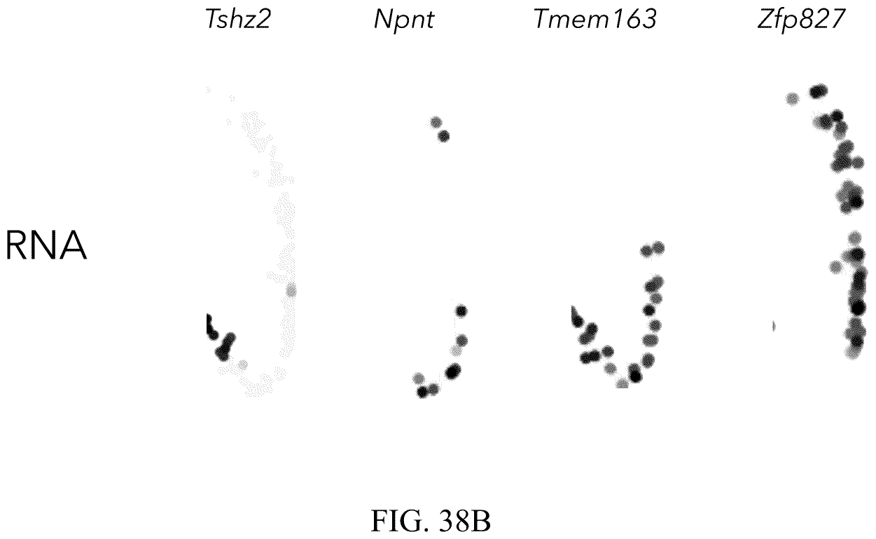

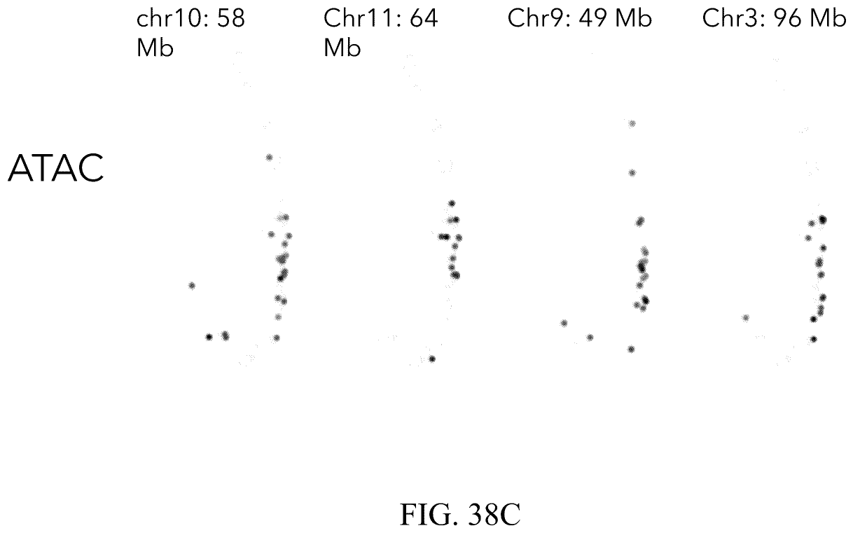

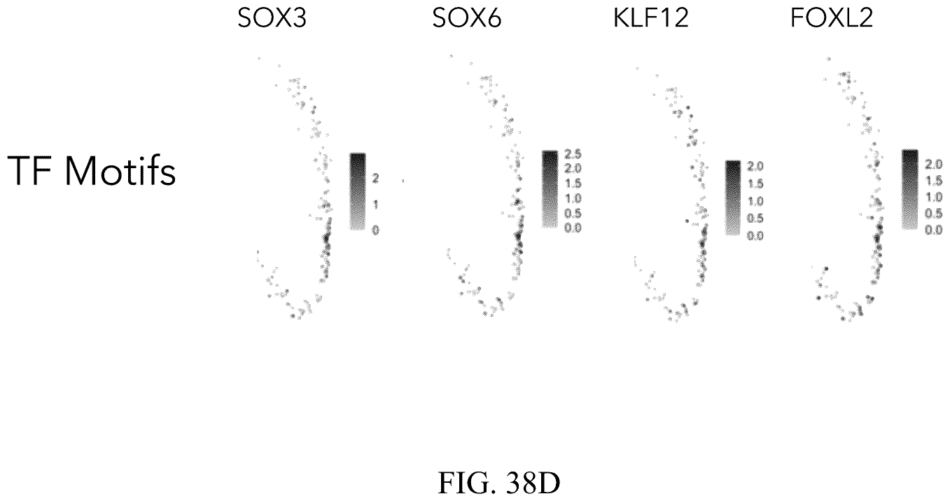

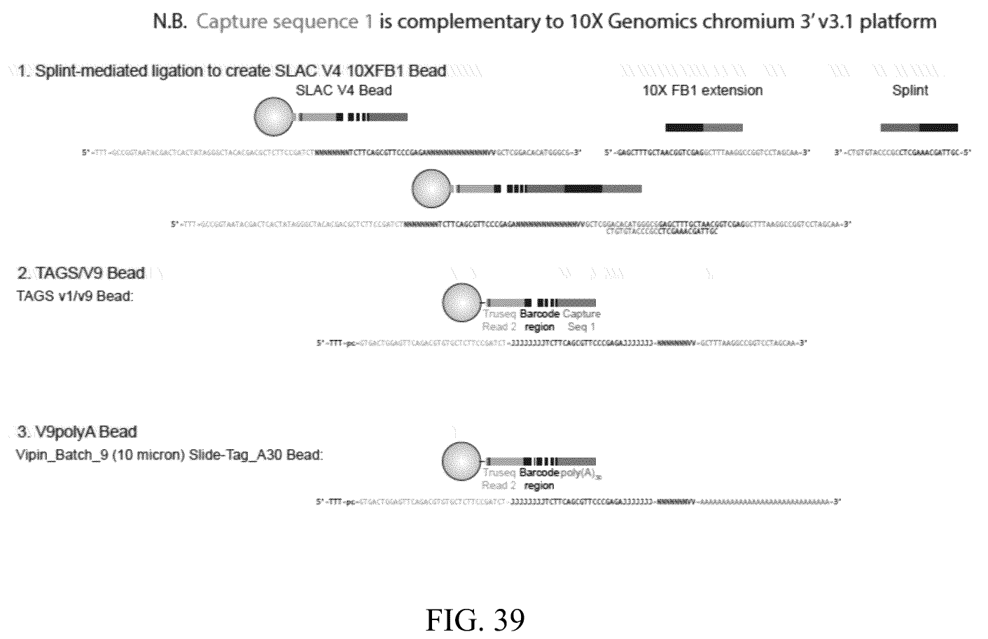

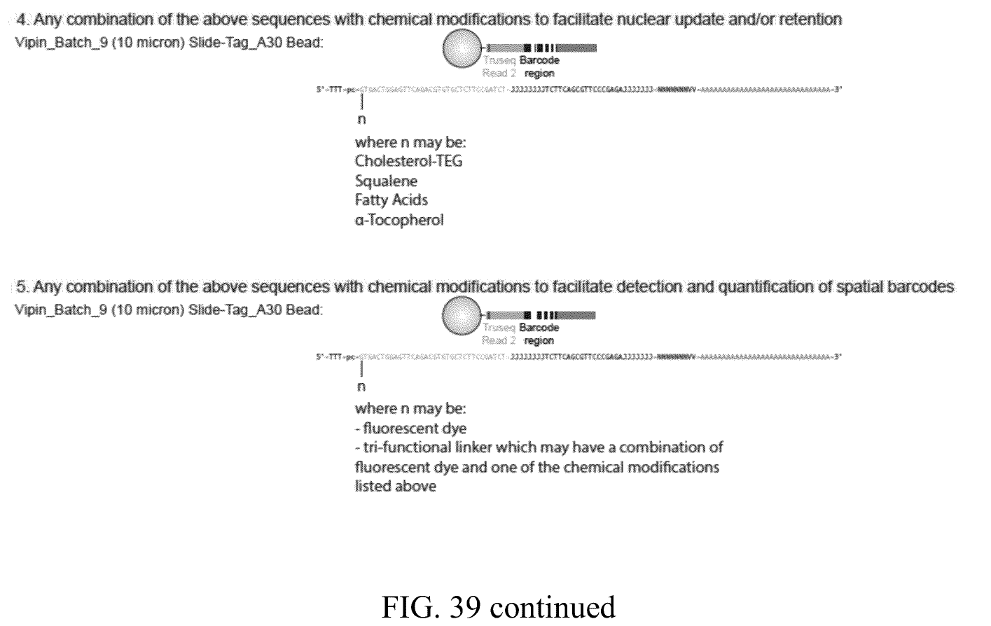

Applicants used slide-tags for the identification of spatially confined transcriptomic and epigenetic features in developing L 2/3 (FIG. 38). Applicants zoomed in on one of these clusters which belonged to the outer layer of the developing cortex, where layers have been defined but the regions along this layer are still being fully defined. Applicants used the RNA data by performing autocorrelation analysis to identify these broad zones: the isocortex, the retrosplenal cortex, and the subiculum shown with some representative top hits (FIGS. 38A and B). Applicants then asked the same question in the ATAC data and discovered that there were peaks identifying a sub-region of the isocortex (FIG. 38C). Applicants sought to understand this further, by identifying transcription factor motifs that vary along this axis and discovered 4 transcription factors with 3 of these having a known role in cortical development (FIG. 38D).

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. A method of generating spatially tagged nuclei for use in single cell genomics comprising:
a) placing a tissue sample having nuclei on a spatial array, wherein the spatial array comprises nucleic acid sequences comprising spatial barcodes, said spatial barcodes coupled to the spatial array via cleavable linkers, wherein the spatial barcodes are the same for an individual location on the spatial array, but are different for any other location on the spatial array;
b) cleaving the linkers and delivering the spatial barcodes to the nuclei in the tissue sample to create tagged nuclei; and
c) isolating the tagged nuclei from the tissue sample to created isolated tagged nuclei.

2. The method of claim 1, further comprising preparing a single cell genomics sequencing library using the isolated tagged nuclei, wherein nucleic acid sequences each comprising a cell of origin identifying cell barcode sequence capture the spatial barcode from each spatially tagged nucleus to create a combined nucleic acid sequence comprising the spatial barcode and the cell barcode sequence, such that genomics data for each single nucleus can be identified by the cell barcode sequence and the spatial location of the same single nucleus in the tissue sample can be identified by the spatial barcode.

3. The method of claim 1, wherein the spatial barcodes are delivered to the nuclei by diffusion.

4. The method of claim 1, wherein before step (a) the spatial array is sequence verified by in situ sequencing of the nucleic acid sequences comprising spatial barcodes, whereby an index of the spatial barcodes on the spatial array is generated.

5. The method of claim 4, wherein in situ sequencing is performed by sequencing by ligation or sequencing by synthesis.

6. The method of claim 1, wherein the spatial array comprises solid supports fixed at each location on the spatial array.

7. (canceled)

8. The method of claim 6, wherein:
the solid supports are fixed to the spatial array with a vinyl polymer; and
wherein the solid supports are beads.

9-10. (canceled)

11. The method of claim 8, wherein the beads are 50 μm or less, 20 μm or less, 15 μm or less, 10 μm or less, 3 μm or less, or 1 μm or less in diameter.

12. (canceled)

13. The method of claim 1, wherein the linkers are photocleavable, chemically cleavable, or enzymatically cleavable linkers.

14. The method of claim 2, wherein the spatial barcodes comprise poly-A sequences for capture by a cell barcode nucleic acid comprising a poly-T sequence.

15. The method of claim 1, wherein the tissue sample is treated to permeabilize the nuclei.

16-17. (canceled)

18. The method of claim 1, wherein the spatial barcodes further comprise one or more modifications that enhance diffusion to the nucleus.

19. The method of claim 18, wherein the spatial barcodes are modified by addition of one or more lipid or cholesterol groups.

20. (canceled)

21. The method of claim 1, wherein:
the tissue sample is a fresh frozen tissue section,
the tissue sample is a fresh unfixed tissue section, and/or
the tissue sample is a fixed tissue section.

22-23. (canceled)

24. The method of claim 2, wherein the location of each cell in the tissue sample is computationally determined based on sequencing of the single cell genomics sequencing library.

25. The method of claim 24, wherein the cell barcode sequences comprise UMI sequences and the location of each cell in the tissue is determined based on the number of UMIs sequenced for each spatial barcode having the same cell barcode sequence.

26. The method of claim 2, wherein:
the single cell genomics sequencing library is a single nucleus RNA-sequencing library (snRNA-seq),
the single cell genomics sequencing library is a single cell DNA accessibility library,
the single cell genomics sequencing library is a single cell ATAC-sequencing library (ATAC-seq),
the single cell genomics sequencing library is a single cell chromatin immunoprecipitation (ChIP) sequencing library,
the single cell genomics sequencing library is a single cell genome sequencing library,
the single cell genomics sequencing library is a single cell DNA-methylation sequencing library,
the single cell genomics sequencing library is a single cell Hi-C sequencing library,
the single cell genomics sequencing library is a single cell cut-and-tag sequencing library,
the single cell genomics sequencing library is a single cell genome and transcriptome sequencing library (G&T-seq), and/or
the single cell genomics sequencing library is a single cell proteomic library.

27-35. (canceled)

36. A kit comprising:
a spatial array comprising a plurality of solid supports coupled via cleavable linkers to nucleic acid sequences,
said nucleic acid sequences comprising spatial barcodes and a capture sequence,
wherein the spatial barcodes are the same for an individual solid support of the plurality of solid supports, but are different for any other solid support in the spatial array, and
wherein the capture sequence is the same for all of the solid supports.

37-47. (canceled)

48. The method of claim 1, further comprising preparing a single cell genomics sequencing library using the isolated tagged nuclei, further comprising providing nucleic acid sequences each comprising a cell of origin identifying cell barcode sequence and a unique molecular identifier (UMI), wherein the nucleic acid sequences capture the spatial barcode from each tagged nucleus to create a combined nucleic acid sequence comprising the spatial barcode, the UMI, and the cell barcode sequence, such that genomics data for each single nucleus can be identified by the cell barcode and the spatial location of the same single nucleus in the tissue sample can be identified by the spatial barcode.

49. A method of generating spatially tagged nuclei, the method comprising:
a) placing a tissue sample having nuclei on a spatial array, wherein the spatial array comprises individual beads fixed at each location on the spatial array, wherein nucleic acid sequences comprising spatial barcodes and unique molecular identifiers (UMIs) are coupled to the beads via cleavable linkers, wherein the spatial barcodes are the same for an individual bead on the spatial array, but are different for any other bead on the spatial array;
b) cleaving the linkers and delivering the spatial barcode to nuclei in the tissue sample to create tagged nuclei; and
c) isolating the tagged nuclei from the tissue sample.


SEQUENCE LISTING (28 sequences)

SEQ ID NO: 1 - DNA, 109 bp, unidentified
  source 1..109: mol_type = other DNA
tttgccggtaatacgactcactatagggctacacgacgctcttccgatctnnnnnnnntcttcagcgttcccgagannnnnnnnnnnnnnvvgctcggacacatgggcg

SEQ ID NO: 2 - DNA, 42 bp, unidentified
  source 1..42: mol_type = other DNA
gagctttgctaacggtcgaggctttaaggccggtcctagcaa

SEQ ID NO: 3 - DNA, 26 bp, unidentified
  source 1..26: mol_type = other DNA
cgttagcaaagctccgcccatgtgtc

SEQ ID NO: 4 - DNA, 151 bp, unidentified
  source 1..151: mol_type = other DNA
tttgccggtaatacgactcactatagggctacacgacgctcttccgatctnnnnnnnntcttcagcgttcccgagannnnnnnnnnnnnnvvgctcggacacatgggcggagctttgctaacggtcgaggctttaaggccggtcctagcaa

SEQ ID NO: 5 - DNA, 33 bp, unidentified
  source 1..33: mol_type = other DNA
aagcagtggtatcaacgcagagtacatrgrgrg

SEQ ID NO: 6 - DNA, 196 bp, unidentified
  source 1..196: mol_type = other DNA
gtcagatgtgtataagagacagnnnnnnnnnnnnnnnnnnnnnnnnnnnnttgctaggaccggccttaaagcagagcccttgcgacttctcgcccatgtgtccgagcvvnnnnnnnnnnnnnnagagcccttgcgacttctnnnnnnnnagatcggaagagcgtcgtgtagccctatagtgagtcgtattaccggc

SEQ ID NO: 7 - RNA, 112 nt, unidentified
  source 1..112: mol_type = other RNA
ctacacgacgctcttccgatctnnnnnnnnnnnnnnnnnnnnnnnnnnnnttttttttttttttttttttttttttttttvncccatgtactctgcgttgataccactgctt

SEQ ID NO: 8 - DNA, 27 bp, unidentified
  source 1..27: mol_type = other DNA
gcagcgtcagatgtgtataagagacag

SEQ ID NO: 9 - DNA, 22 bp, unidentified
  source 1..22: mol_type = other DNA
ctacacgacgctcttccgatct

SEQ ID NO: 10 - DNA, 22 bp, unidentified
  source 1..22: mol_type = other DNA
aagcagtggtatcaacgcagag

SEQ ID NO: 11 - DNA, 82 bp, unidentified
  source 1..82: mol_type = other DNA
ctacacgacgctcttccgatctnnnnnnnnnnnnnnnnnnnnnnnnnnnnttttttttttttttttttttttttttttttnv

SEQ ID NO: 12 - DNA, 72 bp, unidentified
  source 1..72: mol_type = other DNA
gtcagatgtgtataagagacagnnnnnnnnnnnnnnnnnnnnnnnnnnnnttgctaggaccggccttaaagc

SEQ ID NO: 13 - DNA, 33 bp, unidentified
  source 1..33: mol_type = other DNA
gatcggaagagcacacgtctgaactccagtcac

SEQ ID NO: 14 - DNA, 13 bp, unidentified
  source 1..13: mol_type = other DNA
gctcttccgatct

SEQ ID NO: 15 - DNA, 88 bp, unidentified
  source 1..88: mol_type = other DNA
ctacacgacgctcttccgatctttttttttttttttttttttttttttttttvnagatcggaagagcacacgtctgaactccagtcac

SEQ ID NO: 16 - DNA, 67 bp, unidentified
  source 1..67: mol_type = other DNA
gctcttccgatctnvaaaaaaaaaaaaaaaaaaaaaaaaaaaaaaagatcggaagagcgtcgtgtag

SEQ ID NO: 17 - DNA, 44 bp, unidentified
  source 1..44: mol_type = other DNA
caagcagaagacggcatacgagagtgactggagttcagacgtgt

SEQ ID NO: 18 - DNA, 53 bp, unidentified
  source 1..53: mol_type = other DNA
aatgatacggcgaccaccgagatctacacacactctttccctacacgacgctc

SEQ ID NO: 19 - DNA, 58 bp, unidentified
  source 1..58: mol_type = other DNA
aatgatacggcgaccaccgagatctacactctttccctacacgacgctcttccgatct

SEQ ID NO: 20 - DNA, 143 bp, unidentified
  source 1..143: mol_type = other DNA
gtcagatgtgtataagagacagttgctaggaccggccttaaagcagagcccttgcgacttctcgcccatgtgtccgagcvvnnnnnnnnnnnnnnagagcccttgcgacttctnnnnnnnnagatcggaagagcgtcgtgtag

SEQ ID NO: 21 - DNA, 57 bp, unidentified
  source 1..57: mol_type = other DNA
caagcagaagacggcatacgagattcgtcggcagcgtcagatgtgtataagagacag

SEQ ID NO: 22 - RNA, 17 nt, unidentified
  source 1..17: mol_type = other RNA
aaaaaaatttttttttt

SEQ ID NO: 23 - protein, 13 aa, unidentified
CAEWYNQGGKLIF

SEQ ID NO: 24 - protein, 11 aa, unidentified
CASRASNEQFF

SEQ ID NO: 25 - DNA, 108 bp, unidentified
  source 1..108: mol_type = other DNA
tttgccggtaatacgactcactatagggctacacgacgctcttccgatctnnnnnnnncttcagcgttcccgagannnnnnnnnnnnnnvvgctcggacacatgggcg

SEQ ID NO: 26 - DNA, 150 bp, unidentified
  source 1..150: mol_type = other DNA
tttgccggtaatacgactcactatagggctacacgacgctcttccgatctnnnnnnnncttcagcgttcccgagannnnnnnnnnnnnnvvgctcggacacatgggcggagctttgctaacggtcgaggctttaaggccggtcctagcaa

SEQ ID NO: 27 - DNA, 86 bp, unidentified
  source 1..86: mol_type = other DNA
tttgtgactggagttcagacgtgtgctcttccgatcttcttcagcgttcccgagannnnnnnvvgctttaaggccggtcctagcaa

SEQ ID NO: 28 - DNA, 94 bp, unidentified
  source 1..94: mol_type = other DNA
tttgtgactggagttcagacgtgtgctcttccgatcttcttcagcgttcccgagannnnnnnvvaaaaaaaaaaaaaaaaaaaaaaaaaaaaaa